(19) United States
(12) Patent Application Publication
(10) Pub. No.: US 2020/0308590 A1
(43) Pub. Date: Oct. 1, 2020

(54) COMPOSITIONS AND METHODS FOR TREATMENT OF CYSTIC FIBROSIS

(71) Applicant: Yale University, New Haven, CT (US)

(72) Inventors: Peter M. Glazer, Guilford, CT (US); W. Mark Saltzman, New Haven, CT (US); Marie Egan, Madison, CT (US); Nicole Ali McNeer, Westport, CT (US)

(21) Appl. No.: 15/998,613

(22) PCT Filed: Feb. 16, 2017
(86) PCT No.: PCT/US2017/018165

§ 371 Date: Aug. 16, 2018

Related U.S. Application Data
(60) Provisional application No. 62/295,814, filed on Feb. 16, 2016.

(51) Int. Cl. C12N 15/113

(52) U.S. Cl. CPC C12N 15/1138; C12N 2310/3181; C12N 2310/15

(57) ABSTRACT

Compositions and methods of genome engineering in vitro and in vivo are provided. In some embodiments, the compositions are triplex forming molecules that bind or hybridize to a target region sequence in the human cystic fibrosis transmembrane conductance regulator (CFTR) gene. Preferably the triplex forming molecules are peptide nucleic acids that include a Hoogsteen binding peptide nucleic acid (PNA) segment and a Watson-Crick binding PNA segment collectively totaling no more than 50 nucleobases in length, wherein the two segments can binid or hybridize to a target region in the CFTR gene having a polypurine sequences and induce strand invasion, displacement, and formation of a triple-stranded molecule among the two PNA segments and the target region's sequence. Methods of using the triplex forming molecules to treat cystic fibrosis are also provided.

### CROSS-REFERENCE TO RELATED APPLICATIONS

This application is a National Phase application under 35 U.S.C. 371 of PCT/US2017/018165, filed Feb. 16, 2017 entitled “COMPOSITIONS AND METHODS FOR TREATMENT OF CYSTIC FIBROSIS,” which claims the benefit of and priority to U.S. Ser. No. 62/295,814 filed Feb. 16, 2016 and which are incorporated by referenced in their entirety.

### STATEMENT REGARDING FEDERALLY SPONSORED RESEARCH

This invention was made with government support under HL082655, HL110372, AI112443, EB000487 and GM007205 awarded by National Institutes of Health. The government has certain rights in the invention.

### REFERENCE TO SEQUENCE LISTING

The Sequence Listing submitted as a text file named “YU_6878_371_ST25.txt,” created on Jun. 18, 2020, and having a size of 76,344 bytes is hereby incorporated by reference pursuant to 37 C.F.R. § 1.52(e)(5).

### FIELD OF THE INVENTION

The field of the invention is generally related to triplex forming molecules and compositions and methods of use thereof for ex vivo and in vivo gene editing.

### BACKGROUND OF THE INVENTION

Cystic fibrosis (CF) is an autosomal recessive, multi-system disease caused by defects in the cystic fibrosis transmembrane conductance regulator (CFTR), an ion channel that mediates chloride transport. Lack of CFTR function causes obstructive lung disease, intestinal obstruction syndromes, liver dysfunction, exocrine and endocrine pancreatic dysfunction, and infertility. Since the sequencing and cloning of the CFTR gene in 1989 (Riordan, et al., Science, 245:1066-1073 (1989); Kerem, et al., Science, 245:1073-1080 (1989); Rommens, et al., Science, 245:1059-1065 (1989)), numerous mutations resulting in CF have been identified (Kerem, et al., Science, 245:1073-1080 (1989); Goetzinger, et al., Clinics in Laboratory Medicine, 30:533-543 (2010)). The most common mutation in CF is a three base-pair deletion (F508del) on chromosome 7, which results in the loss of a phenylalanine residue, causing increased degradation of the CFTR protein before it can reach the cell surface.

Although CF is one of the most rigorously characterized genetic diseases, current treatment of patients with CF focuses on symptomatic management rather than correction of the genetic defect. Some studies have demonstrated increased F508del activity with agents such as curcumin (Egan, et al., Science, 304:600-602 (2004); Cartiera, et al., Molecular Pharmaceutics, 7:86-93 (2010)) or histone deacetylase inhibitors (Hutt, et al., Nature Chemical Biology, 6:25-33 (2010)); VX-770 increases the activity of the CFTR protein in patients who have the less common G551D mutation. Gene therapy has remained unsuccessful in CF, due to challenges including in vivo delivery to the lung and other organ systems. In recent years, there have been many advances in gene therapy for treatment of diseases involving the hematolymphoid system, where harvest and ex vivo manipulation of cells for autologous transplantation is possible: examples include the use of zinc finger nucleases targeting CCR5 to produce HIV-1 resistant cells (Holt, et al., Nat Biotechnology, 28:839-847 (2010)), correction of the ABCD1 gene by lentiviral vectors (Cartier, et al., Science, 326:818-823 (2009)), and correction of SCID using retroviral gene transfer (Aiuti, et al., N Engl J Med., 360:447-458 (2009)). In contrast, harvest and autologous transplant is not a readily available option in CF, due to the involvement of the lung and other internal organs.

As one approach, the UK Cystic Fibrosis Gene Therapy Consortium is testing liposomes to deliver plasmids containing cDNA encoding CFTR to the lung. Other clinical trials have used viral vectors for delivery of the CFTR gene with limited success (reviewed in (Griesenbach, et al., Advanced Drug Delivery Reviews, 61:128-139 (2009)), or CFTR expression plasmids that are compacted by polyethylene glycol-substituted lysine 30-mer peptides (Konstan, et al., Human Gene Therapy, 15:1255-1269 (2004)). Delivery of plasmid DNA for gene addition without targeted insertion does not correct the endogenous gene and is not subject to normal CFTR gene regulation, while virus-mediated integration of the CFTR cDNA could introduce the risk of non-specific integration into important genomic sites. New gene delivery vectors include a chimeric Ad5F35 vector that showed much higher efficiency than traditional Ad5 vectors (Granio, et al., Human Gene Therapy, 21:251-269 (2010)). Researchers have demonstrated that treatment with the microRNA miR-138 leads to improved synthesis of CFTR-F508del (Ramachandran, et al., Proc Natl Acad Sci USA., 109:13362-13367 (2012)), and have also shown that lentiviruses can be used for gene transfer to porcine airways (Sinn, et al., Molecular Therapy Nucleic Acids, 1:e56 (2012)). Other current gene and cell therapy strategies have been recently reviewed (Oakland, et al., Mol Ther., 20:1108-1115 (2012)).

Current approaches for site-specific gene editing include short fragment homologous recombination using DNA fragments containing the correct CFTR sequence that can recombine with F508del CFTR genomic DNA, resulting in gene correction (Goncz, et al., Hum Mol Genet., 7:1913-1919 (1998); Goncz, et al., Gene Ther., 8:961-965 (2001); Bruscia, et al., Gene Ther., 9:683-685 (2002)), including introduction of the F508del mutation into normal mouse lung (Goncz, et al., Gene Ther., 8:961-965 (2001)). Zinc finger nucleases (ZFNs (Beumer, et al., Genetics, 172:2391-2403 (2006)) have recently been used to insert a CFTR transgene at the CCR5 locus 21 and for modification of F508del at levels <1% in vitro (Lee Ciaran, et al., BioResearch Open Access, 1:99-108 (2012)). CRISPR/Cas-9 technology has been used to correct F508del in intestinal organoids from CF patients in culture (Schwank, et al., Cell Stem Cell., 13:653-658 (2013)), but with high off-target effects (one out of twenty-five surveyed genes in a single analyzed clone). In addition, the efficiency of gene modification was low: approximately 0.3% of treated organoids (3 to 6/1400) had the desired modification (Schwank, et al., Cell Stem Cell., 13:653-658 (2013)). In vivo delivery is an important challenge, which was not attempted in this prior work with CRISPR/Cas9 or ZFNs.

Accordingly, there remains a need to improved compositions and methods for treating cystic fibrosis.

It is therefore an object of the invention to provide compositions and methods for achieved an increased frequency of gene modification in vivo.

It is a further object of the invention to provide compositions and methods that improve one or more symptoms of cystic fibrosis in a subject in need thereof.

### SUMMARY OF THE INVENTION

Cystic fibrosis (CF) is a lethal genetic disorder most commonly caused by the F508del mutation in the cystic fibrosis transmembrane conductance regulator (CFTR) gene. It is not readily amenable to gene therapy because of its systemic nature and challenges including in vivo gene delivery and transient gene expression. The results presented in the Examples below show that triplex-forming PNA molecules and donor DNA in biodegradable polymer nanoparticles can achieve in vitro and in vivo gene correction of the F508del mutation at an order of magnitude higher than previously achieved. Modification was confirmed with sequencing and a functional chloride efflux assay. In vitro correction of chloride efflux occurs in up to 25% of human cells, while deep sequencing reveals negligible off-target effects in partially homologous sites. Intranasal application of nanoparticles in CF mice produces changes in nasal epithelium potential differences consistent with corrected CFTR, and gene correction also detected in lung tissue.

Accordingly, compositions and methods of genome engineering in vitro and in vivo with oligonucleotides are provided. In some embodiments, the compositions are triplex forming molecules that bind or hybridize to a target region sequence in the human cystic fibrosis transmembrane conductance regulator (CFTR) gene having the sequence TTTCCTCT (SEQ ID NO:70), TTTCCTCTATGGGTAAG (SEQ ID NO:71), AGAGGAAA (SEQ ID NO:72), CTTACCCATAGAGGAAA (SEQ ID NO:73), AGAAGAGG (SEQ ID NO:74), ATGCCAACTAGAAGAGG (SEQ ID NO:75), CCTCTTCT (SEQ ID NO:76) or CCTCTTCTAGTTGGCAT (SEQ ID NO:77), CTTTCCCTT (SEQ ID NO:78), CTTTCCCTTGTATCTTTT (SEQ ID NO:79), AAGGGAAAG (SEQ ID NO:80), or AAAAGATAC AAGGGAAAG (SEQ ID NO:81).

In some embodiments, the triplex forming oligonucleotide is substantially complementary to the target region sequence and can form a triple helix with double-stranded DNA at the target sequence based on the third strand binding code.

In preferred embodiments, the triplex forming composition includes a Hoogsteen binding peptide nucleic acid (PNA) segment and a Watson-Crick binding PNA segment collectively totaling no more than 50 nucleobases in length, wherein the two segments can bind or hybridize to a target region sequence including

(i)
(SEQ ID NO: 72)
5′-AGAGGAAA-3′,
(ii)
(SEQ ID NO: 73)
5′-CTTACCCATAGAGGAAA-3′
(iii)
(SEQ ID NO: 74)
5′-AGAAGAGG-3′,
(iv)
(SEQ ID NO: 75)
5′-ATGCCAACTAGAAGAGG-3′,
(v)
(SEQ ID NO: 80)
5′-AAGGGAAAG-3′,
or
(iv)
(SEQ ID NO: 81)
5′-AAAAGATACAAGGGAAAG-3′,

in a cell's genome to induce strand invasion, displacement, and formation of a triple-stranded molecule among the two PNA segments and the target region's sequence. The Hoogsteen binding segment can bind to the target duplex by Hoogsteen binding for a length of least five nucleobases, and the Watson-Crick binding segment binds to the target duplex by Watson-Crick binding for a length of least five nucleobases. In some embodiments, the Hoogsteen binding segment includes one or more chemically modified cytosines selected from the group consisting of pseudocytosine, pseudoisocytosine, and 5-methylcytosine. The Watson-Crick binding segment can include a tail sequence of up to fifteen nucleobases that binds to the target duplex by Watson-Crick binding outside of the triplex. In preferred embodiments, the two segments are linked by a linker. The linker can be, for example, between about 1 and 10 units of 8-amino-3,6-dioxaoctanoic acid.
For example, in some embodiments, the

(i) the Hoogsteen binding segment comprises the sequence TJTJJTTT (SEQ ID NO:91) and the Watson-Crick binding segment comprises the sequence TTTCCTCT (SEQ ID NO:83) or TTTCCTCTATGGGTAAG (SEQ ID NO:84);

(ii) the Hoogsteen binding segment comprises the sequence TJTTJTJJ (SEQ ID NO:177) and the Watson-Crick binding segment comprises the sequence CCTCTTCT (SEQ ID NO:86), or CCTCTTCTAGTTGGCAT (SEQ ID NO:87); or

(iii) the Hoogsteen binding segment comprises the sequence TTJJJTTTJ (SEQ ID NO:92) and the Watson-Crick binding segment comprises the sequence CTTTCCCTT (SEQ ID NO:89), or CTTTCCCTTGTATCTTTT (SEQ ID NO:90);

wherein “J” is pseudoisocytosine.

In more specific embodiments, the triplex forming PNA has the sequence

(i)(hCFPNA2)
(SEQ ID NO: 93)
lys-lys-lys-TJTJJTTT-OOO-TTTCCTCTATGGGTAAG-lys-
lys-lys;
(ii)(hCFPNA1)
(SEQ ID NO: 94)
lys-lys-lys-TJTTJTJJ-OOO-CCTCTTCTAGTTGGCAT-lys-
lys-lys;
or
(iii)(hCFPNA3)
(SEQ ID NO: 95)
lys-lys-lys-TTJJJTTTJ-OOO-CTTTCCCTTGTATCTTTT-
lys-lys-lys.

The triplex forming molecules, the donor oligonucleotide, or a combination thereof are packaged together or separately in nanoparticles. The nanoparticles can include poly(lactic-co-glycolic acid) (PLGA). The nanoparticles can be a blend of PLGA and PBAE, for example a blend having between about 10 and about 20 percent PBAE (wt %). The nanoparticle can be prepared by double emulsion.

In some embodiments, a targeting moiety, a cell penetrating peptide, or a combination thereof associated with, linked, conjugated, or otherwise attached directly or indirectly to the triplex forming molecules, the donor oligonucleotides, the nanoparticles or a combination thereof. In a particular embodiments, the cell penetrating peptide includes the sequence GALFLGFLGAAGSTMGAWS QPKKKRKV (SEQ ID NO:12) (MPG (Synthetic chimera: SV40 Lg T. Ant.+HIV gb41 coat)). In some embodiments the compositions are target to the nasal or lung epithelium. In some embodiments, the lung progenitor cells are targeted.

Methods of use are also provided. For example, a method of modifying the human cystic fibrosis transmembrane conductance regulator (CFTR) gene in a cell can include administering a subject with a mutation in the CFTR gene an effective amount of the triplex forming composition to increase correction of the mutation in a population of cells relative to contacting the cells with donor oligonucleotide alone. In some embodiments, the composition is administered by intranasal or pulmonary delivery. The composition can induce or enhance gene correction in an effective amount to reduce one or more symptoms of cystic fibrosis. For example, the treatment can improve impaired response to cyclic AMP stimulation, improve hyperpolarization in response to forskolin, reduction in the large lumen negative nasal potential, reduce inflammatory cells in the bronchioalveolar lavage (BAL), improve lung histology, or a combination thereof.

### BRIEF DESCRIPTION OF THE DRAWINGS

FIG. 1A is an illustration of the overall strategy for PNA-induced recombination and gene correction, and detection of modification by AS-PCR. FIG. 1B is a schematic of hCFPNA1 ((ii) lys-lys-lys-TJTTJTJJ-OOO-CCTCTTCTAGTTGGCAT-lys-lys-lys (SEQ ID NO:94) (hCFPNA1)) forming a PNA/DNA/PNA triplex the human CFTR gene (5′ CCTCTTCTAGTTGGCAT 3′ (SEQ ID NO:77) and (5′ ATGCCAACTAGAAGAGG 3′ (SEQ ID NO:75)). hCFPNA1 binds 54 bp downstream of the F508DEL target site. FIG. 1C is a schematic of hCFPNA2 (lys-lys-lys-TJTJJTTT-OOO-TTTCCTCTATGGGTAAG-lys-lys-lys (SEQ ID NO:93) (hCFPNA2)) forming a PNA/DNA/PNA triplex with the human CFTR gene ((5′ TTTCCTCTATGGGTAAG 3′ (SEQ ID NO:71) and 5′ CTTACCCATAGAGGAAA 3′ (SEQ ID NO:73)). hCFPNA2 binds 178 bp downstream of the F508DEL target site. FIG. 1D is a schematic of hCFPNA3 (lys-lys-lys-TTJJJTTTJ-OOO-CTTTCCCTTGTATCTTTT-lys-lys-lys (SEQ ID NO:95) (hCFPNA3)) forming a PNA/DNA/PNA triplex with the human CFTR gene ((5′ CTTTCCCTTGTATCTTTT 3′ (SEQ ID NO:79) and 5′ AAAAGATACAAGGGAAAG 3′ (SEQ ID NO:81)). hCFPNA3 binds 317 bp upstream of the F508DEL target site. FIG. 1E is a schematic of mCFPNA2 ((ls-lys-lys-JTTTTJJJ-OOO-CCCTTTTCAAGGTGAGTAG-lys-lys-lys) (SEQ ID NO:69)) forming a PNA/DNA/PNA triplex with the mouse CFTR gene ((5′CCCTTTTCAAGGTGAGTAG 3′ (SEQ ID NO:67) and 5′ CTACTCACCTTGAAAAGGG 3′ (SEQ ID NO:68)). For FIGS. 1B-1E, “J” represents pseudoisocytosine, a C analog for improved triplex formation at physiologic pH.

FIG. 2A is an illustration showing an assay for isolation of corrected cells by limiting dilution and cloning into multi-well plates. Cells were plated at dilutions ranging from 100 cells/well to 1 cell/well. After expansion to produce enough cells for harvest, genomic DNA was extracted from each well, and AS-PCR used to detect presence of the corrected CFTR sequence. FIG. 2B is a line graphs showing chloride efflux measured using N-[ethoxycarbonylmethyl]-6-methoxy-quinolinium bromide (MQAE), a fluorescent indicator dye over time (seconds). Example traces from untreated CFBE41o− cells (n=23) (bottom) and a corrected CFBE clone (n=26) (top) are shown. Error bars=standard error of the mean. FIG. 2C is a bar graph showing a summary of chloride efflux: cell-averaged arbitrary fluorescence units per minute (AFU/min) for untreated CFBE cells (n=138), blank treated cells (n=168), modified clones (n=108 for clone 105, n=100 for clone 411), and wild type 16HBE14o− cells (n=113). Error bars=standard error of the mean.

FIG. 3A is a line graph showing cumulative release (OD/mg/ml) of nucleic acid from PLGA nanoparticles with DNA alone or PNA:DNA loading ratio of 1:2 at 37° C. FIG. 3B is a line graph showing cumulative release (OD/mg/ml) of nucleic acid from PLGA/PBAE/MPG particles with hCFPNA2 (SEQ ID NO:93):DNA (SEQ ID NO:96) loading ratio of 2:1 at 37° C. Average sizes of particles were analyzed by ImageJ of SEM images: diameters were 120+/−40 nm for blank, 150+/−55 nm for CFDNA, 120+/−27 for CFPNA1, 140+/−72 for hCFPNA2, and 130+/−42 for hCFPNA3 particles.

FIG. 4A is a bar graph summarizing chloride efflux: cell-averaged arbitrary fluorescence units per minute (AFU/min) for untreated CFBE cells (n=138), treated cells (n=150), and wildtype 16HBE14o− cells (n=113). CFPNA2 NPs=cell population treated with PLGA nanoparticles containing hCFPNA2 (SEQ ID NO:93) and donor DNA (SEQ ID NO:96). CFPNA2 Modified NPs=cell population treated with PLGA/PBAE/MPG nanoparticles containing hCFPNA2 (SEQ ID NO:93) and donor DNA (SEQ ID NO:96). p=0.003 two-tailed Fisher's exact test between PLGA and PBAE/PLGA/MPG treated cells. Error bars show the SD. FIG. 4B is a pair of line graphs showing the change in NPD (mV) in mice treated by intranasal infusion with nanoparticles. Nasal potential difference measurements were assessed prior to nanoparticle treatment, and subsequent to treatment. The response to a 0Cl+amiloride+forksolin perfusate after nanoparticle treatment was compared to the response prior to treatment. Each data point represents one mouse, with a line connecting pre and post-treatment values. Mice treated with PLGA (left panel) or PLGA/PBAE/MPG nanoparticles (right panel) containing PNA/DNA are shown. Pre and post treatment changes in NPD were compared using paired t tests for each mouse. FIG. 4C is a series of dot plots showing nasal potential difference changes (mV) in functional and control nanoparticle treated CF mice. Each mouse is represented with an individual data point; in addition, the mean is shown with a horizontal line, surrounded by error bars showing the standard error of the mean. Pre and post treatment changes in NPD were compared using unpaired t tests for each group. In the last panel, nasal potential difference changes in wild type mice are shown for comparison. FIG. 4D is a plot showing chloride efflux measured using N-[ethoxycarbonylmethyl]-6-methoxy-quinolinium bromide (MQAE), a fluorescent indicator dye (Intensity of Fluorescence (AFU)). Cells (n=24) were treated as in FIG. 4A, but with PLGA/PBAE/MPG nanoparticles containing PNA/DNA targeting the human β-globin gene or with PNA targeting CFTR and DNA targeting β-globin. Error bars=standard error of the mean. FIGS. 4E and 4F are bar graphs showing baseline NPD (4E) and amiloride response (4F) in CF, treated-CF, and wildtype mice prior to and subsequence to treatment by intranasal infusion with nanoparticles. Wild-type mice (n=6), untreated CF mice (n=18), CF mice treated with PLGA (CF+PNA) (n=8) or PLGA/PBAE/MPG nanoparticles (CF+PNA-MPG) (n=8) containing PNA/DNA are shown. All error bars show SD; measurements were compared between groups using one way ANOVA with multiple comparisons.

FIG. 5 is a bar graph showing cytokine production in bronchioalveolar lavage fluid of treated and control mice, with BAL from LPS treated control mice shown as a positive control, using LUMINEX® bead-based assay.

FIG. 6A is a bar graph showing the results of deep sequencing in additional human genomic sites in cells treated 3 times with 2 mg/mL PLGA/PBAE/MPG PNA/DNA nanoparticle compared to untreated controls. The total number of aligned sequences were queried and at each of the 13 off-target sites, the percentage of sequences that had 0 to 5 mismatched base pairs was calculated with average and standard deviation. FIG. 6B is a box-whisker plot showing the results of a Comet assay for DNA damage. CFBE cells treated for 24 hours with 2 mg/mL DNA-containing PLGA/PBAE/MPG nanoparticles, 2 mg/mL PNA and DNA-containing PLGA/PBAE/MPG nanoparticles, or 2 ug of hCas9 plasmid (Addgene plasmid 41815), prepared per the TREVIGEN® COMETASSAY® protocol and comet tail moments were calculated using TriTek CometScore FreeWare. Plots show the median comet tail moments (horizontal lines), min and max comet tail moments (top and bottom of vertical lines), and first to third quartile (box). P-values are for Student's test, two-tailed, unpaired, unequal variance.

### DETAILED DESCRIPTION OF THE INVENTION

### I. Definitions

As used herein, “affinity tags” are defined herein as molecular species which form highly specific, non-covalent, physiochemical interactions with defined binding partners. Affinity tags which form highly specific, non-covalent, physiochemical interactions with one another are defined herein as “complementary”.

As used herein, “coupling agents” are defined herein as molecular entities which associate with polymeric nanoparticles and provide substrates that facilitate the modular assembly and disassembly of functional elements onto the nanoparticle. Coupling agents can be conjugated to affinity tags. Affinity tags allow for flexible assembly and disassembly of functional elements which are conjugated to affinity tags that form highly specific, noncovalent, physiochemical interactions with affinity tags conjugated to adaptor elements. Coupling agents can also be covalently coupled to functional elements in the absence of affinity tags.

As used herein, the term “isolated” describes a compound of interest (e.g., either a polynucleotide or a polypeptide) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein with respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “host cell” refers to prokaryotic and eukaryotic cells into which a nucleic acid can be introduced.

As used herein, “transformed” and “transfected” encompass the introduction of a nucleic acid into a cell by one of a number of techniques known in the art.

As used herein, the phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Specific binding between two entities means an affinity of at least 10⁶, 10⁷, 10⁸, 10⁹, or 10¹⁰ M⁻¹. Affinities greater than 10⁸ M⁻¹ are preferred.

As used herein, “targeting molecule” is a substance which can direct a nanoparticle to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

As used herein, the terms “antibody” or “immunoglobulin” are used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes a bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin. Exp. Immunol., 79:315-321 (1990); Kostelny, et al., J. Immunol., 148, 1547-1553 (1992).

As used herein, the terms “epitope” or “antigenic determinant” refer to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10, amino acids, in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke, et al., J. Inf Dis., 170:1110-19 (1994)), by antigen-dependent killing (cytotoxic T lymphocyte assay, Tigges, et al., J. Immunol., 156, 3901-3910) or by cytokine secretion.

As used herein, the term “small molecule,” as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

### II. Gene Editing Potentiating Factors

It has been discovered that certain potentiating factors can be used to increase the efficacy of gene editing technologies. Gene expression profiling on SCF-treated CD117+ cells versus untreated CD117+ cells discussed in the Examples below showed additional up-regulation of numerous DNA repair genes including RAD51 and BRCA2. These results and others discussed below indicate that a functional c-Kit signaling pathway mediates increased HDR and promotes gene editing, rather than CD117 simply being a phenotypic marker. When CD117+ cells were treated with SCF, expression of these DNA repair genes was increased even more, correlating with a further increase in gene editing.

Accordingly, compositions and methods of increasing the efficacy of gene editing technology are provided. As used herein a “gene editing potentiating factor” or “gene editing potentiating agent” or “potentiating factor or “potentiating agent” refers a compound that increases the efficacy of editing (e.g., mutation, including insertion, deletion, substitution, etc.) of a gene, genome, or other nucleic acid) by a gene editing technology relative to use of the gene editing technology in the absence of the compound. Preferred gene editing technologies suitable for use alone or more preferably in combination with the disclosed potentiating factors are discussed in more detail below. In certain preferred embodiments, the gene editing technology is a triplex-forming yPNA and donor DNA, optionally, but preferably in a nanoparticle composition.

Potentiating factors include, for example, DNA damage or repair-stimulating or -potentiating factors. Preferably the factor is one that engages one or more endogenous high fidelity DNA repair pathways. In some embodiments, the factor is one that increases expression of Rad51, BRCA2, or a combination thereof.

As discussed in more detail below, the preferred methods typically include contacting cells with an effective amount of a gene editing potentiating factor. The contacting can occur ex vivo, for example isolated cells, or in vivo following, for example, administration of the potentiating factor to a subject.

A. C-Kit Ligands

In some embodiments, the factor is an activator of the receptor tyrosine kinase c-Kit. CD117 (also known as mast/stem cell growth factor receptor or proto-oncogene c-Kit protein) is a receptor tyrosine kinase expressed on the surface of hematopoietic stem and progenitor cells as well as other cell types. Stem cell factor (SCF), the ligand for c-Kit, causes dimerization of the receptor and activates its tyrosine kinase activity to trigger downstream signaling pathways that can impact survival, proliferation, and differentiation. SCF and c-Kit are reviewed in Lennartsson and Rönnstrand, Physiological Reviews, 92(4):1619-1649 (2012)).

The human SCF gene encodes for a 273 amino acid transmembrane protein, which contains a 25 amino acid N-terminal signal sequence, a 189 amino acid extracellular domain, a 23 amino acid transmembrane domain, and a 36 amino acid cytoplasmic domain. A canonical human SCF amino acid sequence is:

(SEQ ID NO: 1, UniProtKB-P21583 (SCF_HUMAN))
MKKTQTWILTCIYLQLLLFNPLVKTEGICRNRVTNNVKDVTKLVANLP
KDYMITLKYVPGMDVLPSHCWISEMVVQLSDSLTDLLDKFSNISEGLS
NYSIIDKLVNIVDDLVECVKENSSKDLKKSFKSPEPRLFTPEEFFRIF
NRSIDAFKDFVVASETSDCVVSSTLSPEKDSRVSVTKPFMLPPVAASS
LRNDSSSSNRKAKNPPGDSSLHWAAMALPALFSLIIGFAFGALYWKKR
QPSLTRAVENIQINEEDNEISMLQEKEREFQEV.

The secreted soluble form of SCF is generated by proteolytic processing of the membrane-anchored precursor. A cleaved, secreted soluble form of human SCF is underlined in SEQ ID NO: 1, which corresponds to SEQ ID NO:2 without the N-terminal methionine. MEGICRNRVTNNVKDVTKLVANLPKDYMITLKYVPGMDVLPSHCWI SEMVVQLSDSLTDLLDKFSNISEGLSNYSIIDKLVNIVDDLVECVKEN SSKDLKKSFKSPEPRLFTPEEFFRIFNRSIDAFKDFVVASETSDCVVSST LSPEKD SRVSVTKPFMLPPVA (SEQ ID NO:2, Preprotech Recombinant Human SCF Catalog Number: 300-07).

Murine and rat SCF are fully active on human cells. A canonical mouse SCF amino acid sequence is:

(SEQ ID NO: 3, UniProtKB-P20826 (SCF_MOUSE))
MKKTQTWIITCIYLQLLLFNPLVKTKEICGNPVTDNVKDITKLVANLP
NDYMITLNYVAGMDVLPSHCWLRDMVIQLSLSLTTLLDKFSNISEGLS
NYSIIDKLGKIVDDLVLCMEENAPKNIKESPKRPETRSFTPEEFFSIF
NRSIDAFKDFMVASDTSDCVLSSTLGPEKDSRVSVTKPFMLPPVAASS
LRNDSSSSNRKAAKAPEDSGLQWTAMALPALISLVIGFAFGALYWKKK
QSSLTRAVENIQINEEDNEISMLQQKEREFQEV.

A cleaved, secreted soluble form of mouse SCF is underlined in SEQ ID NO:3, which corresponds to SEQ ID NO:4 without the N-terminal methionine.

### MKEICGNPVTDNVKDITKLVANLPNDYMITLNYVAGMDVLPSHCWL RDMVIQLSLSLTTLLDKFSNISEGLSNYSIIDKLGKIVDDLVLCMEENA PKNIKESPKRPETRSFTPEEFFSIFNRSIDAFKDFMVASDTSDCVLSSTL GPEKDSRVSVTKPFMLPPVA (SEQ ID NO:4, Preprotech Recombinant Murine SCF Catalog Number: 250-03)

A canonical mouse SCF amino acid sequence is:

(SEQ ID NO: 5, UniProtKB-P21581 (SCF_RAT))
MKKTQTWIITCIYLQLLLFNPLVKTQEICRNPVTDNVKDITKLVANLP
NDYMITLNYVAGMDVLPSHCWLRDMVTHLSVSLTTLLDKFSNISEGLS
NYSIIDKLGKIVDDLVACMEENAPKNVKESLKKPETRNFTPEEFFSIF
NRSIDAFKDFMVASDTSDCVLSSTLGPEKDSRVSVTKPFMLPPVAASS
LRNDSSSSNRKAAKSPEDPGLQWTAMALPALISLVIGFAFGALYWKKK
QSSLTRAVENIQINEEDNEISMLQQKEREFQEV.

A cleaved, secreted soluble form of rat SCF is underlined in SEQ ID NO:5, which corresponds to SEQ ID NO:6 without the N-terminal methionine.

MQEICRNPVTDNVKDITKLVANLPNDYMITLNYVAGMDVLPSHCWL RDMVTHLSVSLTTLLDKFSNISEGLSNYSIIDKLGKIVDDLVACMEEN APKNVKESLKKPETRNFTPEEFFSIFNRSIDAFKDFMVASDTSDCVLSS TLGPEKDSRVSVTKPFMLPPVA (SEQ ID NO:6, Shenandoah Biotechnology, Inc., Recombinant Rat SCF (Stem Cell Factor) Catalog Number: 300-32).

In some embodiments, the factor is a SCF such as any of SEQ ID NO: 1-6, with or without the N-terminal methionine, or a functional fragment thereof, or a variant thereof with at least 60, 65, 70, 75, 80, 85, 90, 95, 96, 97, 98, 99, or more sequence identity to any one of SEQ ID NO:1-6.

It will be appreciated that SCF can be administered to cells or a subject as SCF protein, or as a nucleic acid encoding SCF (transcribed RNA, DNA, DNA in an expression vector). Accordingly, nucleic acid sequences, including RNA (e.g., mRNA) and DNA sequences, encoding SEQ ID NOS:1-6 are also provided, both alone and inserted into expression cassettes and vectors. For example, a sequence encoding SCF can be incorporated into an autonomously replicating plasmid, a virus (e.g., a retrovirus, lentivirus, adenovirus, or herpes virus), or into the genomic DNA of a prokaryote or eukaryote.

The observed effect of SCF indicates that other cytokines or growth factors including, but not limited to, erythropoietin, GM-CSF, EGF (especially for epithelial cells; lung epithelia for cystic fibrosis), hepatocyte growth factor etc., could similarly serve to boost gene editing potential in bone marrow cells or in other tissues. In some embodiments, gene editing is enhanced in specific cell types using cytokines targeted to these cell types.

B. Replication Modulators

In some embodiments, the potentiating factor is a replication modulator that can, for example, manipulate replication progression and/or replication forks. For example, the ATR-Chkl cell cycle checkpoint pathway has numerous roles in protecting cells from DNA damage and stalled replication, one of the most prominent being control of the cell cycle and prevention of premature entry into mitosis (Thompson and Eastman, Br J Clin Pharmacol., 76(3): 358-369 (2013), Smith, et al., Adv Cancer Res., 108:73-112 (2010)). However, Chkl also contributes to the stabilization of stalled replication forks, the control of replication origin firing and replication fork progression, and homologous recombination. DNA polymerase alpha also known as Pol a is an enzyme complex found in eukaryotes that is involved in initiation of DNA replication. Hsp90 (heat shock protein 90) is a chaperone protein that assists other proteins to fold properly, stabilizes proteins against heat stress, and aids in protein degradation.

Experimental results show that inhibitors of CHK1 and ATR in the DNA damage response pathway, as well as DNA polymerase alpha inhibitors and HSP90 inhibitors, substantially boost gene editing by triplex-forming PNAs and single-stranded donor DNA oligonucleotides. Accordingly, in some embodiments, the potentiating factor is a CHK1 or ATR pathway inhibitor, a DNA polymerase alpha inhibitor, or an HSP90 inhibitor. The inhibitor can be a functional nucleic acid, for example siRNA, miRNA, aptamers, ribozymes, triplex forming molecules, RNAi, or external guide sequences that targets CHK1, ATR, or another molecule in the ATR-Chkl cell cycle checkpoint pathway; DNA polymerase alpha; or HSP90 and reduces expression or active of ATR, CHK1, DNA polymerase alpha, or HSP90.

Preferably, the inhibitor is a small molecule. For example, the potentiating factor can be a small molecule inhibitor of ATR-Chkl Cell Cycle Checkpoint Pathway Inhibitor. Such inhibitors are known in the art, and many have been tested in clinical trials for the treatment of cancer. Exemplary CHK1 inhibitors include, but are not limited to, AZD7762, SCH900776/MK-8776, IC83/LY2603618, LY2606368, GDC-0425, PF-00477736, XL844, CEP-3891, SAR-020106, CCT-244747, Arry-575 (Thompson and Eastman, Br J Clin Pharmacol., 76(3): 358-369 (2013)), and SB218075. Exemplary ATR pathway inhibitors include, but are not limited to Schisandrin B, NU6027, NVP-BEZ235, VE-821, VE-822 (VX-970), AZ20, AZD6738, MIRIN, KU5593, VE-821, NU7441, LCA, and L189 (Weber and Ryan, Pharmacology & Therapeutics, 149:124-138 (2015)).

In some embodiments, the potentiating factor is a DNA polymerase alpha inhibitor, such as aphidicolin.

In some embodiments, the potentiating factor is a heat shock protein 90 inhibitor (HSP90i) such as STA-9090 (ganetespib). Other HSP90 inhibitors are known in the art and include, but are not limited to, benzoquinone ansamycin antibiotics such as geldanamycin (GA); 17-AAG (17-Allylamino-17-demethoxy-geldanamycin); 17-DMAG (17-dimethylaminoethylamino-17-demethoxy-geldanamycin) (Alvespimycin); IPI-504 (Retaspimycin); and AUY922 (Tatokoro, et al., EXCLI J., 14:48-58 (2015)).

### III. Gene Editing Technology

Gene editing technologies can be used alone or preferably in combination with a potentiating agent. Exemplary gene editing technologies include, but are not limited to, triplex-forming, pseudocomplementary oligonucleotides, CRISPR/Cas, zinc finger nucleases, and TALENs, each of which are discussed in more detail below. As discussed in more detail below, some gene editing technologies are used in combination with a donor oligonucleotide. In some embodiments, the gene editing technology is the donor oligonucleotide, which can be used be used alone to modify genes. Strategies include, but are not limited to, small fragment homologous replacement (e.g., polynucleotide small DNA fragments (SDFs)), single-stranded oligodeoxynucleotide-mediated gene modification (e.g., ssODN/SSOs) and other described in Sargent, Oligonucleotides, 21(2): 55-75 (2011)), and elsewhere. Other suitable gene editing technologies include, but are not limited to intron encoded meganucleases that are engineered to change their target specificity. See, e.g., Arnould, et al., Protein Eng. Des. Sel., 24(1-2):27-31 (2011)).

A. Triplex-Forming Molecules

1. Compositions

Compositions containing “triplex-forming molecules,” that bind to duplex DNA in a sequence-specific manner to form a triple-stranded structure include, but are not limited to, triplex-forming oligonucleotides (TFOs), peptide nucleic acids (PNA), and “tail clamp” PNA (tcPNA). The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids. Triplex forming molecules are described in U.S. Pat. Nos. 5,962,426, 6,303,376, 7,078,389, 7,279,463, 8,658,608, U.S. Published Application Nos. 2003/0148352, 2010/0172882, 2011/0268810, 2011/0262406, 2011/0293585, and published PCT application numbers WO 1995/001364, WO 1996/040898, WO 1996/039195, WO 2003/052071, WO 2008/086529, WO 2010/123983, WO 2011/053989, WO 2011/133802, WO 2011/13380, Rogers, et al., Proc Natl Acad Sci USA, 99:16695-16700 (2002), Majumdar, et al., Nature Genetics, 20:212-214 (1998), Chin, et al., Proc Natl Acad Sci USA, 105:13514-13519 (2008), and Schleifman, et al., Chem Biol., 18:1189-1198 (2011). As discussed in more detail below, triplex forming molecules are typically single-stranded oligonucleotides that bind to polypyrimidine:polypurine target motif in a double stranded nucleic acid molecule to form a triple-stranded nucleic acid molecule. The single-stranded oligonucleotide typically includes a sequence substantially complementary to the polypurine strand of the polypyrimidine:polypurine target motif.

a. Triplex-Forming Oligonucleotides (TFOs)

Triplex-forming oligonucleotides (TFOs) are defined as oligonucleotides which bind as third strands to duplex DNA in a sequence specific manner. The oligonucleotides are synthetic or isolated nucleic acid molecules which selectively bind to or hybridize with a predetermined target sequence, target region, or target site within or adjacent to a human gene so as to form a triple-stranded structure.

Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful.

The oligonucleotides are preferably generated using known DNA synthesis procedures. In one embodiment, oligonucleotides are generated synthetically. Oligonucleotides can also be chemically modified using standard methods that are well known in the art.

The nucleotide sequence of the oligonucleotides is selected based on the sequence of the target sequence, the physical constraints imposed by the need to achieve binding of the oligonucleotide within the major groove of the target region, and the need to have a low dissociation constant (K_{d}) for the oligonucleotide/target sequence. The oligonucleotides have a base composition which is conducive to triple-helix formation and is generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C+.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

Preferably, the oligonucleotide binds to or hybridizes to the target sequence under conditions of high stringency and specificity. Most preferably, the oligonucleotides bind in a sequence-specific manner within the major groove of duplex DNA. Reaction conditions for in vitro triple helix formation of an oligonucleotide probe or primer to a nucleic acid sequence vary from oligonucleotide to oligonucleotide, depending on factors such as oligonucleotide length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, a triplex forming molecule is said to be substantially complementary to a target region when the oligonucleotide has a heterocyclic base composition which allows for the formation of a triple-helix with the target region. As such, an oligonucleotide is substantially complementary to a target region even when there are non-complementary bases present in the oligonucleotide. As stated above, there are a variety of structural motifs available which can be used to determine the nucleotide sequence of a substantially complementary oligonucleotide.

b. Peptide Nucleic Acids (PNA)

In another embodiment, the triplex-forming molecules are peptide nucleic acids (PNAs). Peptide nucleic acids are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that are similar to oligonucleotides, but are achiral and neutrally charged molecules. Peptide nucleic acids are comprised of peptide nucleic acid monomers. The heterocyclic bases can be any of the standard bases (uracil, thymine, cytosine, adenine and guanine) or any of the modified heterocyclic bases described below.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules, or two PNA molecules linked together by a linker of sufficient flexibility to form a single bis-PNA molecule. In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to, 8-amino-3,6-dioxaoctanoic acid, referred to as an O-linker, and 6-aminohexanoic acid. Poly(ethylene) glycol monomers can also be used in bis-PNA linkers. A bis-PNA linker can contain multiple linker molecule monomers in any combination.

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand.

c. Tail Clamp Peptide Nucleic Acids (tcPNA)

Although polypurine:polypyrimidine stretches do exist in mammalian genomes, it is desirable to target triplex formation in the absence of this requirement. In some embodiments such as PNA, triplex-forming molecules include a “tail” added to the end of the Watson-Crick binding portion. Adding additional nucleobases, known as a “tail” or “tail clamp”, to the Watson-Crick binding portion that bind to the target strand outside the triple helix further reduces the requirement for a polypurine:polypyrimidine stretch and increases the number of potential target sites. The tail is most typically added to the end of the Watson-Crick binding sequence furthest from the linker. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. U.S.A., 99(26):16695-700 (2002)).

Tails added to clamp PNAs (sometimes referred to as bis-PNAs) form tail-clamp PNAs (referred to as tcPNAs) that have been described by Kaihatsu, et al., Biochemistry, 42(47):13996-4003 (2003); Bentin, et al., Biochemistry, 42(47):13987-95 (2003). tcPNAs are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site compared to PNA without the tail.

d. PNA Modifications

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand. Common modifications to PNA are discussed in Sugiyama and Kittaka, Molecules, 18:287-310 (2013)) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to DNA to generate a chimeric oligomer, redesign of the backbone architecture, conjugation of PNA to DNA or RNA. These modifications improve solubility but often result in reduced binding affinity and/or sequence specificity.

In some embodiments, the some or all of the PNA monomers are modified at the gamma position in the polyamide backbone (yPNAs) as illustrated below (wherein “B” is a nucleobase and “R” is a substitution at the gamma position).

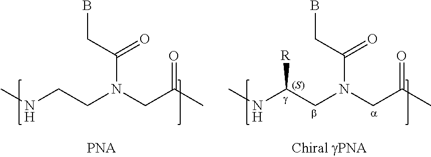

Substitution at the gamma position creates chirality and provides helical pre-organization to the PNA oligomer, yielding substantially increased binding affinity to the target DNA (Rapireddy, et al., Biochemistry, 50(19):3913-8 (2011)). Other advantageous properties can be conferred depending on the chemical nature of the specific substitution at the gamma position (the “R” group in the chiral γPNA above).

One class of γ substitution is miniPEG, but other residues and side chains can be considered, and even mixed substitutions can be used to tune the properties of the oligomers. “MiniPEG” and “MP” refers to diethylene glycol. MiniPEG-containing γPNAs are conformationally preorganized PNAs that exhibit superior hybridization properties and water solubility as compared to the original PNA design and other chiral γPNAs. γPNAs prepared from L-amino acids adopt a right-handed helix, while those prepared from D-amino acids adopt a left-handed helix; however, only the right-handed helical γPNAs hybridize to DNA or RNA with high affinity and sequence selectivity. In the most preferred embodiments, some or all of the PNA monomers are miniPEG-containing γPNAs (Sahu, et al., J. Org. Chem., 76, 5614-5627 (2011). In the embodiments, tcPNAs are prepared wherein every other PNA monomer on the Watson-Crick binding side of the linker is a miniPEG-containing γPNA. Accordingly, the tail clamp side of the PNA has alternating PNA and miniPEG-containing γPNA monomers.

In some embodiments PNA-mediated gene editing are achieved via additional or alternative γ substitutions or other PNA chemical modifications including but limited to those introduced above and below. Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

In addition to γPNAs showing consistently improved gene editing potency the level of off-target effects in the genome remains extremely low. This is in keeping with the lack of any intrinsic nuclease activity in the PNAs (in contrast to ZFNs or CRISPR/Cas9 or TALENS), and reflects the mechanism of triplex-induced gene editing, which acts by creating an altered helix at the target-binding site that engages endogenous high fidelity DNA repair pathways. As discussed above, the SCF/c-Kit pathway also stimulates these same pathways, providing for enhanced gene editing without increasing off-target risk or cellular toxicity.

Additionally, any of the triplex forming sequences can be modified to include guanidine-G-clamp (“G-clamp”) PNA monomer(s) to enhance PNA binding. γPNAs with substitution of cytosine by clamp-G (9-(2-guanidinoethoxy) phenoxazine), a cytosine analog that can form five H-bonds with guanine, and can also provide extra base stacking due to the expanded phenoxazine ring system and substantially increased binding affinity. In vitro studies indicate that a single clamp-G substitution for C can substantially enhance the binding of a PNA-DNA duplex by 23° C. (Kuhn, et al., Artificial DNA, PNA & XNA, 1(1):45-53(2010)). As a result, γPNAs containing G-clamp substitutions can have further increased activity.

The structure of a clamp-G monomer-to-G base pair (clamp-G indicated by the “X”) is illustrated below in comparison to C-G base pair.

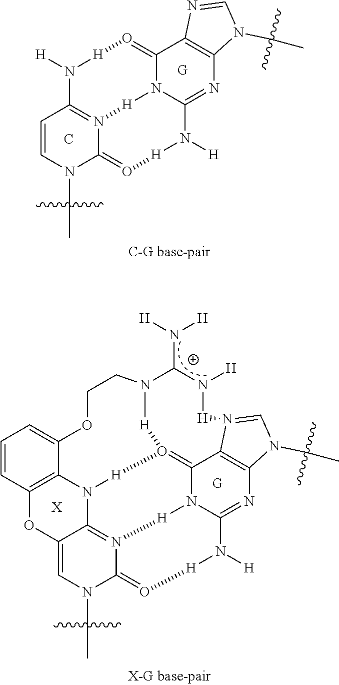

Some studies have shown improvements using D-amino acids in peptide synthesis.

2. Triplex-Forming Target Sequence Considerations

The triplex-forming molecules bind to a predetermined target region referred to herein as the “target sequence,” “target region,” or “target site.” The target sequence for the triplex-forming molecules can be within or adjacent to a human gene encoding, for example the beta globin, cystic fibrosis transmembrane conductance regulator (CFTR) or other gene discussed in more detail below, or an enzyme necessary for the metabolism of lipids, glycoproteins, or mucopolysaccharides, or another gene in need of correction. The target sequence can be within the coding DNA sequence of the gene or within an intron. The target sequence can also be within DNA sequences which regulate expression of the target gene, including promoter or enhancer sequences or sites that regulate RNA splicing.

The nucleotide sequences of the triplex-forming molecules are selected based on the sequence of the target sequence, the physical constraints, and the need to have a low dissociation constant (K_{d}) for the triplex-forming molecules/target sequence. As used herein, triplex-forming molecules are said to be substantially complementary to a target region when the triplex-forming molecules has a heterocyclic base composition which allows for the formation of a triple-helix with the target region. As such, a triplex-forming molecules is substantially complementary to a target region even when there are non-complementary bases present in the triplex-forming molecules.

There are a variety of structural motifs available which can be used to determine the nucleotide sequence of a substantially complementary oligonucleotide. Preferably, the triplex-forming molecules bind to or hybridize to the target sequence under conditions of high stringency and specificity. Reaction conditions for in vitro triple helix formation of an triplex-forming molecules probe or primer to a nucleic acid sequence vary from triplex-forming molecules to triplex-forming molecules, depending on factors such as the length triplex-forming molecules, the number of G:C and A:T base pairs, and the composition of the buffer utilized in the hybridization reaction.

a. Target Sequence Considerations for TFOs

Preferably, the TFO is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful. Most preferably, the oligonucleotides bind in a sequence-specific manner within the major groove of duplex DNA. An oligonucleotide substantially complementary, based on the third strand binding code, to the target region of the double-stranded nucleic acid molecule is preferred. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C+.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

The oligonucleotides are preferably generated using known DNA synthesis procedures. In one embodiment, oligonucleotides are generated synthetically. Oligonucleotides can also be chemically modified using standard methods that are well known in the art.

b. Target Sequence Considerations for PNAs

Some triplex-forming molecules, such as PNA and tcPNA invade the target duplex, with displacement of the polypyrimidine strand, and induce triplex formation with the polypurine strand of the target duplex by both Watson-Crick and Hoogsteen binding. Preferably, both the Watson-Crick and Hoogsteen binding portions of the triplex forming molecules are substantially complementary to the target sequence. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Preferably, PNAs are between 6 and 50 nucleotides in length. The Watson-Crick portion should be 9 or more nucleobases in length, optionally including a tail sequence. More preferably, the Watson-Crick binding portion is between about 9 and 30 nucleobases in length, optionally including a tail sequence of between 0 and about 15 nucleobases. More preferably, the Watson-Crick binding portion is between about 10 and 25 nucleobases in length, optionally including a tail sequence of between 0 and about 10 nucleobases. In the most preferred embodiment, the Watson-Crick binding portion is between 15 and 25 nucleobases in length, optionally including a tail sequence of between 5 and 10 nucleobases. The Hoogsteen binding portion should be 6 or more nucleobases in length. Most preferably, the Hoogsteen binding portion is between about 6 and 15 nucleobases, inclusive.

The triplex-forming molecules are designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobases, known as a “tail,” to the Watson-Crick binding portion of the triplex-forming molecules allows the Watson-Crick binding portion to bind/hybridize to the target strand outside the site of polypurine sequence for triplex formation. These additional bases further reduce the requirement for the polypurine:polypyrimidine stretch in the target duplex and therefore increase the number of potential target sites. Triplex-forming oligonucleotides (TFOs) also require a polypurine:polypyrimidine sequence to a form a triple helix. TFOs may require stretch of at least 15 and preferably 30 or more nucleotides. Peptide nucleic acids require fewer purines to a form a triple helix, although at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

The triple-forming molecules are preferably generated using known synthesis procedures. In one embodiment, triplex-forming molecules are generated synthetically. Triplex-forming molecules can also be chemically modified using standard methods that are well known in the art.

B. Pseudocomplementary Oligonucleotides

The gene editing technology can be pseudocomplementary oligonucleotides such as those disclosed in U.S. Pat. No. 8,309,356. “Double duplex-forming molecules,” are oligonucleotides that bind to duplex DNA in a sequence-specific manner to form a four-stranded structure. Double duplex-forming molecules, such as a pair of pseudocomplementary oligonucleotides, can induce recombination with a donor oligonucleotide at a chromosomal site in mammalian cells. Pseudocomplementary oligonucleotides are complementary oligonucleotides that contain one or more modifications such that they do not recognize or hybridize to each other, for example due to steric hindrance, but each can recognize and hybridize to its complementary nucleic acid strands at the target site. Preferred pseudocomplementary oligonucleotides include Pseudocomplementary peptide nucleic acids (pcPNAs). A pseudocomplementary oligonucleotide is said to be substantially complementary to a target region when the oligonucleotide has a base composition which allows for the formation of a double duplex with the target region. As such, an oligonucleotide is substantially complementary to a target region even when there are non-complementary bases present in the oligonucleotide.

This strategy can be more efficient and provides increased flexibility over other methods of induced recombination such as triple-helix oligonucleotides and bis-peptide nucleic acids which prefer a polypurine sequence in the target double-stranded DNA. The design ensures that the pseudocomplementary oligonucleotides do not pair with each other but instead bind the cognate nucleic acids at the target site, inducing the formation of a double duplex.

The predetermined region that the double duplex-forming molecules bind to can be referred to as a “double duplex target sequence,” “double duplex target region,” or “double duplex target site.” The double duplex target sequence (DDTS) for the double duplex-forming oligonucleotides can be, for example, within or adjacent to a human gene in need of induced gene correction. The DDTS can be within the coding DNA sequence of the gene or within introns. The DDTS can also be within DNA sequences which regulate expression of the target gene, including promoter or enhancer sequences.

The nucleotide sequence of the pseudocomplementary oligonucleotides is selected based on the sequence of the DDTS. Therapeutic administration of pseudocomplementary oligonucleotides involves two single stranded oligonucleotides unlinked, or linked by a linker. One pseudocomplementary oligonucleotide strand is complementary to the DDTS, while the other is complementary to the displaced DNA strand. The use of pseudocomplementary oligonucleotides, particularly pcPNAs are not subject to limitation on sequence choice and/or target length and specificity as are triplex-forming oligonucleotides, helix-invading peptide nucleic acids (bis-PNAs) and side-by-side minor groove binders. Pseudocomplementary oligonucleotides do not require third-strand Hoogsteen-binding, and therefore are not restricted to homopurine targets. Pseudocomplementary oligonucleotides can be designed for mixed, general sequence recognition of a desired target site. Preferably, the target site contains an A:T base pair content of about 40% or greater. Preferably pseudocomplementary oligonucleotides are between about 8 and 50 nucleobases, more preferably 8 to 30, even more preferably between about 8 and 20 nucleobases.

The pseudocomplementary oligonucleotides should be designed to bind to the target site (DDTS) at a distance of between about 1 to 800 bases from the target site of the donor oligonucleotide. More preferably, the pseudocomplementary oligonucleotides bind at a distance of between about 25 and 75 bases from the donor oligonucleotide. Most preferably, the pseudocomplementary oligonucleotides bind at a distance of about 50 bases from the donor oligonucleotide. Preferred pcPNA sequences for targeted repair of a mutation in the β-globin intron IVS2 (G to A) are described in U.S. Pat. No. 8,309,356.

Preferably, the pseudocomplementary oligonucleotides bind/hybridize to the target nucleic acid molecule under conditions of high stringency and specificity. Most preferably, the oligonucleotides bind in a sequence-specific manner and induce the formation of double duplex. Specificity and binding affinity of the pseudocomplemetary oligonucleotides may vary from oligonucleotide to oligonucleotide, depending on factors such as oligonucleotide length, the number of G:C and A:T base pairs, and the formulation.

C. CRISPR/Cas

In some embodiments, the gene editing composition is the CRISPR/Cas system. CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the “target sequence” and the tracrRNA is often referred to as the “scaffold.”

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence (such as CTPS1) can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

D. Zinc Finger Nucleases

In some embodiments, the element that induces a single or a double strand break in the target cell's genome is a nucleic acid construct or constructs encoding a zinc finger nucleases (ZFNs). ZFNs are typically fusion proteins that include a DNA-binding domain derived from a zinc-finger protein linked to a cleavage domain.

The most common cleavage domain is the Type IIS enzyme Fok1. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys₂His₂ zinc fingers, each of which generally recognizes three to four nucleotides in the target DNA sequence. The Cys₂His₂ domain has a general structure: Phe (sometimes Tyr)-Cys-(2 to 4 amino acids)-Cys-(3 amino acids)-Phe(sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

E. Transcription Activator-Like Effector Nucleases

In some embodiments, the element that induces a single or a double strand break in the target cell's genome is a nucleic acid construct or constructs encoding a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). U.S. Published Application No. 2011/0145940, which discloses TAL effectors and methods of using them to modify DNA. Miller et al. Nature Biotechnol 29: 143 (2011) reported making TALENs for site-specific nuclease architecture by linking TAL truncation variants to the catalytic domain of Fok1 nuclease. The resulting TALENs were shown to induce gene modification in immortalized human cells. General design principles for TALE binding domains can be found in, for example, WO 2011/072246.

### IV. Donor Oligonucleotides

In some embodiments, the gene editing composition includes or is administered in combination with a donor oligonucleotide. Generally, in the case of gene therapy, the donor oligonucleotide includes a sequence that can correct a mutation(s) in the host genome, though in some embodiments, the donor introduces a mutation that can, for example, reduce expression of an oncogene or a receptor that facilitates HIV infection. In addition to containing a sequence designed to introduce the desired correction or mutation, the donor oligonucleotide may also contain synonymous (silent) mutations (e.g., 7 to 10). The additional silent mutations can facilitate detection of the corrected target sequence using allele-specific PCR of genomic DNA isolated from treated cells.

A. Preferred Donor Oligonucleotide Design for Triplex and Double-Duplex Based Technologies

The triplex forming molecules including peptide nucleic acids may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence is between 1 to 800 bases from the target binding site of the triplex-forming molecules. More preferably the donor oligonucleotide sequence is between 25 to 75 bases from the target binding site of the triplex-forming molecules. Most preferably that the donor oligonucleotide sequence is about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the oligonucleotide donor is between 25 and 80 nucleobases. In a further embodiment, the non-tethered donor nucleotide is about 50 to 60 nucleotides in length.

The donor oligonucleotides contain at least one mutated, inserted or deleted nucleotide relative to the target DNA sequence. Target sequences can be within the coding DNA sequence of the gene or within introns. Target sequences can also be within DNA sequences which regulate expression of the target gene, including promoter or enhancer sequences or sequences that regulate RNA splicing.

The donor oligonucleotides can contain a variety of mutations relative to the target sequence. Representative types of mutations include, but are not limited to, point mutations, deletions and insertions. Deletions and insertions can result in frameshift mutations or deletions. Point mutations can cause missense or nonsense mutations. These mutations may disrupt, reduce, stop, increase, improve, or otherwise alter the expression of the target gene.

Compositions including triplex-forming molecules such as tcPNA may include one or more than one donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic bases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. Modifications to the donor oligonucleotide should not prevent the donor oligonucleotide from successfully recombining at the recombination target sequence in the presence of triplex-forming molecules.

B. Preferred Donor Oligonucleotides Design for Nuclease-Based Technologies

The nuclease activity of the genome editing systems described herein cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Therefore, in some embodiments, the genome editing composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence by cleaving the target DNA sequence and allowing the cell to repair the sequence in the absence of an exogenously provided donor polynucleotide. Alternatively, if the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy.

In applications in which it is desirable to insert a polynucleotide sequence into a target DNA sequence, a polynucleotide including a donor sequence to be inserted is also provided to the cell. By a “donor sequence” or “donor polynucleotide” or “donor oligonucleotide” it is meant a nucleic acid sequence to be inserted at the cleavage site. The donor polynucleotide typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence flanked by two regions of homology, such that homology-directed repair between the target DNA region and the two flanking sequences results in insertion of the non-homologous sequence at the target region.

### V. Oligonucleotide Composition

Any of the gene editing technologies, components thereof, donor oligonucleotides, or other nucleic acids disclosed herein can include one or more modifications or substitutions to the nucleobases or linkages. Although modifications are particularly preferred for use with triplex-forming technologies and typically discussed below with reference thereto, any of the modifications can be utilized in the construction of any of the disclosed gene editing compositions, donor, nucleotides, etc. Modifications should not prevent, and preferably enhance the activity, persistence, or function of the gene editing technology. For example, modifications to oligonucleotides for use as triplex-forming should not prevent, and preferably enhance duplex invasion, strand displacement, and/or stabilize triplex formation as described above by increasing specificity or binding affinity of the triplex-forming molecules to the target site. Modified bases and base analogues, modified sugars and sugar analogues and/or various suitable linkages known in the art are also suitable for use in the molecules disclosed herein. Several preferred oligonucleotide compositions including PNA, and modification thereof to include MiniPEG at the γ position in the PNA backbone, are discussed above. Additional modifications are discussed in more detail below.

A. Heterocyclic Bases

The principal naturally-occurring nucleotides include uracil, thymine, cytosine, adenine and guanine as the heterocyclic bases. Gene editing molecules can include chemical modifications to their nucleotide constituents. For example, target sequences with adjacent cytosines can be problematic. Triplex stability is greatly compromised by runs of cytosines, thought to be due to repulsion between the positive charge resulting from the N³ protonation or perhaps because of competition for protons by the adjacent cytosines. Chemical modification of nucleotides including triplex-forming molecules such as PNAs may be useful to increase binding affinity of triplex-forming molecules and/or triplex stability under physiologic conditions.

Chemical modifications of heterocyclic bases or heterocyclic base analogs may be effective to increase the binding affinity of a nucleotide or its stability in a triplex. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in triplex-forming molecules such as PNAs helps to stabilize triplex formation at neutral and/or physiological pH, especially in triplex-forming molecules with isolated cytosines. This is because the positive charge partially reduces the negative charge repulsion between the triplex-forming molecules and the target duplex, and allows for Hoogsteen binding.

B. Backbone

The nucleotide subunits of the triplex-forming molecules such as PNAs are connected by an internucleotide bond that refers to a chemical linkage between two nucleoside moieties. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds, which allow them to form PNA-DNA or PNA-RNA duplexes via Watson-Crick base pairing with high affinity and sequence-specificity. PNAs maintain spacing of heterocyclic bases that is similar to conventional DNA oligonucleotides, but are achiral and neutrally charged molecules. Peptide nucleic acids are composed of peptide nucleic acid monomers.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Backbone modifications used to generate triplex-forming molecules should not prevent the molecules from binding with high specificity to the target site and creating a triplex with the target duplex nucleic acid by displacing one strand of the target duplex and forming a clamp around the other strand of the target duplex.

C. Modified Nucleic Acids

Modified nucleic acids in addition to peptide nucleic acids are also useful as triplex-forming molecules. Oligonucleotides are composed a chain of nucleotides which are linked to one another. Canonical nucleotides typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds. As used herein “modified nucleotide” or “chemically modified nucleotide” defines a nucleotide that has a chemical modification of one or more of the heterocyclic base, sugar moiety or phosphate moiety constituents. Preferably the charge of the modified nucleotide is reduced compared to DNA or RNA oligonucleotides of the same nucleobase sequence. Most preferably the triplex-forming molecules have low negative charge, no charge, or positive charge such that electrostatic repulsion with the nucleotide duplex at the target site is reduced compared to DNA or RNA oligonucleotides with the corresponding nucleobase sequence.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

Molecules may also include nucleotides with modified heterocyclic bases, sugar moieties or sugar moiety analogs. Modified nucleotides may include modified heterocyclic bases or base analogs as described above with respect to peptide nucleic acids. Sugar moiety modifications include, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O—(N-(methyl)acetamido) (2′-OMA). 2′-O-aminoethyl sugar moiety substitutions are especially preferred because they are protonated at neutral pH and thus suppress the charge repulsion between the triplex-forming molecule and the target duplex. This modification stabilizes the C3′-endo conformation of the ribose or deoxyribose and also forms a bridge with the i−1 phosphate in the purine strand of the duplex.

### VI. Nanoparticle Delivery Vehicles

Any of the disclosed compositions including, but not limited to potentiating factors, gene editing molecules, donor oligonucleotides, etc., can be delivered to the target cells using a nanoparticle delivery vehicle. In some embodiments, some of the compositions are packaged in nanoparticles and some are not. For example, in some embodiments, the gene editing technology and/or donor oligonucleotide is incorporated into nanoparticles while the potentiating factor is not. In some embodiments, the gene editing technology and/or donor oligonucleotide, and the potentiating factor are packaged in nanoparticles. The different compositions can be packaged in the same nanoparticles or different nanoparticles. For example, the compositions can be mixed and packaged together. In some embodiments, the different compositions are packaged separately into separate nanoparticles wherein the nanoparticles are similarly or identically composed and/or manufactured. In some embodiments, the different compositions are packaged separately into separate nanoparticles wherein the nanoparticles are differentially composed and/or manufactured.

Nanoparticles generally refers to particles in the range of between 500 nm to less than 0.5 nm, preferably having a diameter that is between 50 and 500 nm, more preferably having a diameter that is between 50 and 300 nm. Cellular internalization of polymeric particles is highly dependent upon their size, with nanoparticulate polymeric particles being internalized by cells with much higher efficiency than microparticulate polymeric particles. For example, Desai, et al. have demonstrated that about 2.5 times more nanoparticles that are 100 nm in diameter are taken up by cultured Caco-2 cells as compared to microparticles having a diameter on 1 μM (Desai, et al., Pharm. Res., 14:1568-73 (1997)). Nanoparticles also have a greater ability to diffuse deeper into tissues in vivo.

A. Polymer

The polymer that forms the core of the nanoparticle may be any biodegradable or non-biodegradable synthetic or natural polymer. In a preferred embodiment, the polymer is a biodegradable polymer. Nanoparticles are ideal materials for the fabrication of gene editing delivery vehicles: 1) control over the size range of fabrication, down to 100 nm or less, an important feature for passing through biological barriers; 2) reproducible biodegradability without the addition of enzymes or cofactors; 3) capability for sustained release of encapsulated, protected nucleic acids over a period in the range of days to months by varying factors such as the monomer ratios or polymer size, for example, the ratio of lactide to glycolide monomer units in poly(lactide-co-glycolide) (PLGA); 4) well-understood fabrication methodologies that offer flexibility over the range of parameters that can be used for fabrication, including choices of the polymer material, solvent, stabilizer, and scale of production; and 5) control over surface properties facilitating the introduction of modular functionalities into the surface.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), and poly(amine-co-ester) polymers, such as those described in Zhou, et al., Nature Materials, 11:82-90 (2012) and WO 2013/082529, U.S. Published Application No. 2014/0342003, and PCT/US2015/061375.

Preferred natural polymers include alginate and other polysaccharides, collagen, albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

In some embodiments, non-biodegradable polymers can be used, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers include, but are not limited to, polyanhydrides, polyamides, polycarbonates, polyalkylenes, polyalkylene oxides such as polyethylene glycol, polyalkylene terepthalates such as poly(ethylene terephthalate), polyvinyl alcohols, polyvinyl ethers, polyvinyl esters, polyethylene, polypropylene, poly(vinyl acetate), poly vinyl chloride, polystyrene, polyvinyl halides, polyvinylpyrrolidone, polymers of acrylic and methacrylic esters, polysiloxanes, polyurethanes and copolymers thereof, modified celluloses, alkyl cellulose, hydroxyalkyl celluloses, cellulose ethers, cellulose esters, nitro celluloses, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate). These materials may be used alone, as physical mixtures (blends), or as copolymers.

The polymer may be a bioadhesive polymer that is hydrophilic or hydrophobic. Hydrophilic polymers include CARBOPOL™ (a high molecular weight, crosslinked, acrylic acid-based polymers manufactured by NOVEON™), polycarbophil, cellulose esters, and dextran.

Release rate controlling polymers may be included in the polymer matrix or in the coating on the formulation. Examples of rate controlling polymers that may be used are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

These polymers can be obtained from sources such as Sigma Chemical Co., St. Louis, Mo.; Polysciences, Warrenton, Pa.; Aldrich, Milwaukee, Wis.; Fluka, Ronkonkoma, N.Y.; and BioRad, Richmond, Calif., or can be synthesized from monomers obtained from these or other suppliers using standard techniques.

In a preferred embodiment, the nanoparticles are formed of polymers fabricated from polylactides (PLA) and copolymers of lactide and glycolide (PLGA). These have established commercial use in humans and have a long safety record (Jiang, et al., Adv. Drug Deliv. Rev., 57(3):391-410); Aguado and Lambert, Immunobiology, 184(2-3):113-25 (1992); Bramwell, et al., Adv. Drug Deliv. Rev., 57(9):1247-65 (2005)). These polymers have been used to encapsulate siRNA (Yuan, et al., Jour. Nanosocience and Nanotechnology, 6:2821-8 (2006); Braden, et al., Jour. Biomed. Nanotechnology, 3:148-59 (2007); Khan, et al., Jour. Drug Target, 12:393-404 (2004); Woodrow, et al., Nature Materials, 8:526-533 (2009)). Murata, et al., J. Control. Release, 126(3):246-54 (2008) showed inhibition of tumor growth after intratumoral injection of PLGA microspheres encapsulating siRNA targeted against vascular endothelial growth factor (VEGF). However, these microspheres were too large to be endocytosed (35-45 μm) (Conner and Schmid, Nature, 422(6927):37-44 (2003)) and required release of the anti-VEGF siRNA extracellularly as a polyplex with either polyarginine or PEI before they could be internalized by the cell. These microparticles may have limited applications because of the toxicity of the polycations and the size of the particles. Nanoparticles (100-300 nm) of PLGA can penetrate deep into tissue and are easily internalized by many cells (Conner and Schmid, Nature, 422(6927):37-44 (2003)).

The nanoparticles can be designed to release encapsulated nucleic acids over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity, affecting degradation rate. Specifically, the hydrophobic poly (lactic acid) (PLA), more hydrophilic poly (glycolic acid) PGA and their copolymers, poly (lactide-co-glycolide) (PLGA) have various release rates. The degradation rate of these polymers, and often the corresponding drug release rate, can vary from days (PGA) to months (PLA) and is easily manipulated by varying the ratio of PLA to PGA.

Exemplary nanoparticles are described in U.S. Pat. Nos. 4,883,666, 5,114,719, 5,601,835, 7,534,448, 7,534,449, 7,550,154, and 8,889,117, and U.S. Published Application Nos. 2009/0269397, 2009/0239789, 2010/0151436, 2011/0008451, 2011/0268810, 2014/0342003, 2015/0118311, 2015/0125384, 2015/0073041, Hubbell, et al., Science, 337:303-305 (2012), Cheng, et al., Biomaterials, 32:6194-6203 (2011), Rodriguez, et al., Science, 339:971-975 (2013), Hrkach, et al., Sci Transl Med., 4:128ra139 (2012), McNeer, et al., Mol Ther., 19:172-180 (2011), McNeer, et al., Gene Ther., 20:658-659 (2013), Babar, et al., Proc Natl Acad Sci USA, 109:E1695-E1704 (2012), Fields, et al., J Control Release 164:41-48 (2012), and Fields, et al., Advanced Healthcare Materials, 361-366 (2015).

B. Polycations

In a preferred embodiment, the nucleic acids are complexed to polycations to increase the encapsulation efficiency of the nucleic acids into the nanoparticles. The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values.

Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quartemized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In one embodiment, the polycation is a polyamine. Polyamines are compounds having two or more primary amine groups. In a preferred embodiment, the polyamine is a naturally occurring polyamine that is produced in prokaryotic or eukaryotic cells. Naturally occurring polyamines represent compounds with cations that are found at regularly-spaced intervals and are therefore particularly suitable for complexing with nucleic acids. Polyamines play a major role in very basic genetic processes such as DNA synthesis and gene expression. Polyamines are integral to cell migration, proliferation and differentiation in plants and animals. The metabolic levels of polyamines and amino acid precursors are critical and hence biosynthesis and degradation are tightly regulated. Suitable naturally occurring polyamines include, but are not limited to, spermine, spermidine, cadaverine and putrescine. In a preferred embodiment, the polyamine is spermidine.

In another embodiment, the polycation is a cyclic polyamine. Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane) which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

In some embodiments, the particles themselves are a polycation (e.g., a blend of PLGA and poly(beta amino ester).

C. Coupling Agents or Ligands

The external surface of the polymeric nanoparticles may be modified by conjugating to, or incorporating into, the surface of the nanoparticle a coupling agent or ligand.

In a preferred embodiment, the coupling agent is present in high density on the surface of the nanoparticle. As used herein, “high density” refers to polymeric nanoparticles having a high density of ligands or coupling agents, which is preferably in the range of 1,000 to 10,000,000, more preferably 10,000-1,000,000 ligands per square micron of nanoparticle surface area. This can be measured by fluorescence staining of dissolved particles and calibrating this fluorescence to a known amount of free fluorescent molecules in solution.

Coupling agents associate with the polymeric nanoparticles and provide substrates that facilitate the modular assembly and disassembly of functional elements to the nanoparticles. Coupling agents or ligands may associate with nanoparticles through a variety of interactions including, but not limited to, hydrophobic interactions, electrostatic interactions and covalent coupling.

In a preferred embodiment, the coupling agents are molecules that match the polymer phase hydrophile-lipophile balance. Hydrophile-lipophile balances range from 1 to 15. Molecules with a low hydrophile-lipophile balance are more lipid loving and thus tend to make a water in oil emulsion while those with a high hydrophile-lipophile balance are more hydrophilic and tend to make an oil in water emulsion. Fatty acids and lipids have a low hydrophile-lipophile balance below 10.

Any amphiphilic polymer with a hydrophile-lipophile balance in the range 1-10, more preferably between 1 and 6, most preferably between 1 and up to 5, can be used as a coupling agent. Examples of coupling agents which may associate with polymeric nanoparticles via hydrophobic interactions include, but are not limited to, fatty acids, hydrophobic or amphipathic peptides or proteins, and polymers. These classes of coupling agents may also be used in any combination or ratio. In a preferred embodiment, the association of adaptor elements with nanoparticles facilitates a prolonged presentation of functional elements which can last for several weeks.

Coupling agents can also be attached to polymeric nanoparticles through covalent interactions through various functional groups. Functionality refers to conjugation of a molecule to the surface of the particle via a functional chemical group (carboxylic acids, aldehydes, amines, sulfhydryls and hydroxyls) present on the surface of the particle and present on the molecule to be attached.

Functionality may be introduced into the particles in two ways. The first is during the preparation of the nanoparticles, for example during the emulsion preparation of nanoparticles by incorporation of stabilizers with functional chemical groups. Suitable stabilizers include hydrophobic or amphipathic molecules that associate with the outer surface of the nanoparticles.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a molecule such as a protein. The reaction is an N-nucleophilic substitution and results in a stable N-alkylcarbamate linkage of the molecule to the polymer. The “coupling” of the molecule to the “activated” polymer matrix is maximal in the pH range of 9-10 and normally requires at least 24 hrs. The resulting molecule-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of molecules in a totally aqueous environment at the physiological pH of 7.0. Briefly, EDAC and sulfo-NHS form an activated ester with the carboxylic acid groups of the polymer which react with the amine end of a molecule to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the molecule-polymer complex.

By using either of these protocols it is possible to “activate” almost all polymers containing either hydroxyl or carboxyl groups in a suitable solvent system that will not dissolve the polymer matrix.

A useful coupling procedure for attaching molecules with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of molecules and polymers with double bonds, including the use of UV crosslinking, may be used for attachment of molecules to the polymer.

In one embodiment, coupling agents can be conjugated to affinity tags. Affinity tags are any molecular species which form highly specific, noncovalent, physiochemical interactions with defined binding partners. Affinity tags which form highly specific, noncovalent, physiochemical interactions with one another are defined herein as “complementary”. Suitable affinity tag pairs are well known in the art and include epitope/antibody, biotin/avidin, biotin/streptavidin, biotin/neutravidin, glutathione-S-transferase/glutathione, maltose binding protein/amylase and maltose binding protein/maltose. Examples of suitable epitopes which may be used for epitope/antibody binding pairs include, but are not limited to, HA, FLAG, c-Myc, glutatione-S-transferase, His₆, GFP, DIG, biotin and avidin. Antibodies (both monoclonal and polyclonal and antigen-binding fragments thereof) which bind to these epitopes are well known in the art.

Affinity tags that are conjugated to coupling agents allow for highly flexible, modular assembly and disassembly of functional elements which are conjugated to affinity tags which form highly specific, noncovalent, physiochemical interactions with complementary affinity tags which are conjugated to coupling agents. Adaptor elements may be conjugated with a single species of affinity tag or with any combination of affinity tag species in any ratio. The ability to vary the number of species of affinity tags and their ratios conjugated to adaptor elements allows for exquisite control over the number of functional elements which may be attached to the nanoparticles and their ratios.

In another embodiment, coupling agents are coupled directly to functional elements in the absence of affinity tags, such as through direct covalent interactions. Coupling agents can be covalently coupled to at least one species of functional element. Coupling agents can be covalently coupled to a single species of functional element or with any combination of species of functional elements in any ratio.

In a preferred embodiment, coupling agents are conjugated to at least one affinity tag that provides for assembly and disassembly of modular functional elements which are conjugated to complementary affinity tags. In a more preferred embodiment, coupling agents are fatty acids that are conjugated with at least one affinity tag. In a particularly preferred embodiment, the coupling agents are fatty acids conjugated with avidin or streptavidin. Avidin/streptavidin-conjugated fatty acids allow for the attachment of a wide variety of biotin-conjugated functional elements.

The coupling agents are preferably provided on, or in the surface of, nanoparticles at a high density. This high density of coupling agents allows for coupling of the polymeric nanoparticles to a variety of species of functional elements while still allowing for the functional elements to be present in high enough numbers to be efficacious.

1. Fatty Acids

The coupling agents may include fatty acids. Fatty acids may be of any acyl chain length and may be saturated or unsaturated. In a particularly preferred embodiment, the fatty acid is palmitic acid. Other suitable fatty acids include, but are not limited to, saturated fatty acids such as butyric, caproic, caprylic, capric, lauric, myristic, stearic, arachidic and behenic acid. Still other suitable fatty acids include, but are not limited to, unsaturated fatty acids such as oleic, linoleic, alpha-linolenic, arachidonic, eicosapentaenoic, docosahexaenoic and erucic acid.

2. Hydrophobic or Amphipathic Peptides

The coupling agents may include hydrophobic or amphipathic peptides. Preferred peptides should be sufficiently hydrophobic to preferentially associate with the polymeric nanoparticle over the aqueous environment. Amphipathic polypeptides useful as adaptor elements may be mostly hydrophobic on one end and mostly hydrophilic on the other end. Such amphipathic peptides may associate with polymeric nanoparticles through the hydrophobic end of the peptide and be conjugated on the hydrophilic end to a functional group.

3. Hydrophobic Polymers

Coupling agents may include hydrophobic polymers. Examples of hydrophobic polymers include, but are not limited to, polyanhydrides, poly(ortho)esters, and polyesters such as polycaprolactone.

### VII. Functional Molecules

Functional molecules can be associated with, linked, conjugated, or otherwise attached directly or indirectly gene editing technology, potentiating agents, or nanoparticles utilized for delivery thereof.

A. Targeting Molecules

One class of functional elements is targeting molecules. Targeting molecules can be associated with, linked, conjugated, or otherwise attached directly or indirectly to the gene editing molecule, or to a nanoparticle or other delivery vehicle thereof.

Targeting molecules can be proteins, peptides, nucleic acid molecules, saccharides or polysaccharides that bind to a receptor or other molecule on the surface of a targeted cell. The degree of specificity and the avidity of binding to the graft can be modulated through the selection of the targeting molecule. For example, antibodies are very specific. These can be polyclonal, monoclonal, fragments, recombinant, or single chain, many of which are commercially available or readily obtained using standard techniques.

Examples of moieties include, for example, targeting moieties which provide for the delivery of molecules to specific cells, e.g., antibodies to hematopoietic stem cells, CD34⁺ cells, T cells or any other preferred cell type, as well as receptor and ligands expressed on the preferred cell type. Preferably, the moieties target hematopoeitic stem cells.

Examples of molecules targeting extracellular matrix (“ECM”) include glycosaminoglycan (“GAG”) and collagen. In one embodiment, the external surface of polymer particles may be modified to enhance the ability of the particles to interact with selected cells or tissue. The method described above wherein an adaptor element conjugated to a targeting molecule is inserted into the particle is preferred. However, in another embodiment, the outer surface of a polymer micro- or nanoparticle having a carboxy terminus may be linked to targeting molecules that have a free amine terminus.

Other useful ligands attached to polymeric micro- and nanoparticles include pathogen-associated molecular patterns (PAMPs). PAMPs target Toll-like Receptors (TLRs) on the surface of the cells or tissue, or signal the cells or tissue internally, thereby potentially increasing uptake. PAMPs conjugated to the particle surface or co-encapsulated may include: unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

In another embodiment, the outer surface of the particle may be treated using a mannose amine, thereby mannosylating the outer surface of the particle. This treatment may cause the particle to bind to the target cell or tissue at a mannose receptor on the antigen presenting cell surface. Alternatively, surface conjugation with an immunoglobulin molecule containing an Fc portion (targeting Fc receptor), heat shock protein moiety (HSP receptor), phosphatidylserine (scavenger receptors), and lipopolysaccharide (LPS) are additional receptor targets on cells or tissue.

Lectins that can be covalently attached to micro- and nanoparticles to render them target specific to the mucin and mucosal cell layer include lectins isolated from Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The choice of targeting molecule will depend on the method of administration of the nanoparticle composition and the cells or tissues to be targeted. The targeting molecule may generally increase the binding affinity of the particles for cell or tissues or may target the nanoparticle to a particular tissue in an organ or a particular cell type in a tissue. Avidin increases the ability of polymeric nanoparticles to bind to tissues. While the exact mechanism of the enhanced binding of avidin-coated particles to tissues has not been elucidated, it is hypothesized it is caused by electrostatic attraction of positively charged avidin to the negatively charged extracellular matrix of tissue. Non-specific binding of avidin, due to electrostatic interactions, has been previously documented and zeta potential measurements of avidin-coated PLGA particles revealed a positively charged surface as compared to uncoated PLGA particles.

The attachment of any positively charged ligand, such as polyethyleneimine or polylysine, to any polymeric particle may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most particles with the appropriate chemistry and be expected to influence the binding of particles to the gut. For example, polyclonal antibodies raised against components of mucin or else intact mucin, when covalently coupled to particles, would provide for increased bioadhesion. Similarly, antibodies directed against specific cell surface receptors exposed on the lumenal surface of the intestinal tract would increase the residence time of beads, when coupled to particles using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the particles would decrease the surface tension of the bead-gut interface and increase the solubility of the bead in the mucin layer. The list of useful ligands includes, but is not limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range yields chains of 120 to 425 amino acid residues attached to the surface of the particles. The polyamino chains increase bioadhesion by means of chain entanglement in mucin strands as well as by increased carboxylic charge.

The efficacy of the nanoparticles is determined in part by their route of administration into the body. For orally and topically administered nanoparticles, epithelial cells constitute the principal barrier that separates an organism's interior from the outside world. Epithelial cells such as those that line the gastrointestinal tract form continuous monolayers that simultaneously confront the extracellular fluid compartment and the extracorporeal space.

Adherence to cells is an essential first step in crossing the epithelial barrier by any of these mechanisms. Therefore, in one embodiment, the nanoparticles disclosed herein further include epithelial cell targeting molecules. Epithelial cell targeting molecules include monoclonal or polyclonal antibodies or bioactive fragments thereof that recognize and bind to epitopes displayed on the surface of epithelial cells. Epithelial cell targeting molecules also include ligands which bind to a cell surface receptor on epithelial cells. Ligands include, but are not limited to, molecules such as polypeptides, nucleotides and polysaccharides.

A variety of receptors on epithelial cells may be targeted by epithelial cell targeting molecules. Examples of suitable receptors to be targeted include, but are not limited to, IgE Fc receptors, EpCAM, selected carbohydrate specificities, dipeptidyl peptidase, and E-cadherin.

B. Protein Transduction Domains and Fusogenic Peptides

Other functional elements that can be associated with, linked, conjugated, or otherwise attached directly or indirectly to the gene editing molecule, potentiating agent, or to a nanoparticle or other delivery vehicle thereof, include protein transduction domains and fusogenic peptides.

For example, the efficiency of nanoparticle delivery systems can also be improved by the attachment of functional ligands to the NP surface. Potential ligands include, but are not limited to, small molecules, cell-penetrating peptides (CPPs), targeting peptides, antibodies or aptamers (Yu, et al., PLoS One., 6:e24077 (2011), Cu, et al., J Control Release, 156:258-264 (2011), Nie, et al., J Control Release, 138:64-70 (2009), Cruz, et al., J Control Release, 144:118-126 (2010)). Attachment of these moieties serves a variety of different functions; such as inducing intracellular uptake, endosome disruption, and delivery of the plasmid payload to the nucleus. There have been numerous methods employed to tether ligands to the particle surface. One approach is direct covalent attachment to the functional groups on PLGA NPs (Bertram, Acta Biomater. 5:2860-2871 (2009)). Another approach utilizes amphiphilic conjugates like avidin palmitate to secure biotinylated ligands to the NP surface (Fahmy, et al., Biomaterials, 26:5727-5736 (2005), Cu, et al., Nanomedicine, 6:334-343 (2010)). This approach produces particles with enhanced uptake into cells, but reduced pDNA release and gene transfection, which is likely due to the surface modification occluding pDNA release. In a similar approach, lipid-conjugated polyethylene glycol (PEG) is used as a multivalent linker of penetratin, a CPP, or folate (Cheng, et al., Biomaterials, 32:6194-6203 (2011)).

These methods, as well as other methods discussed herein, and others methods known in the art, can be combined to tune particle function and efficacy. In some preferred embodiments, PEG is used as a linker for linking functional molecules to nanoparticles. For example, DSPE-PEG(2000)-maleimide is commercially available and can be used utilized for covalently attaching functional molecules such as CPP.

“Protein Transduction Domain” or PTD refers to a polypeptide, polynucleotide, or organic or inorganic compounds that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. PTA can be short basic peptide sequences such as those present in many cellular and viral proteins. Exemplary protein transduction domains that are well-known in the art include, but are not limited to, the Antennapedia PTD and the TAT (transactivator of transcription) PTD, poly-arginine, poly-lysine or mixtures of arginine and lysine, HIV TAT (YGRKKRRQRRR (SEQ ID NO:7) or RKKRRQRRR (SEQ ID NO:8), 11 arginine residues, VP22 peptide, and an ANTp peptide (RQIKIWFQNRRMKWKK) (SEQ ID NO:9) or positively charged polypeptides or polynucleotides having 8-15 residues, preferably 9-11 residues. Short, non-peptide polymers that are rich in amines or guanidinium groups are also capable of carrying molecules crossing biological membranes. Penetratin and other derivatives of peptides derived from antennapedia (Cheng, et al., Biomaterials, 32(26):6194-203 (2011) can also be used. Results show that penetratin in which additional Args are added, further enhances uptake and endosomal escape, and IKK NBD, which has an antennapedia domain for permeation as well as a domain that blocks activation of NFkB and has been used safely in the lung for other purposes (von Bismarck, et al., Pulmonary Pharmacology & Therapeutics, 25(3):228-35 (2012), Kamei, et al., Journal Of Pharmaceutical Sciences, 102(11):3998-4008 (2013)).

A “fusogenic peptide” is any peptide with membrane destabilizing abilities. In general, fusogenic peptides have the propensity to form an amphiphilic alpha-helical structure when in the presence of a hydrophobic surface such as a membrane. The presence of a fusogenic peptide induces formation of pores in the cell membrane by disruption of the ordered packing of the membrane phospholipids. Some fusogenic peptides act to promote lipid disorder and in this way enhance the chance of merging or fusing of proximally positioned membranes of two membrane enveloped particles of various nature (e.g. cells, enveloped viruses, liposomes). Other fusogenic peptides may simultaneously attach to two membranes, causing merging of the membranes and promoting their fusion into one. Examples of fusogenic peptides include a fusion peptide from a viral envelope protein ectodomain, a membrane-destabilizing peptide of a viral envelope protein membrane-proximal domain from the cytoplasmic tails.

Other fusogenic peptides often also contain an amphiphilic-region. Examples of amphiphilic-region containing peptides include: melittin, magainins, the cytoplasmic tail of HIV1 gp41, microbial and reptilian cytotoxic peptides such as bomolitin 1, pardaxin, mastoparan, crabrolin, cecropin, entamoeba, and staphylococcal .alpha.-toxin; viral fusion peptides from (1) regions at the N terminus of the transmembrane (TM) domains of viral envelope proteins, e.g. HIV-1, SIV, influenza, polio, rhinovirus, and coxsackie virus; (2) regions internal to the TM ectodomain, e.g. semliki forest virus, sindbis virus, rota virus, rubella virus and the fusion peptide from sperm protein PH-30: (3) regions membrane-proximal to the cytoplasmic side of viral envelope proteins e.g. in viruses of avian leukosis (ALV), Feline immunodeficiency (FIV), Rous Sarcoma (RSV), Moloney murine leukemia virus (MoMuLV), and spleen necrosis (SNV).

In particular embodiments, a functional molecule such as a CPP is covalently linked to DSPE-PEG-maleimide functionalized nanoparticles such as PBAE/PLGA blended particles using known methods such as those described in Fields, et al., J Control Release, 164(1):41-48 (2012). For example, DSPE-PEG-function molecule can be added to the 5.0% PVA solution during formation of the second emulsion. In some embodiments, the loading ratio is about 5 nmol/mg ligand-to-polymer ratio.

In some embodiments, the functional molecule is a CPP such as those above, or mTAT (HIV-1 (with histidine modification) HHHHRKKRRQRRRRHHHHH (SEQ ID NO:10) (Yamano, et al., J Control Release, 152:278-285 (2011)); or bPrPp (Bovine prion) MVKSKIGSWILVLFVAMWS DVGLCKKRPKP (SEQ ID NO:11) (Magzoub, et al., Biochem Biophys Res Commun., 348:379-385 (2006)); or MPG (Synthetic chimera: SV40 Lg T. Ant.+HIV gb41 coat) GALFLGFLGAAGSTMGAWS QPKKKRKV (SEQ ID NO:12) (Endoh, et al., Adv Drug Deliv Rev., 61:704-709 (2009)).

### VIII. Methods of Manufacture

A. Methods of Making Nanoparticles

The nanoparticle compositions described herein can be prepared by a variety of methods.

1. Polycations

In some embodiments, the nucleic acid is first complexed to a polycation. Complexation can be achieved by mixing the nucleic acids and polycations at an appropriate molar ratio. When a polyamine is used as the polycation species, it is useful to determine the molar ratio of the polyamine nitrogen to the polynucleotide phosphate (N/P ratio). In a preferred embodiment, nucleic acids and polyamines are mixed together to form a complex at an N/P ratio of between approximately 8:1 to 15:1. The volume of polyamine solution required to achieve particular molar ratios can be determined according to the following formula:

$${V}_{NH 2} = \frac{\begin{matrix}{C}_{nucacid , final} \times {M}_{w , nucacid} / {C}_{nucacid , final} \times \\ {M}_{w , P} \times {M}_{w , P} \times {Φ}_{N : P} \times Φ {V}_{final}\end{matrix}}{{C}_{NH 2} / {M}_{w , NH 2}}$$

where M_{w, nucacid}=molecular weight of nucleic acid, M_{w,P}=molecular weight of phosphate groups of the nucleic acid, Φ_{N:P}=N:P ratio (molar ratio of nitrogens from polyamine to the ratio of phosphates from the nucleic acid), C_{NH2}, stock=concentration of polyamine stock solution, and M_{w,NH2}=molecular weight per nitrogen of polyamine.

Polycation complexation with nucleic acids can be achieved by mixing solutions containing polycations with solutions containing nucleic acids. The mixing can occur at any appropriate temperature. In one embodiment, the mixing occurs at room temperature. The mixing can occur with mild agitation, such as can be achieved through the use of a rotary shaker.

2. Exemplary Preferred Methods of Manufacture

In preferred embodiments, the nanoparticles are formed by a double-emulsion solvent evaporation technique, such as is disclosed in U.S. Published Application No. 2011/0008451 or U.S. Published Application No. 2011/0268810, each of which is a specifically incorporated by reference in its entirety, or Fahmy, et al., Biomaterials, 26:5727-5736, (2005), or McNeer, et al., Mol. Ther. 19, 172-180 (2011)). In this technique, the nucleic acids or nucleic acid/polycation complexes are reconstituted in an aqueous solution. Nucleic acid and polycation amounts are discussed in more detail below and can be chosen, for example, based on amounts and ratios disclosed in U.S. Published Application No. 2011/0008451 or U.S. Published Application No. 2011/0268810, or used by McNeer, et al., (McNeer, et al., Mol. Ther. 19, 172-180 (2011)), or by Woodrow et al. for small interfering RNA encapsulation (Woodrow, et al., Nat Mater, 8:526-533 (2009)). This aqueous solution is then added dropwise to a polymer solution of a desired polymer dissolved in an organic solvent to form the first emulsion.

This mixture is then added dropwise to solution containing a surfactant, such as polyvinyl alcohol (PVA) and sonicated to form the double emulsion. The final emulsion is then poured into a solution containing the surfactant in an aqueous solution and stirred for a period of time to allow the dichloromethane to evaporate and the particles to harden. The concentration of the surfactant used to form the emulsion, and the sonication time and amplitude can been optimized according to principles known in the art for formulating particles with a desired diameter. The particles can be collected by centrifugation. If it is desirable to store the nanoparticles for later use, they can be rapidly frozen, and lyophilized.

In preferred embodiments the nanoparticles are PLGA nanoparticles. In a particular exemplary protocol, nucleic acid (such as PNA, DNA, or PNA-DNA) with or without a polycation (such as spermidine) are dissolved in DNAse/RNAse free H₂O. Encapsulant in H₂O can be added dropwise to a polymer solution of 50:50 ester-terminated PLGA dissolved in dichloromethane (DCM), then sonicated to form the first emulsion. This emulsion can then be added dropwise to 5% polyvinyl alcohol, then sonicated to form the second emulsion. This mixture can be poured into 0.3% polyvinyl alcohol, and stirred at room temperature to form nanoparticles. Nanoparticles can then be collected and washed with, for example H₂O, collected by centrifugation, and then resuspended in H₂O, frozen at −80° C., and lyophilized. Particles can be stored at −20° C. following lyophilization.

Additional techniques for encapsulating the nucleic acid and polycation complex into polymeric nanoparticles are described below.

3. Solvent Evaporation

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid particles. The resulting particles are washed with water and dried overnight in a lyophilizer. Particles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

4. Interfacial Polycondensation

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

5. Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer such as PLA, PLA/PGA copolymer, or PLA/PCL copolymer microcapsules. The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

Solvent evaporation microencapsulation can result in the stabilization of insoluble active agent particles in a polymeric solution for a period of time ranging from 0.5 hours to several months. Stabilizing an insoluble pigment and polymer within the dispersed phase (typically a volatile organic solvent) can be useful for most methods of microencapsulation that are dependent on a dispersed phase, including film casting, solvent evaporation, solvent removal, spray drying, phase inversion, and many others.

The stabilization of insoluble active agent particles within the polymeric solution could be critical during scale-up. By stabilizing suspended active agent particles within the dispersed phase, the particles can remain homogeneously dispersed throughout the polymeric solution as well as the resulting polymer matrix that forms during the process of microencapsulation.

Solvent evaporation microencapsulation (SEM) have several advantages. SEM allows for the determination of the best polymer-solvent-insoluble particle mixture that will aid in the formation of a homogeneous suspension that can be used to encapsulate the particles. SEM stabilizes the insoluble particles or pigments within the polymeric solution, which will help during scale-up because one will be able to let suspensions of insoluble particles or pigments sit for long periods of time, making the process less time-dependent and less labor intensive. SEM allows for the creation of nanoparticles that have a more optimized release of the encapsulated material.

6. Hot Melt Microencapsulation

In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between 0.5 to 1000 microns are obtained with this method. The external surfaces of spheres prepared with this technique are usually smooth and dense. This procedure is used to prepare particles made of polyesters and polyanhydrides. However, this method is limited to polymers with molecular weights between 1,000-50,000.

7. Solvent Removal Microencapsulation

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

8. Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

9. Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

10. Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

11. Solvent Removal

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

12. Spray-Drying

In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24o C, outlet temperature=13-15° C., aspirator setting=15, pump setting=10 mL/minute, spray flow=600 Nl/hr, and nozzle diameter=0.5 mm. Particles ranging between 1-10 microns are obtained with a morphology which depends on the type of polymer used.

13. Nanoprecipitation

In nanoprecipitation, the polymer and nucleic acids are co-dissolved in a selected, water-miscible solvent, for example DMSO, acetone, ethanol, acetone, etc. In a preferred embodiment, nucleic acids and polymer are dissolved in DMSO. The solvent containing the polymer and nucleic acids is then drop-wise added to an excess volume of stirring aqueous phase containing a stabilizer (e.g., poloxamer, Pluronic®, and other stabilizers known in the art). Particles are formed and precipitated during solvent evaporation. To reduce the loss of polymer, the viscosity of the aqueous phase can be increased by using a higher concentration of the stabilizer or other thickening agents such as glycerol and others known in the art. Lastly, the entire dispersed system is centrifuged, and the nucleic acid-loaded polymer nanoparticles are collected and optionally filtered. Nanoprecipitation-based techniques are discussed in, for example, U.S. Pat. No. 5,118,528.

Advantages to nanoprecipitation include: the method can significantly increase the encapsulation efficiency of drugs that are polar yet water-insoluble, compared to single or double emulsion methods (Alshamsan, Saudi Pharmaceutical Journal, 22(3):219-222 (2014)). No emulsification or high shear force step (e.g., sonication or high-speed homogenization) is involved in nanoprecipitation, therefore preserving the conformation of nucleic acids. Nanoprecipitation relies on the differences in the interfacial tension between the solvent and the nonsolvent, rather than shear stress, to produce nanoparticles. Hydrophobicity of the drug will retain it in the instantly-precipitating nanoparticles; the un-precipitated polymer due to equilibrium is “lost” and not in the precipitated nanoparticle form.

B. Molecules to be Encapsulated or Attached to the Surface of the Particles

There are two principle groups of molecules to be encapsulated or attached to the polymer, either directly or via a coupling molecule: targeting molecules, attachment molecules and therapeutic, nutritional, diagnostic or prophylactic agents. These can be coupled using standard techniques. The targeting molecule or therapeutic molecule to be delivered can be coupled directly to the polymer or to a material such as a fatty acid which is incorporated into the polymer.

Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways. The first is during the preparation of the particles, for example during the emulsion preparation of particles by incorporation of stabilizers with functional chemical groups. Example 1 demonstrates this type of process whereby functional amphiphilic molecules are inserted into the particles during emulsion preparation.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

In the preferred embodiment, the surface is modified to insert amphiphilic polymers or surfactants that match the polymer phase HLB or hydrophile-lipophile balance, as demonstrated in the following example. HLBs range from 1 to 15. Surfactants with a low HLB are more lipid loving and thus tend to make a water in oil emulsion while those with a high HLB are more hydrophilic and tend to make an oil in water emulsion. Fatty acids and lipids have a low HLB below 10. After conjugation with target group (such as hydrophilic avidin), HLB increases above 10. This conjugate is used in emulsion preparation. Any amphiphilic polymer with an HLB in the range 1-10, more preferably between 1 and 6, most preferably between 1 and up to 5, can be used. This includes all lipids, fatty acids and detergents.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of molecules to the polymer.

Coupling is preferably by covalent binding but it may also be indirect, for example, through a linker bound to the polymer or through an interaction between two molecules such as strepavidin and biotin. It may also be by electrostatic attraction by dip-coating.

The molecules to be delivered can also be encapsulated into the polymer using double emulsion solvent evaporation techniques, such as that described by Luo et al., Controlled DNA delivery system, Phar. Res., 16: 1300-1308 (1999).

C. Particularly Preferred Nanoparticle Formulations

The nanoparticle formulation can be selected based on the considerations including the targeted tissue or cells. For example, in embodiments directed to treatment of treating or correcting beta-thalassemia (e.g. when the target cells are, for example, hematopoietic stem cells), a preferred nanoparticle formulation is PLGA.

Other preferred nanoparticle formulations, particularly preferred for treating cystic fibrosis, are described in McNeer, et al., Nature Commun., 6:6952. doi: 10.1038/ncomms7952 (2015), and Fields, et al., Adv Healthc Mater., 4(3):361-6 (2015). doi: 10.1002/adhm.201400355 (2015) Epub 2014. Such nanoparticles are composed of a blend of Poly(beta-amino) esters (PBAEs) and poly(lactic-co-glycolic acid) (PLGA). Poly(beta-amino) esters (PBAEs) are degradable, cationic polymers synthesized by conjugate (Michael-like) addition of bifunctional amines to diacrylate esters (Lynn, Langer R, editor. J Am Chem Soc. 2000. pp. 10761-10768). PBAEs appear to have properties that make them efficient vectors for gene delivery. These cationic polymers are able to condense negatively charged pDNA, induce cellular uptake, and buffer the low pH environment of endosomes leading to DNA escape (Lynn, Langer R, editor. J Am Chem Soc. 2000. pp. 10761-10768, and Green, Acc Chem Res., 41(6):749-759 (2008)). PBAEs have the ability to form hybrid particles with other polymers, which allows for production of solid, stable and storable particles. For example, blending cationic PBAE with PLGA produced highly loaded pDNA particles. The addition of PBAE to PLGA resulted in an increase in gene transfection in vitro and induced antigen-specific tumor rejection in a murine model (Little, et al. Proc Natl Acad Sci USA., 101:9534-9539 (2004), Little, et al., J Control Release, 107:449-462 (2005)).

Therefore, in some embodiments, the nanoparticles utilized to deliver the disclosed compositions are composed of a blend of PBAE and a second polymer one of those discussed above. In some embodiments, the nanoparticles are composed of a blend of PBAE and PLGA.

PLGA and PBAE/PLGA blended nanoparticles loaded with gene editing technology can be formulated using a double-emulsion solvent evaporation technique such as that described in detail above, and in McNeer, et al., Nature Commun., 6:6952. doi: 10.1038/ncomms7952 (2015), and Fields, et al., Adv Healthc Mater., 4(3):361-6 (2015). doi: 10.1002/adhm.201400355 (2015) Epub 2014. Poly(beta amino ester) (PBAE) can synthesized by a Michael addition reaction of 1,4-butanediol diacrylate and 4,4′-trimethylenedipiperidine as described in Akinc, et al., Bioconjug Chem., 14:979-988 (2003). In some embodiments, PBAE blended particles such as PLGA/PBAE blended particles, contain between about 1 and 99, or between about 1 and 50, or between about 5 and 25, or between about 5 and 20, or between about 10 and 20, or about 15 percent PBAE (wt %). In particular embodiments, PBAE blended particles such as PLGA/PBAE blended particles, contain about 50, 45, 40, 35, 30, 25, 20, 15, 10, or 5% PBAE (wt %). Solvent from these particles in PVA as discussed above, and in some cases may continue overnight. PLGA/PBAE/MPG nanoparticles was shown to produce significantly greater nanoparticle association with airway epithelial cells than PLGA nanoparticles (Fields, et al., Advanced Healthcare Materials, 4:361-366 (2015)).

### IX. Methods of Use

A. Methods of Treatment

The disclosed compositions can be used to ex vivo or in vivo gene editing. The methods typically include contacting a cell with an effective amount of gene editing composition, preferably in combination with a potentiating agent, to modify the cell's genome. As discussed in more detail below, the contacting can occur ex vivo or in vivo. In preferred embodiments, the method includes contacting a population of target cells with an effective amount of gene editing composition, preferably in combination with a potentiating agent, to modify the genomes of a sufficient number of cells to achieve a therapeutic result.

For example, the effective amount or therapeutically effective amount can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder.

In some embodiments, when the gene editing technology is triplex forming molecules, the molecules can be administered in an effective amount to induce formation of a triple helix at the target site. An effective amount of gene editing technology such as triplex-forming molecules may also be an amount effective to increase the rate of recombination of a donor fragment relative to administration of the donor fragment in the absence of the gene editing technology. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids. The precise dosage will vary according to a variety of factors such as subject-dependent variables (e.g., age, immune system health, clinical symptoms etc.). Exemplary symptoms, pharmacologic, and physiologic effects are discussed in more detail below.

The disclosed compositions can be administered or otherwise contacted with target cells once, twice, or three time daily; one, two, three, four, five, six, seven times a week, one, two, three, four, five, six, seven or eight times a month. For example, in some embodiments, the composition is administered every two or three days, or on average about 2 to about 4 times about week.

In some embodiments, the potentiating agent is administered to the subject prior to administration of the gene editing technology to the subject. The potentiating agent can be administered to the subject, for example, 1, 2, 3, 4, 5, 6, 8, 10, 12, 18, or 24 hours, or 1, 2, 3, 4, 5, 6, or 7 days, or any combination thereof prior to administration of the gene editing technology to the subject.

In some embodiments, the gene editing technology is administered to the subject prior to administration of the potentiating agent to the subject. The gene editing technology can be administered to the subject, for example, 1, 2, 3, 4, 5, 6, 8, 10, 12, 18, or 24 hours, or 1, 2, 3, 4, 5, 6, or 7 days, or any combination thereof prior to administration of the potentiating agent to the subject.

In preferred embodiments, the compositions are administered in an amount effective to induce gene modification in at least one target allele to occur at frequency of at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25% of target cells. In some embodiments, particularly ex vivo applications, gene modification occurs in at least one target allele at a frequency of about 0.1-25%, or 0.5-25%, or 1-25% 2-25%, or 3-25%, or 4-25% or 5-25% or 6-25%, or 7-25%, or 8-25%, or 9-25%, or 10-25%, 11-25%, or 12-25%, or 13%-25% or 14%-25% or 15-25%, or 2-20%, or 3-20%, or 4-20% or 5-20% or 6-20%, or 7-20%, or 8-20%, or 9-20%, or 10-20%, 11-20%, or 12-20%, or 13%-20% or 14%-20% or 15-20%, 2-15%, or 3-15%, or 4-15% or 5-15% or 6-15%, or 7-15%, or 8-15%, or 9-15%, or 10-15%, 11-15%, or 12-15%, or 13%-15% or 14%-15%.

In some embodiments, particularly in vivo applications, gene modification occurs in at least one target allele at a frequency of about 0.1% to about 10%, or about 0.2% to about 10%, or about 0.3% to about 10%, or about 0.4% to about 10%, or about 0.5% to about 10%, or about 0.6% to about 10%, or about 0.7% to about 10%, or about 0.8% to about 10%, or about 0.9% to about 10%, or about 1.0% to about 10%, or about 1.1% to about 10%, or about 1.1% to about 10%, 1.2% to about 10%, or about 1.3% to about 10%, or about 1.4% to about 10%, or about 1.5% to about 10%, or about 1.6% to about 10%, or about 1.7% to about 10%, or about 1.8% to about 10%, or about 1.9% to about 10%, or about 2.0% to about 10%, or about 2.5% to about 10%, or about 3.0% to about 10%, or about 3.5% to about 10%, or about 4.0% to about 10%, or about 4.5% to about 10%, or about 5.0% to about 10%.

In some embodiments, gene modification occurs with low off-target effects. In some embodiments, off-target modification is undetectable using routine analysis such as those described in the Examples below. In some embodiments, off-target incidents occur at a frequency of 0-1%, or 0-0.1%, or 0-0.01%, or 0-0.001%, or 0-0.0001%, or 0-0000.1%, or 0-0.000001%. In some embodiments, off-target modification occurs at a frequency that is about 10², 10³, 10⁴, or 10⁵-fold lower than at the target site.

Gene Editing Technology

In general, by way of example only, dosage forms useful in the disclosed methods can include doses in the range of about 10² to about 10⁵⁰, or about 10⁵ to about 10⁴⁰, or about 10¹⁰ to about 10³⁰, or about 10¹² to about 10²⁰ copies of the gene editing technology per dose. In particular embodiments, about 10¹³, 10¹⁴, 10¹⁵, 10¹⁶, or 10¹⁷ copies of gene editing technology are administered to a subject in need thereof.

In other embodiments, dosages are expressed in moles. For example, in some embodiments, the dose of gene editing technology is about 0.1 nmol to about 100 nmol, or about 0.25 nmol to about 50 nmol, or about 0.5 nmol to about 25 nmol, or about 0.75 nmol to about 7.5 nmol.

In other embodiments, dosages are expressed in molecules per target cells. For example, in some embodiments, the dose of gene editing technology is about 10² to about 10⁵⁰, or about 10⁵ to about 10¹⁵, or about 10⁷ to about 10¹², or about 10⁸ to about 10¹¹ copies of the gene editing technology per target cell.

In other embodiments, dosages are expressed in mg/kg, particularly when the expressed as an in vivo dosage of gene editing composition packaged in a nanoparticle with or without functional molecules. Dosages can be, for example 0.1 mg/kg to about 1,000 mg/kg, or 0.5 mg/kg to about 1,000 mg/kg, or 1 mg/kg to about 1,000 mg/kg, or about 10 mg/kg to about 500 mg/kg, or about 20 mg/kg to about 500 mg/kg per dose, or 20 mg/kg to about 100 mg/kg per dose, or 25 mg/kg to about 75 mg/kg per dose, or about 25, 30, 35, 40, 45, 50, 55, 60, 65, 70, or 75 mg/kg per dose.

In other embodiments, dosages are expressed in mg/ml, particularly when the expressed as an ex vivo dosage of gene editing composition packaged in a nanoparticle with or without functional molecules. Dosages can be, for example 0.01 mg/ml to about 100 mg/ml, or about 0.5 mg/ml to about 50 mg/ml, or about 1 mg/ml to about 10 mg/ml per dose to a cell population of 10⁶ cells.

As discussed above, gene editing technology can be administered without, but is preferably administered with at least one donor oligonucleotide. Such donors can be administered at similar dosages as the gene editing technology. Compositions should include an amount of donor fragment effective to recombine at the target site in the presence of a gene editing technology such as triplex forming molecules.

Potentiating Agents

The methods can include contacting cells with an effective amount potentiating agents. Preferably the amount of potentiating agent is effective to increase gene modification when used in combination with a gene modifying technology, compared to using the gene modifying technology in the absence of the potentiating agent.

Exemplary dosages for SCF include, about 0.01 mg/kg to about 250 mg/kg, or about 0.1 mg/kg to about 100 mg/kg, or about 0.5 mg/kg to about 50 mg/kg, or about 0.75 mg/kg to about 10 mg/kg.

Dosages for CHK1 inhibitors are known in the art, and many of these are in clinical trial. Accordingly, the dosage can be selected by the practitioner based on known, preferred humans dosages. In preferred embodiments, the dosage is below the lowest-observed-adverse-effect level (LOAEL), and is preferably a no observed adverse effect level (NOAEL) dosage.

1. Ex Vivo Gene Therapy

In some embodiments, ex vivo gene therapy of cells is used for the treatment of a genetic disorder in a subject. For ex vivo gene therapy, cells are isolated from a subject and contacted ex vivo with the compositions to produce cells containing mutations in or adjacent to genes. In a preferred embodiment, the cells are isolated from the subject to be treated or from a syngenic host. Target cells are removed from a subject prior to contacting with a gene editing composition and preferably a potentiating factor. The cells can be hematopoietic progenitor or stem cells. In a preferred embodiment, the target cells are CD34⁺ hematopoietic stem cells. Hematopoietic stem cells (HSCs), such as CD34+ cells are multipotent stem cells that give rise to all the blood cell types including erythrocytes. Therefore, CD34+ cells can be isolated from a patient with, for example, thalassemia, sickle cell disease, or a lysosomal storage disease, the mutant gene altered or repaired ex-vivo using the disclosed compositions and methods, and the cells reintroduced back into the patient as a treatment or a cure.

Stem cells can be isolated and enriched by one of skill in the art. Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 deposited with the American Type Culture Collection (Rockville, Md.) as HB-8483 is commercially available as anti-HPCA 1. Additionally, negative selection of differentiated and “dedicated” cells from human bone marrow can be utilized, to select against substantially any desired cell marker. For example, progenitor or stem cells, most preferably CD34⁺ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium including cell surface factors supporting the proliferation of stem cells. Stromal cells may be freed of hematopoietic cells employing appropriate monoclonal antibodies for removal of the undesired cells.

The isolated cells are contacted ex vivo with a combination of triplex-forming molecules and donor oligonucleotides in amounts effective to cause the desired mutations in or adjacent to genes in need of repair or alteration, for example the human beta-globin or a-L-iduronidase gene. These cells are referred to herein as modified cells. Methods for transfection of cells with oligonucleotides and peptide nucleic acids are well known in the art (Koppelhus, et al., Adv. Drug Deliv. Rev., 55(2): 267-280 (2003)). It may be desirable to synchronize the cells in S-phase to further increase the frequency of gene correction. Methods for synchronizing cultured cells, for example, by double thymidine block, are known in the art (Zielke, et al., Methods Cell Biol., 8:107-121 (1974)).

The modified cells can be maintained or expanded in culture prior to administration to a subject. Culture conditions are generally known in the art depending on the cell type. Conditions for the maintenance of CD34⁺ in particular have been well studied, and several suitable methods are available. A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) including murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the invention as well. Cells can also be grown in serum-free medium, as described in U.S. Pat. No. 5,945,337.

In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with triplex-forming molecules and donor oligonucleotides as described above with respect to CD34⁺ cells to produce recombinant cells having one or more modified genes. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes weeks to months.

A high percentage of engraftment of modified hematopoietic stem cells is not envisioned to be necessary to achieve significant prophylactic or therapeutic effect. It is expected that the engrafted cells will expand over time following engraftment to increase the percentage of modified cells. In some embodiments, the modified cells have a corrected a-L-iduronidase gene. Therefore, in a subject with Hurler syndrome, the modified cells are expected to improve or cure the condition. It is expected that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic.

2. In Vivo Gene Therapy

The disclosed compositions can be administered directly to a subject for in vivo gene therapy.

a. Pharmaceutical Formulations

The disclosed compositions are preferably employed for therapeutic uses in combination with a suitable pharmaceutical carrier. Such compositions include an effective amount of the composition, and a pharmaceutically acceptable carrier or excipient.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions including triplex-forming molecules, such as TFOs and PNAs, and donor fragments may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example, in Sambrook et al., Molecular Cloning: A Laboratory Manual, Cold Spring Harbor Laboratory, New York (1989); and Ausubel et al., Current Protocols in Molecular Biology, John Wiley & Sons, New York (1994). Such nucleic acid delivery systems include the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the triplex-forming molecules and/or donor oligonucleotides are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

b. Methods of Administration

In general, methods of administering compounds, including oligonucleotides and related molecules, are well known in the art. In particular, the routes of administration already in use for nucleic acid therapeutics, along with formulations in current use, provide preferred routes of administration and formulation for the triplex-forming molecules described above. Preferably the compositions are injected into the organism undergoing genetic manipulation, such as an animal requiring gene therapy.

The disclosed compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the gene editing compositions to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The compositions may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the triplex-forming molecules and donor oligonucleotides. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

c. Preferred Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

C. Diseases to be Treated

Gene therapy is apparent when studied in the context of human genetic diseases, for example, cystic fibrosis, hemophilia, globinopathies such as sickle cell anemia and beta-thalassemia, xeroderma pigmentosum, and lysosomal storage diseases, though the strategies are also useful for treating non-genetic disease such as HIV, in the context of ex vivo-based cell modification and also for in vivo cell modification. The disclosed compositions are especially useful to treat genetic deficiencies, disorders and diseases caused by mutations in single genes, for example, to correct genetic deficiencies, disorders and diseases caused by point mutations. If the target gene contains a mutation that is the cause of a genetic disorder, then the disclosed compositions can be used for mutagenic repair that may restore the DNA sequence of the target gene to normal. The target sequence can be within the coding DNA sequence of the gene or within an intron. The target sequence can also be within DNA sequences that regulate expression of the target gene, including promoter or enhancer sequences.

If the target gene is an oncogene causing unregulated proliferation, such as in a cancer cell, then the oligonucleotide is useful for causing a mutation that inactivates the gene and terminates or reduces the uncontrolled proliferation of the cell. The oligonucleotide is also a useful anti-cancer agent for activating a repressor gene that has lost its ability to repress proliferation. The target gene can also be a gene that encodes an immune regulatory factor, such as PD-1, in order to enhance the host's immune response to a cancer.

Programmed cell death protein 1, also known as PD-1 and CD279 (cluster of differentiation 279), is a protein encoded by the PDCD1 gene. PD-1 has two ligands: PD-L1 and PD-L2. PD-1 is expressed on a subset of thymocytes and up-regulated on T, B, and myeloid cells after activation (Agata, et al., Int. Immunol., 8:765-772 (1996)). PD-1 acts to antagonize signal transduction downstream of the TCR after it binds a peptide antigen presented by the major histocompatibility complex (MHC). It can function as an immune checkpoint, by preventing the activation of T-cells, which in turn reduces autoimmunity and promotes self-tolerance, but can also reduce the body's ability to combat cancer. The inhibitory effect of PD-1 to act through twofold mechanism of promoting apoptosis (programmed cell death) in antigen specific T-cells in lymph nodes while simultaneously reducing apoptosis in regulatory T cells (suppressor T cells). Compositions that block PD-1, the PD-1 inhibitors, activate the immune system to attack tumors and are therefore used with varying success to treat some types of cancer.

Therefore, in some embodiments, compositions are used to treat cancer. The gene modification technology can be designed to reduce or prevent expression of PD-1, and administered in an effective amount to do so.

The compositions can be used as antiviral agents, for example, when designed to modify a specific a portion of a viral genome necessary for proper proliferation or function of the virus.

### Variants, Substitutions, and Exemplary PNAs

Preferred diseases and sequences of exemplary targeting sites, triplex forming molecules, and donor oligonucleotides are discussed in more detail below. Any of the sequences can also be modified as disclosed herein or otherwise known in the art. For example, in some embodiments, any of the triplex-forming sequences herein can have one or more mutations (e.g., substitutions, deletions, or insertions), such that the triplex-forming molecules still bind to the target sequence.

Any of the triplex-forming sequences herein can be manufactured using canonical nucleic acids or other suitable substitutes including those disclosed herein (e.g., PNAs), without or without any of the base, sugar, or backbone modifications discussed herein or in WO 1996/040271, WO/2010/123983, and U.S. Pat. No. 8,658,608.

Any of the triplex-forming sequences herein can be peptide nucleic acids. In some embodiments, one or more of the cytosines of any of triplex-forming sequences herein is substituted with a pseudoisocytosine. In some embodiments, all of the cytosines in the Hoogsteen-binding portion of a triplex forming molecule are substituted with pseudoisocytosine. In some embodiments, any of the triplex-forming sequences herein, includes one or more of peptide nucleic acid monomers substituted with a γPNA. In some embodiments all of the peptide nucleic acid monomers in the Hoogsteen-binding portion only, the Watson-Crick-binding portion only, or across the entire PNA are substituted with γPNA monomers. In particular embodiments, alternating residues are PNA and γPNA in the Hoogsteen-binding portion only, the Watson-Crick-binding portion only, or across the entire PNA are substituted. In some embodiments, the γPNAs are miniPEG γPNA, methyl γPNA, another γ substitution discussed above. In some embodiments, the PNA oligomer includes two or more different γPNAs.

For example, in some embodiments, (1) some or all of the residues in the Watson-Crick binding portion are γPNA (e.g., miniPEG-containing γPNA); (2) some or all of the residues in the Hoogsteen binding portion are γPNA (e.g., miniPEG-containing γPNA); or (3) some or all of the residue (in the Watson-Crick and/or Hoogsteen binding portions) are γPNA (e.g., miniPEG-containing γPNA). Therefore, in some embodiments any of the triplex forming nucleic acid sequence herein is a peptide nucleic acid wherein (1) all of the residues in the Watson-Crick binding portion are γPNA (e.g., miniPEG-containing γPNA) and none of the residues is in Hoogsteen binding portion are γPNA (e.g., miniPEG-containing γPNA); (2) all of the residues in the Hoogsteen binding portion are γPNA (e.g., miniPEG-containing γPNA) and none of the residues is in Watson-Crick binding portion are γPNA (e.g., miniPEG-containing γPNA); or (3) all of the residues (in the Watson-Crick and Hoogsteen binding portions) are γPNA (e.g., miniPEG-containing γPNA).

Preferred triplex molecules are bis-peptide nucleic acids with pseudoisocytosine substituted for one or more cytosines, particularly in the Hoogsteen-binding portion, and wherein some or all of the PNA are γPNA.

Any of the triplex-forming sequences herein can have one or more G-clamp monomers. For example, one or more cytosines or variant thereof such as pseudoisocytosine in any of the triplex-forming sequences herein can be substituted or otherwise modified to be a clamp-G (9-(2-guanidinoethoxy) phenoxazine).

Any of the triplex-forming sequences herein can include a flexible linker, linking, for example, a Hoogsteen-binding domain and a Watson-Crick binding domain to form a bis-PNA. The sequences can be linked with a flexible linker. For example, in some embodiments the flexible linker includes about 1-10, more preferably 2-5, most preferably about 3 units such as 8-amino-2, 6, 10-trioxaoctanoic acid residues. Some molecules include N-terminal or C-terminal non-binding residues, preferably positively charged. For example, some molecules include 1-10, preferable 2-5, most preferably about 3 lysines at the N-terminus, the C-terminus, or a combination thereof of the PNA.

For the disclosed sequences, “J” is pseudoisocytosine, “0” is flexible 8-amino-3,6-dioxaoctanoic acid, 6-aminohexanoic acid, or 8-amino-2, 6, 10-trioxaoctanoic acid monomers, “K” and “lys” are lysine. PNA sequences are generally presented in an H-“nucleic acid sequence”-NH₂ orientation. For bis-PNA the Hoosten-binding portion is typically oriented up stream (e.g., at the “H” end) of the linker, while the Watson-Crick-binding portion is typically oriented downstream (e.g., at the NH₂ end) of the linker. Any of the donors can include optional phosphorothioate internucleoside linkages, particular between the three or four terminal 5′ and three or four terminal 3′ nucleotides. Thus, each of the donor oligonucleotide sequences disclosed herein is expressly disclosed without any phosphorothioate internucleoside linkages, and with phosphorothioate internucleoside linkages, preferably between the three or four terminal 5′ and three or four terminal 3′ nucleotides.

1. Globinopathies

Worldwide, globinopathies account for significant morbidity and mortality. Over 1,200 different known genetic mutations affect the DNA sequence of the human alpha-like (HBZ, HBA2, HBA1, and HBQ1) and beta-like (HBE1, HBG1, HBD, and HBB) globin genes. Two of the more prevalent and well-studied globinopathies are sickle cell anemia and β-thalassemia. Substitution of valine for glutamic acid at position 6 of the β-globin chain in patients with sickle cell anemia predisposes to hemoglobin polymerization, leading to sickle cell rigidity and vasoocclusion with resulting tissue and organ damage. In patients with β-thalassemia, a variety of mutational mechanisms results in reduced synthesis of β-globin leading to accumulation of aggregates of unpaired, insoluble α-chains that cause ineffective erythropoiesis, accelerated red cell destruction, and severe anemia.

Together, globinopathies represent the most common single-gene disorders in man. Triplex forming oligonucleotides are particularly well suited to treat globinopathies, as they are single gene disorders caused by point mutations. Triplex forming molecules disclosed herein are effective at binding to the human β-globin both in vitro and in living cells, both ex vivo and in vivo in animals. Experimental results also demonstrate correction of a thalassemia-associated mutation in vivo in a transgenic mouse carrying a human beta globin gene with the IVS2-654 thalassemia mutation (in place of the endogenous mouse beta globin) with correction of the mutation in 4% of the total bone marrow cells, cure of the anemia with blood hemoglobin levels showing a sustained elevation into the normal range, reversal of extramedullary hematopoiesis and reversal of splenomegaly, and reduction in reticulocyte counts, following systemic administration of PNA and DNA containing nanoparticles.

β-thalassemia is an unstable hemoglobinopathy leading to the precipitation of α-hemoglobin within RBCs resulting in a severe hemolytic anemia. Patients experience jaundice and splenomegaly, with substantially decreased blood hemoglobin concentrations necessitating repeated transfusions, typically resulting in severe iron overload with time. Cardiac failure due to myocardial siderosis is a major cause of death from β-thalassemia by the end of the third decade. Reduction of repeated blood transfusions in these patients is therefore of primary importance to improve patient outcomes.

a. Exemplary β-Globin Gene Target Sites

In the β-globin gene sequence, particularly in the introns, there are many good third-strand binding sites that may be utilized in the methods disclosed herein. A portion of the GenBank sequence of the chromosome-11 human-native hemoglobin-gene cluster (GenBank: U01317.1—Human beta globin region on chromosome 11—LOCUS HUMHBB, 73308 bp ds-DNA) from base 60001 to base 66060 is presented below. The start of the gene coding sequence at position 62187-62189 (or positions 2187-2189 of SEQ ID NO: 13) is indicated by wave underlining. This portion of the GenBank sequence contains the native 3 globin gene sequence. In sickle cell hemoglobin the adenine base at position 62206 (or position 2206 as listed in SEQ ID NO:13, indicated in bold and heavy underlining) is mutated to a thymine. Other common point mutations occur in intron 2 (IVS2), which is highlighted in the sequence below by italics (SEQ ID NO: 14) and corresponds with nucleotides 2,632-3,481 of SEQ ID NO:13. Mutations include IVS2-1, IVS2-566, IVS2-654, IVS2-705, and IVS2-745, which are also shown in bold and heavy underlining; numbering relative to the start of intron 2.

Exemplary triplex forming molecule binding sites, are provided in, for example, WO 1996/040271, WO/2010/123983, and U.S. Pat. No. 8,658,608, and in the working Examples below. Target regions can be reference based on the coding strand of genomic DNA, or the complementary non-coding sequence thereto (e.g., the Watson or Crick stand). Exemplary target regions are identified with reference to the coding sequence of the β globin gene sequence in the sequence below by double underlining and a combination of underlining and double underlining (wherein the underlining is optional additional binding sequence). Additionally, for each targeting sequence identified, the complementary target sequence on the reverse non-coding strand is also explicitly disclosed as a triplex forming molecule binding sequence.

Accordingly, triplex forming molecules can be designed to bind a target region on either the coding or non-coding strand. However, as discussed above, triplex-forming molecules, such as PNA and tcPNA preferably invade the target duplex, displacement of the polypyrimidine, and induce triplex formation with the displaced polypurine.

AAAGCTCTTGCTTTGACAATTTTGGTCTTTCAGAATACTATAAATATAACCTATATTATA
ATTTCATAAAGTCTGTGCATTTTCTTTGACCCAGGATATTTGCAAAAGACATATTCAAAC
TTCCGCAGAACACTTTATTTCACATATACATGCCTCTTATATCAGGGATGTGAAACAGGG
TCTTGAAAACTGTCTAAATCTAAAACAATGCTAATGCAGGTTTAAATTTAATAAAATAAA
ATCCAAAATCTAACAGCCAAGTCAAATCTGTATGTTTTAACATTTAAAATATTTTAAAGA
CGTCTTTTCCCAGGATTCAACATGTGAAATCTTTTCTCAGGGATACACGTGTGCCTAGAT
CCTCATTGCTTTAGTTTTTTACAGAGGAATGAATATAAAAAGAAAATACTTAAATTTTAT
CCCTCTTACCTCTATAATCATACATAGGCATAATTTTTTAACCTAGGCTCCAGATAGCCA
TAGAAGAACCAAACACTTTCTGCGTGTGTGAGAATAATCAGAGTGAGATTTTTTCAGAAG
TACCTGATGAGGGTTGAGACAGGTAGAAAAAGTGAGAGATCTCTATTTATTTAGCAATAA
TAGAGAAAGCATTTAAGAGAATAAAGCAATGGAAATAAGAAATTTGTAAATTTCCTTCTG
ATAACTAGAAATAGAGGATCCAGTTTCTTTTGGTTAACCTAAATTTTATTTCATTTTATT
GTTTTATTTTATTTTATTTTATTTTATTTTGTGTAATCGTAGTTTCAGAGTGTTAGAGCT

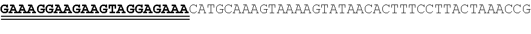

ACTGGGTTTCCAGGTAGGGGCAGGATTCAGGATGACTGACAGGGCCCTTAGGGAACACTG
AGACCCTACGCTGACCTCATAAATGCTTGCTACCTTTGCTGTTTTAATTACATCTTTTAA
TAGCAGGAAGCAGAACTCTGCACTTCAAAAGTTTTTCCTCACCTGAGGAGTTAATTTAGT
ACAAGGGGAAAAAGTACAGGGGGATGGGAGAAAGGCGATCACGTTGGGAAGCTATAGAGA
AAGAAGAGTAAATTTTAGTAAAGGAGGTTTAAACAAACAAAATATAAAGAGAAATAGGAA
CTTGAATCAAGGAAATGATTTTAAAACGCAGTATTCTTAGTGGACTAGAGGAAAAAAATA

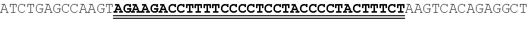

TTTTGTTCCCCCAGACACTCTTGCAGATTAGTCCAGGCAGAAACAGTTAGATGTCCCCAG
TTAACCTCCTATTTGACACCACTGATTACCCCATTGATAGTCACACTTTGGGTTGTAAGT
GACTTTTTATTTATTTGTATTTTTGACTGCATTAAGAGGTCTCTAGTTTTTTATCTCTTG
TTTCCCAAAACCTAATAAGTAACTAATGCACAGAGCACATTGATTTGTAITTATTCTATT
TTTAGACATAATTTATTAGCATGCATGAGCAAATTAAGAAAAACAACAACAAATGAATGC

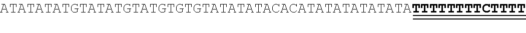

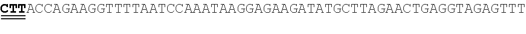

TCATCCATTCTGTCCTGTAAGTATTTTGCATATTCTGGAGACGCAGGAAGAGATCCATCT
ACATATCCCAAAGCTGAATTATGGTAGACAAAGCTCTTCCACTTTTAGTGCATCAATTTC
TTATTTGTGTAATAAGAAAATTGGGAAAACGATCTTCAATATGCTTACCAAGCTGTGATT
CCAAATATTACGTAAATACACTTGCAAAGGAGGATGTTTTTAGTAGCAATTTGTACTGAT
GGTATGGGGCCAAGAGATATATCTTAGAGGGAGGGCTGAGGGTTTGAAGTCCAACTCCTA
AGCCAGTGCCAGAAGAGCCAAGGACAGGTACGGCTGTCATCACTTAGACCTCACCCTGTG
GAGCCACACCCTAGGGTTGGCCAATCTACTCCCAGGAGCAGGGAGGGCAGGAGCCAGGGC
TGGGCATAAAAGTCAGGGCAGAGCCATCTATTGCTTACATTTGCTTCTGACACAACTGTG

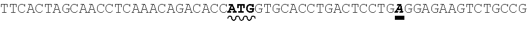

TTACTGCCCTGTGGGGCAAGGTGAACGTGGATGAAGTTGGTGGTGAGGCCCTGGGCAGGT
TGGTATCAAGGTTACAAGACAGGTTTAAGGAGACCAATAGAAACTGGGCATGTGGAGACA
GAGAAGACTCTTGGGTTTCTGATAGGCACTGACTCTCTCTGCCTATTGGTCTATTTTCCC
ACCCTTAGGCTGCTGGTGGTCTACCCTTGGACCCAGAGGTTCTTTGAGTCCTTTGGGGAT
CTGTCCACTCCTGATGCTGTTATGGGCAACCCTAAGGTGAAGGCTCATGGCAAGAAAGTG
CTCGGTGCCTTTAGTGATGGCCTGGCTCACCTGGACAACCTCAAGGGCACCTTTGCCACA

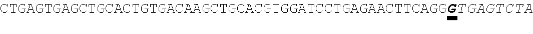

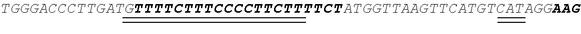

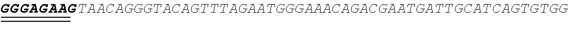

AAGTCTCAGGATCGTTTTAGTTTCTTTTATTTGCTGTTCATAACAATTGTTTTCTTTTGT

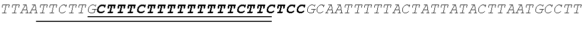

AACATTGTGTATAACAAAAGGAAATATCTCTGAGATACATTAAGTAACTTAAAAAAAAAC
TTTACACAGTCTGCCTAGTACATTACTATTTGGAATATATGTGTGCTTATTTGCATATTC
ATAATCTCCCTACTTTATTTTCTTTTATTTTTAATTGATACATAATCATTATACATATTT
ATGGGTTAAAGTGTAATGTTTTAATATGTGTACACATATTGACCAAATCAGGGTAATTTT

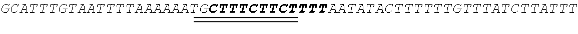

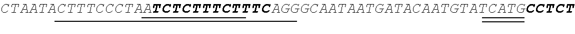

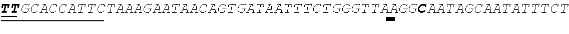

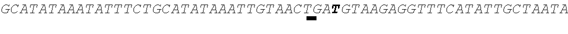

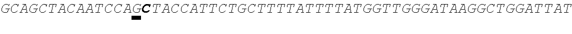

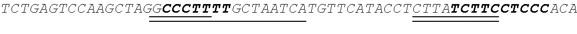

GCTCCTGGGCAACGTGCTGGTCTGTGTGCTGGCCCATCACTTTGGCAAAGAATTCACCCC
ACCAGTGCAGGCTGCCTATCAGAAAGTGGTGGCTGGTGTGGCTAATGCCCTGGCCCACAA
GTATCACTAAGCTCGCTTTCTTGCTGTCCAATTTCTATTAAAGGTTCCTTTGTTCCCTAA
GTCCAACTACTAAACTGGGGGATATTATGAAGGGCCTTGAGCATCTGGATTCTGCCTAAT
AAAAAACATTTATTTTCATTGCAATGATGTATTTAAATTATTTCTGAATATTTTACTAAA
AAGGGAATGTGGGAGGTCAGTGCATTTAAAACATAAAGAAATGAAGAGCTAGTTCAAACC
TTGGGAAAATACACTATATCTTAAACTCCATGAAAGAAGGTGAGGCTGCAAACAGCTAAT
GCACATTGGCAACAGCCCTGATGCCTATGCCTTATTCATCCCTCAGAAAAGGATTCAAGT
AGAGGCTTGATTTGGAGGTTAAAGTTTTGCTATGCTGTATTTTACATTACTTATTGTTTT
AGCTGTCCTCATGAATGTCTTTTCACTACCCATTTGCTTATCCTGCATCTCTCAGCCTTG
ACTCCACTCAGTTCTCTTGCTTAGAGATACCACCTTTCCCCTGAAGTGTTCCTTCCATGT
TTTACGGCGAGATGGTTTCTCCTCGCCTGGCCACTCAGCCTTAGTTGTCTCTGTTGTCTT
ATAGAGGTCTACTTGAAGAAGGAAAAACAGGGGGCATGGTTTGACTGTCCTGTGAGCCCT
TCTTCCCTGCCTCCCCCACTCACAGTGACCCGGAATCTGCAGTGCTAGTCTCCCGGAACT
ATCACTCTTTCACAGTCTGCTTTGGAAGGACTGGGCTTAGTATGAAAAGTTAGGACTGAG
AAGAATTTGAAAGGGGGCTTTTTGTAGCTTGATATTCACTACTGTCTTATTACCCTATCA
TAGGCCCACCCCAAATGGAAGTCCCATTCTTCCTCAGGATGTTTAAGATTAGCATTCAGG
AAGAGATCAGAGGTCTGCTGGCTCCCTTATCATGTCCCTTATGGTGCTTCTGGCTCTGCA
GTTATTAGCATAGTGTTACCATCAACCACCTTAACTTCATTTTTCTTATTCAATACCTAG
GTAGGTAGATGCTAGATTCTGGAAATAAAATATGAGTCTCAAGTGGTCCTTGTCCTCTCT
CCCAGTCAAATTCTGAATCTAGTTGGCAAGATTCTGAAATCAAGGCATATAATCAGTAAT
AAGTGATGATAGAAGGGTATATAGAAGAATTTTATTATATGAGAGGGTGAAACCTAAAAT
GAAATGAAATCAGACCCTTGTCTTACACCATAAACAAAAATAAATTTGAATGGGTTAAAG

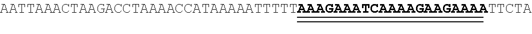

ATATTCATGTTGCAGCCGTTTTTTGAATTTGATATGAGAAGCAAAGGCAACAAAAGGAAA
AATAAAGAAGTGAGGCTACATCAAACTAAAAAATTTCCACACAAAAAAGAAAACAATGAA
CAAATGAAAGGTGAACCATGAAATGGCATATTTGCAAACCAAATATTTCTTAAATATTTT

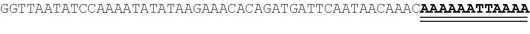

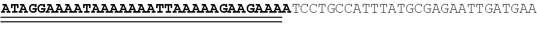

CCTGGAGGATGTAAAACTAAGAAAAATAAGCCTGACACAAAAAGACAAATACTACACAAC
CTTGCTCATATGTGAAACATAAAAAAGTCACTCTCATGGAAACAGACAGTAGAGGTATGG
TTTCCAGGGGTTGGGGGTGGGAGAATCAGGAAACTATTACTCAAAGGGTATAAAATTTCA
GTTATGTGGGATGAATAAATTCTAGATATCTAATGTACAGCATCGTGACTGTAGTTAATT
GTACTGTAAGTATATTTAAAATTTGCAAAGAGAGTAGATTTTTTTGTTTTTTTAGATGGA
GTTTTGCTCTTGTTGTCCAGGCTGGAGTGCAATGGCAAGATCTTGGCTCACTGCAACCTC
CGCCTCCTGGGTTCAAGCAAATCTCCTGCCTCAGCCTCCCGAGTAGOTGGGATTACAGGC
ATGCGACACCATGCCCAGCTAATTTTGTATTTTTAGTAGAGACGGGGTTTCTCCATGTTG
GTCAGGCTGATCCGCCTCCTCGGCCACCAAAGGGCTGGGATTACAGGCGTGACCACCGGG
CCTGGCCGAGAGTAGATCTTAAAAGCATTTACCACAAGAAAAAGGTAACTATGTGAGATA
ATGGGTATGTTAATTAGCTTGATTGTGGTAATCATTTCACAAGGTATACATATATTAAAA
CATCATGTTGTACACCTTAAATATATACAATTTTTATTTGTGAATGATACCTCAATAAAG
TTGAAGAATAATAAAAAAGAATAGACATCACATGAATTAAAAAACTAAAAAATAAAAAAA
TGCATCTTGATGATTAGAATTGCATTCTTGATTTTTCAGATACAAATATCCATTTGACTG
(SEQ ID NO: 13 - full sequence; SEQ ID NO: 14 - sequence in
italics).

b. Exemplary Triplex Forming Sequences

i. Beta Thalassemia

Gene editing molecules can be designed based on the guidance provided herein and otherwise known in the art. Exemplary triplex forming molecule and donor sequences, are provided in, for example, WO 1996/04027 1, WO/2010/123983, and U.S. Pat. No. 8,658,608, and in the working Examples below, and can be altered to include one or more of the modifications disclosed herein.

Triplex forming molecules can include a sequence substantially complementary to the polypurine strand of the polypyrimidine:polypurine target motif. In some embodiments, the triplex forming molecules target a region corresponding to nucleotides 566-577, optionally 566-583 or more of SEQ ID NO:14; a region corresponding to nucleotides 807-813, optionally 807-824 or more of SEQ ID NO: 14; or a region corresponding to nucleotides 605-611, optionally 605-621 of SEQ ID NO:14. Therefore in some embodiments, the triplex-forming molecules can form a triple-stranded molecule with the sequence including GAAAGAAAGAGA (SEQ ID NO:15) or TGCCCTGAAAGAAAGAGA (SEQ ID NO:16) or GGAGAAA(SEQ ID NO:17) or AGAATGGTGCAAAGAGG (SEQ ID NO:18) or AAAAGGG (SEQ ID NO:19) or ACATGATTAGCAAAAGGG (SEQ ID NO:20).

Accordingly, in some embodiments, the triplex-forming molecule includes the nucleic acid sequence CTTTCTTTCTCT (SEQ ID NO:21), preferable includes the sequence CTTTCTTTCTCT (SEQ ID NO:21) linked to the sequence TCTCTTTCTTTC (SEQ ID NO:22), or more preferable includes the sequence CTTTCTTTCTCT (SEQ ID NO:21) linked to the sequence TCTCTTTCTTTCAGGGCA (SEQ ID NO:23).

In some embodiments, the triplex-forming molecule includes the nucleic acid sequence TTTCCC (SEQ ID NO:24), preferable includes the sequence TTTCCC (SEQ ID NO:24) linked to the sequence CCCTTTT (SEQ ID NO:25), or more preferable includes the sequence TTTCCC (SEQ ID NO:24) linked to the sequence CCCTTTTGCTAATCATGT (SEQ ID NO:26).

In some embodiments, the triplex-forming molecule includes the nucleic acid sequence TTTCTCC (SEQ ID NO:27), preferable includes the sequence TTTCTCC (SEQ ID NO:27) linked to the sequence CCTCTTT (SEQ ID NO:28), or more preferable includes the sequence TTTCTCC (SEQ ID NO:27) linked to the sequence CCTCTTTGCACCATTCT (SEQ ID NO:29).

In some preferred embodiments, the triplex forming nucleic acid is a peptide nucleic acid including the sequence JTTTJTTTJTJT (SEQ ID NO:30) linked to the sequence TCTCTTTCTTTC (SEQ ID NO:22) or TCTCTTTCTTTCAGGGCA (SEQ ID NO:23); or

a peptide nucleic acid including the sequence TTTTJJJ (SEQ ID NO:31) linked to the sequence CCCTTTT (SEQ ID NO:25) or CCCTTTTGCTAATCATGT (SEQ ID NO:26);

or a peptide nucleic acid including the sequence TTTJTJJ (SEQ ID NO:32) linked to the sequence CCTCTTT (SEQ ID NO:28) or CCTCTTTGCACCATTCT (SEQ ID NO:29),

optionally, but preferably wherein one or more of the PNA monomers is a γPNA.

In specific embodiments, the triplex forming molecule is a peptide nucleic acid including the sequence lys-lys-lys-JTTTJTTTJTJT-OOO-TCTCTTTCTTTCAGGGCA-lys-lys-lys (SEQ ID NO:33), or

lys-lys-lys-TTTTJJJ-OOO-CCCTTTTGCTAATCATGT-lys-lys-lys (SEQ ID NO:34), or

lys-lys-lys-TTTJTJJ-OOO-CCTCTTTGCACCATTCT-lys-lys-lys (SEQ ID NO:35);

optionally, but preferably wherein one or more of the PNA monomers is a γPNA. In even more specific embodiments, the bolded and underlined residues are miniPEG-containing γPNA.

In other embodiments, the triplex forming nucleic acid is a peptide nucleic acid including the sequence TJTTTTJTTJ (SEQ ID NO:36) linked to the sequence CTTCTTTTCT (SEQ ID NO:37); or

TTJTTJTTTJ (SEQ ID NO:38) linked to the sequence CTTTCTTCTT (SEQ ID NO:39); or

JJJTJJTTJT (SEQ ID NO:40) linked to the sequence TCTTCCTCCC (SEQ ID NO:41); or

optionally, but preferably wherein one or more of the PNA monomers is a γPNA.

In specific embodiments, the triplex forming nucleic acid is a peptide nucleic acid including the sequence lys-lys-lys-TJTTTTJTTJ-OOO-CTTCTTTTCT-lys-lys-lys (SEQ ID NO:42) (IVS2-24); or

lys-lys-lys-TTJTTJTTTJ-OOO-CTTTCTTCTT-lys-lys-lys (SEQ ID NO:43) (IVS2-512); or

lys-lys-lys-JJJTJJTTJT-OOO-TCTTCCTCCC-lys-lys-lys (SEQ ID NO:44) (IVS2-830);

optionally, but preferably wherein one or more of the PNA monomers is a γPNA. In even more specific embodiments, the bolded and underlined residues are miniPEG-containing γPNA.

ii. Sickle Cell Disease

Preferred sequences that target the sickle cell disease mutation (20) in the beta globin gene are also provided. In some embodiments, the triplex-forming molecule includes the nucleic acid sequence CCTCTTC (SEQ ID NO:45), preferable includes the sequence CCTCTTC (SEQ ID NO:45) linked to the sequence CTTCTCC (SEQ ID NO:46), or more preferable includes the sequence CCTCTTC (SEQ ID NO:45) linked to the sequence CTTCTCCAAAGGAGT (SEQ ID NO:47) or CTTCTCCACAGGAGTCAG (SEQ ID NO:48) or CTTCTCCACAGGAGTCAGGTGC (SEQ ID NO: 158).

In some embodiments, the triplex-forming molecule includes the nucleic acid sequence TTCCTCT (SEQ ID NO:49), preferable includes the sequence TTCCTCT (SEQ ID NO:49) linked to the sequence TCTCCTT (SEQ ID NO:50), or more preferable includes the sequence TTCCTCT (SEQ ID NO:49) linked to the sequence TCTCCTTAAACCTGT (SEQ ID NO:51) or TCTCCTTAAACCTGTCTT (SEQ ID NO:172).

In some embodiments, the triplex-forming molecule includes the nucleic acid sequence TCTCTTCT (SEQ ID NO:52), preferable includes the sequence TCTCTTCT (SEQ ID NO:52) linked to the sequence TCTTCTCT (SEQ ID NO:53), or more preferable includes the sequence TCTCTTCT (SEQ ID NO:52) linked to the sequence TCTTCTCTGTCTCCAC (SEQ ID NO:54) or TCTTCTCTGTCTCCACAT (SEQ ID NO:55).

In some preferred embodiments for correction of Sickle Cell Disease Mutation, the triplex forming nucleic acid is a peptide nucleic acid including the sequence JJTJTTJ (SEQ ID NO:56) linked to the sequence CTTCTCC (SEQ ID NO:46) or CTTCTCCAAAGGAGT (SEQ ID NO:47) or CTTCTCCACAGGAGTCAG (SEQ ID NO:48) or CTTCTCCACAGGAGTCAGGTGC (SEQ ID NO: 158);

or a peptide nucleic acid including the sequence TTJJTJT (SEQ ID NO:175) linked to the sequence TCTCCTT (SEQ ID NO:50) or TCTCCTTAAACCTGT (SEQ ID NO:51) or TCTCCTTAAACCTGTCTT (SEQ ID NO:172);

or a peptide nucleic acid including the sequence TJTJTTJT (SEQ ID NO:176) linked to the sequence TCTTCTCT (SEQ ID NO:53) or TCTTCTCTGTCTCCAC (SEQ ID NO:54) or TCTTCTCTGTCTCCACAT (SEQ ID NO:55);

optionally, but preferably wherein one or more of the PNA monomers is a γPNA.

In specific embodiments for correction of Sickle Cell Disease Mutation, the triplex forming nucleic acid is a peptide nucleic acid including the sequence lys-lys-lys-JJTJTTJ-OOO-CTTCTCCAAAGGAGT-lys-lys-lys (SEQ ID NO:160); or

lys-lys-lys-TTJJTJT-OOO-TCTCCTTAAACCTGT-lys-lys-lys (SEQ ID NO:57); or

lys-lys-lys-TTJJTJT-OOO-TCTCCTTAAACCTGTCTT-lys-lys-lys (SEQ ID NO:174), or

lys-lys-lys-TJTJTTJT-OOO-TCTTCTCTGTCTCCAC-lys-lys-lys (SEQ ID NO:58) (tc816); or

lys-lys-lys-JJTJTTJ-OOO-CTTCTCCACAGGAGTCAG-lys-lys-lys (SEQ ID NO:59); or

lys-lys-lys-JJTJTTJ-OOO-CTTCTCCACAGGAGTCAG-lys-lys-lys (SEQ ID NO:59) (SCD-tcPNA 1A); or

lys-lys-lys-JJTJTTJ-OOO-CTTCTCCACAGGAGTCAG-lys-lys-lys (SEQ ID NO:59) (SCD-tcPNA 1B); or

lys-lys-lys-JJTJTTJ-OOO-CTTCTCCACAGGAGTCAG-lys-lys-lys (SEQ ID NO:59) (SCD-tcPNA IC); or

lys-lys-lys-JJTJTTJ-OOO-CTTCTCCACAGGAGTCAGGTGC-lys-lys-lys (SEQ ID NO:161) (SCD-tcPNA 1D); or

lys-lys-lys-JJTJTTJ-OOO-CTTCTCCACAGGAGTCAGGTGC-lys-lys-lys (SEQ ID NO:161) (SCD-tcPNA 1E); or

lys-lys-lys-JJTJTTJ-OOO-CTTCTCCACAGGAGTCAGGTGC-lys-lys-lys (SEQ ID NO:161) (SCD-tcPNA 1F); or

lys-lys-lys-TJTJTTJT-OOO-TCTTCTCTGTCTCCACAT-lys-lys-lys (SEQ ID NO:60);

optionally, but preferably wherein one or more of the PNA monomers is a γPNA. In even more specific embodiments, the bolded and underlined residues are miniPEG-containing γPNA.

c. Exemplary Donors

In some embodiments, the triplex forming molecules are used in combination with a donor oligonucleotide for correction of IVS2-654 mutation that includes the sequence 5′AAAGAATAACAGTGATAATTTCTGGGTTAAGGCAATAGCAATA TCTCTGCATATAAATAT 3′ (SEQ ID NO:65) with the correcting IVS2-654 nucleotide underlined, or a functional fragment thereof that is suitable and sufficient to correct the IVS2-654 mutation.

Other exemplary donor sequences include, but are not limited to, DonorGFP-IVS2-1 (Sense) 5′-GTTCAGCGTGTCCGGCGAGGGCGAGGTGAGTCTATGGGACCC TTGATGTTT-3′ (SEQ ID NO:61), DonorGFP-IVS2-1 (Antisense) 5′-AAACATCAAGGGTCCCATAGACTCACCTCGCCCTCGCCGGAC ACGCTGAAC-3′ (SEQ ID NO:62), and, or a functional fragment thereof that is suitable and sufficient to correct a mutation.

In some embodiments, a Sickle Cells Disease mutation can be corrected using a donor having the sequence

5′CTTGCCCCACAGGGCAGTAACGGCAGATTTTTC [symbol] CGG CGTTAAATGCACCATGGTGTCTGTTTGAGGT 3′ (SEQ ID NO:63), or a functional fragment thereof that is suitable and sufficient to correct a mutation, wherein the three boxed nucleotides represent the corrected codon 6 which reverts the mutant Valine (associated with human sickle cell disease) back to the wildtype Glutamic acid and nucleotides in bold font (without underlining) represent changes to the genomic DNA but not to the encoded amino acid; or

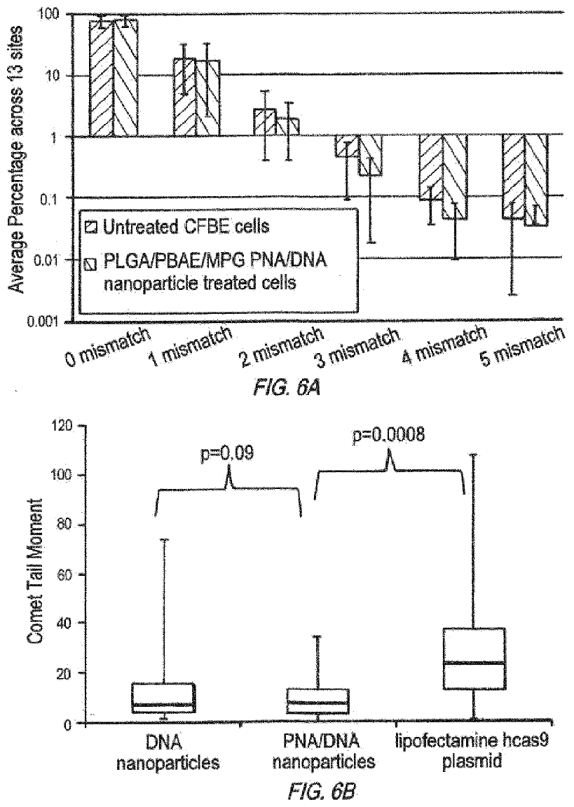

5′ACAGACACCATGGTGCACCTGACTCCTGAGGAGAAGTCT GCCGTTACTGCC 3′ (SEQ ID NO:64), or a functional fragment thereof that is suitable and sufficient to correct a mutation, wherein the bolded and underlined residue the correction (see, e.g., FIG. 6).

5′T(s)T(s)G(s)CCCCACAGGGCAGTAACGGCAGACTTCTCCTC AGGAGTCAGGTGCACCATGGTGTCTGTT(s)T(s)G(s)3′ (SEQ ID NO: 173), or a functional fragment thereof that is suitable and sufficient to correct a mutation, wherein the bolded and underlined residue is the correction and “(s)” indicates an optional phosphorothioate internucleoside linkage.

2. Cystic Fibrosis

The disclosed compositions and methods can be used to treat cystic fibrosis. Cystic fibrosis (CF) is a lethal autosomal recessive disease caused by defects in the cystic fibrosis transmembrane conductance regulator (CFTR), an ion channel that mediates Cl-transport. Lack of CFTR function results in chronic obstructive lung disease and premature death due to respiratory failure, intestinal obstruction syndromes, exocrine and endocrine pancreatic dysfunction, and infertility (Davis, et al., Pediatr Rev., 22(8):257-64 (2001)). The most common mutation in CF is a three base-pair deletion (F508del) resulting in the loss of a phenylalanine residue, causing intracellular degradation of the CFTR protein and lack of cell surface expression (Davis, et al., Am J Respir Crit Care Med., 173(5):475-82 (2006)). In addition to this common mutation there are many other mutations that occur and lead to disease including a class of mutations due to premature stop codons, nonsense mutations. In fact nonsense mutations account for approximately 10% of disease causing mutations. Of the nonsense mutations G542X and W1282X are the most common with frequencies of 2.6% and 1.6% respectfully.

Although CF is one of the most rigorously characterized genetic diseases, current treatment of patients with CF focuses on symptomatic management rather than primary correction of the genetic defect. Gene therapy has remained an elusive target in CF, because of challenges of in vivo delivery to the lung and other organ systems (Armstrong, et al., Archives of disease in childhood (2014) doi: 10.1136/archdischild-2012-302158. PubMed PMID: 24464978). In recent years, there have been many advances in gene therapy for treatment of diseases involving the hematolymphoid system, where harvest and ex vivo manipulation of cells for autologous transplantation is possible: some examples include the use of zinc finger nucleases targeting CCR5 to produce HIV-1 resistant cells (Holt, et al., Nature biotechnology, 28(8):839-47 (2010)) correction of the ABCD1 gene by lentiviral vectors for treatment of adrenoleukodystrophy (Cartier, et al., Science, 326(5954):818-23 (2009)) and correction of SCID due to ADA deficiency using retroviral gene transfer (Aiuti, et al., The New England Journal Of Medicine, 360(5):447-58 (2009).

Unfortunately, harvest and autologous transplant is not an option in CF, due to the involvement of the lung and other internal organs. As one approach, the UK Cystic Fibrosis Gene Therapy Consortium has tested liposomes to deliver plasmids containing cDNA encoding CFTR to the lung (Alton, et al., Thorax, 68(11):1075-7 (2013)), Alton, et al., The Lancet Respiratory Medicine, (2015). doi: 10.1016/S2213-2600(15)00245-3. PubMed PMID: 26149841.) other clinical trials have used viral vectors for delivery of the CFTR gene or CFTR expression plasmids that are compacted by polyethylene glycol-substituted lysine 30-mer peptides with limited success (Konstan, et al., Human Gene Therapy, 15(12):1255-69 (2004)). Moreover, delivery of plasmid DNA for gene addition without targeted insertion does not result in correction of the endogenous gene and is not subject to normal CFTR gene regulation, and virus-mediated integration of the CFTR cDNA could introduce the risk of non-specific integration into important genomic sites.

However, it has been discovered that triplex-forming PNA molecules and donor DNA can be used to correct mutations leading to cystic fibrosis. In preferred embodiments, the compositions are administered by intranasal or pulmonary delivery. The compositions can be administered in an effective amount to induce or enhance gene correction in an amount effective to reduce one or more symptoms of cystic fibrosis. For example, in some embodiments, the gene correction occurs at an amount effective to improve impaired response to cyclic AMP stimulation, improve hyperpolarization in response to forskolin, reduction in the large lumen negative nasal potential, reduction in inflammatory cells in the bronchioalveolar lavage (BAL), improve lung histology, or a combination thereof. In some embodiments, the target cells are cells, particularly epithelial cells, that make up the sweat glands in the skin, that line passageways inside the lungs, liver, pancreas, or digestive or reproductive systems. In particular embodiments, the target cells are bronchial epithelial cells. While permanent genomic change using PNA/DNA is less transient than plasmid-based approaches and the changes will be passed on to daughter cells, some modified cells may be lost over time with regular turnover of the respiratory epithelium. In some embodiments, the target cells are lung epithelial progenitor cells. Modification of lung epithelial progenitors can induce more long-term correction of phenotype.

Sequences for the human cystic fibrosis transmembrane conductance regulator (CFTR) are known in the art, see, for example, GenBank Accession number: AH006034.1, and compositions and methods of targeted correction of CFTR are described in McNeer, et al., Nature Communications, 6:6952, (DOI 10.1038/ncomms7952), 11 pages.

a. Exemplary F508del Target Sites

In some embodiments, the triplex-forming molecules are designed to target the CFTR gene at nucleotides 9,152-9,159 (TTTCCTCT (SEQ ID NO:70)) or 9,159-9,168 (TTTCCTCTATGGGTAAG (SEQ ID NO:71) of accession number AH006034.1, or the non-coding strand (e.g., 3′-5′ complementary sequence) corresponding to nucleotides 9,152-9,159 or 9,152-9,168 (e.g., 5′-AGAGGAAA-3′ (SEQ ID NO:72), or 5′-CTTACCCATAGAGGAAA-3′ (SEQ ID NO:73)).

In some embodiments, the triplex-forming molecules are designed to target the CFTR gene at nucleotides 9,039-9,046 (5′-AGAAGAGG-3′ (SEQ ID NO:74), or 9,030-9,046 (5′-ATGCCAACTAGAAGAGG-3′ (SEQ ID NO:75)) of accession number AH006034.1, or the non-coding strand (e.g., 3′-5′ complementary sequence) corresponding to nucleotides (5′ CCTCTTCT 3′ (SEQ ID NO:76)) or (5′ CCTCTTCTAGTTGGCAT 3′ (SEQ ID NO:77).

In some embodiments, the triplex-forming molecules are designed to target the CFTR gene at nucleotides 8,665-8,683 (CTTTCCCTT (SEQ ID NO:78)) or 8,665-8,682 (CTTTCCCTTGTATCTTTT (SEQ ID NO:79) of accession number AH006034.1, or the non-coding strand (e.g., 3′-5′ complementary sequence) corresponding to nucleotides 8,665-8,683 or 8,665-8,682 (e.g., 5′-AAGGGAAAG-3′ (SEQ ID NO:80), or 5′-AAAAGATAC AAGGGAAAG-3′ (SEQ ID NO:81)).

In some embodiments, the triplex-forming molecules are designed to target the W1282X mutation in CFTR gene at the sequence GAAGGAGAAA (SEQ ID NO:163), AAAAGGAA (SEQ ID NO:164), or AGAAAAAAGG (SEQ ID NO: 165), or the inverse complement thereof. See FIG. 8C.

In some embodiments, the triplex-forming molecules are designed to target the G542X mutation in CFTR gene at the sequence AGAAAAA (SEQ ID NO: 166), AGAGAAAGA (SEQ ID NO:167), or AAAGAAA (SEQ ID NO:168), or the inverse complement thereof. See FIG. 9C.

b. Exemplary Triplex Forming Sequences and Donors

i. F508del

In some embodiments, the triplex-forming molecule includes the nucleic acid sequence includes TCTCCTTT (SEQ ID NO:82), preferably linked to the sequence TTTCCTCT (SEQ ID NO:83) or more preferably includes TCTCCTTT (SEQ ID NO:82) linked to the sequence TTTCCTCTATGGGTAAG (SEQ ID NO:84); or

includes TCTTCTCC (SEQ ID NO:85) preferably linked to the sequence CCTCTTCT (SEQ ID NO:86), or more preferably includes TCTTCTCC (SEQ ID NO:85) linked to CCTCTTCTAGTTGGCAT (SEQ ID NO:87); or

includes TTCCCTTTC (SEQ ID NO:88), preferable includes the sequence TTCCCTTTC (SEQ ID NO:88) linked to the sequence CTTTCCCTT (SEQ ID NO:89), or more preferable includes the sequence TTCCCTTTC (SEQ ID NO:88) linked to the sequence CTTTCCCTTGTATCTTTT (SEQ ID NO:90).

In some preferred embodiments, the triplex forming nucleic acid is a peptide nucleic acid including the sequence TJTJJTTT (SEQ ID NO:91), linked to the sequence TTTCCTCT (SEQ ID NO:83) or TTTCCTCTATGGGTAAG (SEQ ID NO:84); or

TJTTJTJJ (SEQ ID NO:177) linked to the sequence CCTCTTCT (SEQ ID NO:86), or CCTCTTCTAGTTGGCAT (SEQ ID NO:87);

or TTJJJTTTJ (SEQ ID NO:92) linked to the sequence CTTTCCCTT (SEQ ID NO:89), or CTTTCCCTTGTATCTTTT (SEQ ID NO:90);

optionally, but preferably wherein one or more of the PNA monomers is a γPNA.

In specific embodiments the triplex forming nucleic acid is a peptide nucleic acid including the sequence is lys-lys-lys-TJTJJTTT-OOO-TTTCCTCTATGGGTAAG-lys-lys-lys (SEQ ID NO:93) (hCFPNA2); or

lys-lys-lys-TJTJJTTT-OOO-TTTCCTCTATGGGTAAG-lys-lys-lys (SEQ ID NO:93); or

lys-lys-lys-TJTTJTJJ-OOO-CCTCTTCTAGTTGGCAT-lys-lys-lys (SEQ ID NO:94) (hCFPNA1); or

lys-lys-lys-TTJJJTTTJ-OOO-CTTTCCCTTGTATCTTTT-lys-lys-lys (SEQ ID NO:95) (hCFPNA3);

optionally, but preferably wherein one or more of the PNA monomers is a γPNA. In even more specific embodiments, the bolded and underlined residues are miniPEG-containing γPNA.

In some embodiments, a donor that can be used for CFTR gene correction, particularly in combination with the foregoing triplex forming molecules, includes the sequence 5′TTCTGTATCTATATTCATCATAGGAAACACCAAAGATAATGTTCT CCTTAATGGTGCCAGG3′ (SEQ ID NO:96), or a functional fragment thereof that is suitable and sufficient to correct the F508del mutation in the cystic fibrosis transmembrane conductance regulator (CFTR) gene.

ii. W1282 Mutation Site

In some embodiments, the triplex-forming molecule includes the nucleic acid sequence CTTCCTCTTT (SEQ ID NO:97), preferable includes the sequence CTTCCTCTTT (SEQ ID NO:97) linked to the sequence TTTCTCCTTC (SEQ ID NO:98), or more preferable includes the sequence CTTCCTCTTT (SEQ ID NO:97) linked to the sequence TTTCTCCTTCAGTGTTCA (SEQ ID NO:99); or

the triplex-forming molecule includes the nucleic acid sequence TTTTCCT (SEQ ID NO: 100), preferable includes the sequence TTTTCCT (SEQ ID NO:100) linked to the sequence TCCTTTT (SEQ ID NO:101), or more preferable includes the sequence TTTTCCT (SEQ ID NO: 100) linked to the sequence TCCTTTTGCTCACCTGTGGT (SEQ ID NO: 102); or

the triplex-forming molecule includes the nucleic acid sequence TCTTTTTTCC (SEQ ID NO: 103), preferable includes the sequence TCTTTTTTCC (SEQ ID NO: 103) linked to the sequence CCTTTTTTCT (SEQ ID NO: 104), or more preferable includes the sequence TCTTTTTTCC (SEQ ID NO: 103) linked to the sequence CCTTTTTTCTGGCTAAGT (SEQ ID NO: 105).

In preferred embodiments, the triple forming nucleic acid is a peptide nucleic acid including the sequence JTTJJTJTTT (SEQ ID NO: 106) linked to the sequence TTTCTCCTTC (SEQ ID NO:98) or TTTCTCCTTCAGTGTTCA (SEQ ID NO:99); or

a peptide nucleic acid including the sequence TTTTJJT (SEQ ID NO: 107) linked to the sequence TCCTTTT (SEQ ID NO:101) or linked to the sequence TCCTTTTGCTCACCTGTGGT (SEQ ID NO: 102); or

a peptide nucleic acid including the sequence TJTTTTTTJJ (SEQ ID NO: 108) linked to the sequence CCTTTTTTCT (SEQ ID NO: 104) or linked to the sequence CCTTTTTTCTGGCTAAGT (SEQ ID NO: 105);

optionally, but preferably wherein one or more of the PNA monomers is a γPNA.

In specific embodiments, the triplex forming nucleic acid is a peptide nucleic acid including the sequence lys-lys-lys-JTTJJTJTTT-OOO-TTTCTCCTTCAGTGTTCA-lys-lys-lys (SEQ ID NO: 155) (tcPNA-1236); or

lys-lys-lys-TTTTJJT-OOO-TCCTTTTGCTCACCTGTGGT-lys-lys-lys (SEQ ID NO:156) (tcPNA-1314); or

lys-lys-lys-TJTTTTTTJJ-OOO-CCTTTTTTCTGGCTAAGT-lys-lys-lys (SEQ ID NO:157) (tcPNA-1329);

optionally, but preferably wherein one or more of the PNA monomers is a γPNA. In even more specific embodiments, the bolded and underlined residues are miniPEG-containing γPNA.

In some embodiments, a donor that can be used for CFTR gene correction, particularly in combination with the foregoing triplex forming molecules, includes the sequence T(s)C(s)T(s)-TGGGATTCAATAACCTTGCAGACAGTGGAGGAAGGCCTTTGGCG TGATACCACAGG-(s)T(s)G(s) (SEQ ID NO:109) or a functional fragment thereof that is suitable and sufficient to correct a mutation in CFTR, wherein the bolded and underlined nucleotides are inserted mutations for gene correction, and “(s)” indicates an optional phosphorothioate internucleoside linkage. See also, FIGS. 8A-8C, W1282X.

iii. G542X Mutation Site

In some embodiments, the triplex-forming molecule includes the nucleic acid sequence TCTTTTT (SEQ ID NO:110), preferable includes the sequence TCTTTTT (SEQ ID NO: 110) linked to the sequence TTTTTCT (SEQ ID NO:111), or more preferable includes the sequence TCTTTTT (SEQ ID NO:110) linked to the sequence TTTTTCTGTAATTTTTAA (SEQ ID NO:112); or

the triplex-forming molecule includes the nucleic acid sequence TCTCTTTCT (SEQ ID NO: 113), preferable includes the sequence TCTCTTTCT (SEQ ID NO: 113) linked to the sequence TCTTTCTCT (SEQ ID NO: 114), or more preferable includes the sequence TCTCTTTCT (SEQ ID NO: 113) linked to the sequence TCTTTCTCTGCAAACTT (SEQ ID NO:115); or

the triplex-forming molecule includes the nucleic acid sequence TTTCTTT (SEQ ID NO:116), preferable includes the sequence TTTCTTT (SEQ ID NO:116) linked to the sequence TTTCTTT (SEQ ID NO: 116), or more preferable includes the sequence TTTCTTT (SEQ ID NO: 116) linked to the sequence TTTCTTTAAGAACGAGCA (SEQ ID NO: 117).

In preferred embodiments, the triple forming nucleic acid is a peptide nucleic acid including the sequence TJTTTTT (SEQ ID NO: 118) linked to the sequence TTTTTCT (SEQ ID NO:111) or TTTTTCTGTAATTTTTAA (SEQ ID NO:112); or

a peptide nucleic acid including the sequence TJTJTTTJT (SEQ ID NO: 119) linked to the sequence TCTTTCTCT (SEQ ID NO: 114) or linked to the sequence TCTTTCTCTGCAAACTT (SEQ ID NO:115); or

a peptide nucleic acid including the sequence TTTJTTT (SEQ ID NO: 120) linked to the sequence TTTCTTT (SEQ ID NO: 116) or linked to the sequence TTTCTTTAAGAACGAGCA (SEQ ID NO:117);

optionally, but preferably wherein one or more of the PNA monomers is a γPNA.

In specific embodiments, the triplex forming nucleic acid is a peptide nucleic acid including the sequence lys-lys-lys-TJTTTTT-OOO-TTTTTCTGTAATTTTTAA-lys-lys-lys (SEQ ID NO:121) (tcPNA-302); or

lys-lys-lys-TJTJTTTJT-OOO-TCTTTCTCTGCAAACTT-lys-lys-lys (SEQ ID NO: 122) (tcPNA-529); or

lys-lys-lys-TTTJTTT-OOO-TTTCTTTAAGAACGAGCA-lys-lys-lys (SEQ ID NO:123) (tcPNA-586);

optionally, but preferably wherein one or more of the PNA monomers is a γPNA. In even more specific embodiments, the bolded and underlined residues are miniPEG-containing γPNA.

In some embodiments, a donor that can be used for CFTR gene correction, particularly in combination with the foregoing triplex forming molecules, includes the sequence T(s)C(s)C(s)-AAGTTTGCAGAGAAAGATAATATAGTCCTTGGAGAAGGAGGAAT CACCCTGAGTGGA-G(s)G(s)T(s) (SEQ ID NO: 124), or a functional fragment thereof that is suitable and sufficient to correct a mutation in CFTR, wherein the bolded and underlined nucleotides are inserted mutations for gene correction, and “(s)” indicates an optional phosphorothioate internucleoside linkage. See also, FIGS. 9A-9C, G542X.

3. HIV

The gene editing compositions can be used to treat infections, for example those caused by HIV.

a. Exemplary Target Sites

The target sequence for the triplex-forming molecules is within or adjacent to a human gene that encodes a cell surface receptor for human immunodeficiency virus (HIV). Preferably, the target sequence of the triplex-forming molecules is within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences that regulate expression of the target gene, including promoter or enhancer sequences.

The target sequence can be within or adjacent to any gene encoding a cell surface receptor that facilitates entry of HIV into cells. The molecular mechanism of HIV entry into cells involves specific interactions between the viral envelope glycoproteins (env) and two target cell proteins, CD4 and the chemokine receptors. HIV cell tropism is determined by the specificity of the env for a particular chemokine receptor, a 7 transmembrane-spanning, G protein-coupled receptor (Steinberger, et al., Proc. Natl. Acad. Sci. USA. 97: 805-10 (2000)). The two major families of chemokine receptors are the CXC chemokine receptors and the CC chemokine receptors (CCR) so named for their binding of CXC and CC chemokines, respectively. While CXC chemokine receptors traditionally have been associated with acute inflammatory responses, the CCRs are mostly expressed on cell types found in connection with chronic inflammation and T-cell-mediated inflammatory reactions: eosinophils, basophils, monocytes, macrophages, dendritic cells, and T cells (Nansen, et al. 2002, Blood 99:4). In one embodiment, the target sequence is within or adjacent to the human genes encoding chemokine receptors, including, but not limited to, CXCR4, CCR5, CCR2b, CCR3, and CCR1.

In a preferred embodiment, the target sequence is within or adjacent to the human CCR5 gene. The CCR5 chemokine receptor is the major co-receptor for R5-tropic HIV strains, which are responsible for most cases of initial, acute HIV infection. Individuals who possess a homozygous inactivating mutation, referred to as the Δ32 mutation, in the CCR5 gene are almost completely resistant to infection by R5-tropic HIV-1 strains. The Δ32 mutation produces a 32 base pair deletion in the CCR5 coding region.

Another naturally occurring mutation in the CCR5 gene is the m303 mutation, characterized by an open reading frame single T to A base pair transversion at nucleotide 303 which indicates a cysteine to stop codon change in the first extracellular loop of the chemokine receptor protein at amino acid 101 (C101X) (Carrington et al. 1997). Mutagenesis assays have not detected the expression of the m303 co-receptor on the surface of CCR5 null transfected cells which were found to be non-susceptible to HIV-1 R5-isolates in infection assays (Blanpain, et al. (2000).

Compositions and methods for targeted gene therapy using triplex-forming oligonucleotides and peptide nucleic acids for treating infectious diseases such as HIV are described in U.S. Application No. 2008/050920 and WO 2011/133803. Each provides sequences of triplex forming molecules, target sequences, and donor oligonucleotides that can be utilized in the compositions and methods provided herein.

For example, individuals having the homozygous Δ32 inactivating mutation in the CCR5 gene display no significant adverse phenotypes, suggesting that this gene is largely dispensable for normal human health. This makes the CCR5 gene a particularly attractive target for targeted mutagenesis using the triplex-forming molecules disclosed herein. The gene for human CCR5 is known in the art and is provided at GENBANK accession number NM_000579. The coding region of the human CCR5 gene is provided by nucleotides 358 to 1416 of GENBANK accession number NM_000579.

In some embodiments, the target region is a polypurine site within or adjacent to a gene encoding a chemokine receptor including CXCR4, CCR5, CCR2b, CCR3, and CCR1. In a preferred embodiment, the target region is a polypurine or homopurine site within the coding region of the human CCR5 gene. Three homopurine sites in the coding region of the CCR5 gene that are especially useful as target sites for triplex-forming molecules are from positions 509-518, 679-690 and 900-908 relative to the ATG start codon. The homopurine site from 679-690 partially encompasses the site of the nonsense mutation created by the Δ32 mutation. Triplex-forming molecules that bind to this target site are particularly useful.

b. Exemplary Triplex Forming Sequences

In some embodiments, the triplex-forming molecule includes the nucleic acid sequence CTCTTCTTCT (SEQ ID NO: 125), preferable includes the sequence CTCTTCTTCT (SEQ ID NO: 125) linked to the sequence TCTTCTTCTC (SEQ ID NO: 126), or more preferable includes the sequence CTCTTCTTCT (SEQ ID NO: 125) linked to the sequence TCTTCTTCTCATTTC (SEQ ID NO: 127).

In some embodiments, the triplex-forming molecule includes the nucleic acid sequence CTTCT (SEQ ID NO:128), preferable includes the sequence CTTCT (SEQ ID NO: 128) linked to the sequence TCTTC (SEQ ID NO: 129) or TCTTCTTCTC (SEQ ID NO: 130), or more preferable includes the sequence CTTCT (SEQ ID NO:128) linked to the sequence TCTTCTTCTCATTTC (SEQ ID NO:131).

In preferred embodiments, the triplex forming nucleic acid is a peptide nucleic acid including the sequence JTJTTJTTJT (SEQ ID NO:132) linked to the sequence TCTTCTTCTC (SEQ ID NO: 126) or TCTTCTTCTCATTTC (SEQ ID NO:127);

or JTTJT (SEQ ID NO:133) linked to the sequence TCTTC (SEQ ID NO: 129) or TCTTCTTCTC (SEQ ID NO: 130) or more preferably TCTTCTTCTCATTTC (SEQ ID NO:131);

optionally, but preferably wherein one or more of the PNA monomers is a γPNA.

In specific embodiments, the triplex forming nucleic acid is a peptide nucleic acid including the sequence Lys-Lys-Lys-JTJTTJTTJT-OOO-TCTTCTTCTCATTTC-Lys-Lys-Lys (SEQ ID NO:134) (PNA-679);

or Lys-Lys-Lys-JTTJT-OOO-TCTTCTTCTCATTTC-Lys-Lys-Lys (SEQ ID NO:135) (tcPNA-684) optionally, but preferably wherein one or more of the PNA monomers is a γPNA. In even more specific embodiments, the bolded and underlined residues are miniPEG-containing γPNA.

c. Exemplary Donor Sequences

In some embodiments, the triplex forming molecules are used in combination with one or more donor oligonucleotides such as donor 591 having the sequence: 5′ AT TCC CGA GTA GCA GAT GAC CAT GAC AGC TTA GGG CAG GAC CAG CCC CAA GAT GAC TAT C 3′ (SEQ ID NO: 136), or donor 597 having the sequence 5′ TT TAG GAT TCC CGA GTA GCA GAT GAC CCC TCA GAG CAG CGG CAG GAC CAG CCC CAA GAT G 3′ (SEQ ID NO:137), which can be used in combination to induce two different non-sense mutations, one in each allele of the CCR5 gene, in the vicinity of the Δ32 deletion (mutation sites are bolded); or a functional fragment thereof that is suitable and sufficient to introduce a non-sense mutation in at least one allele of the CCR5 gene.

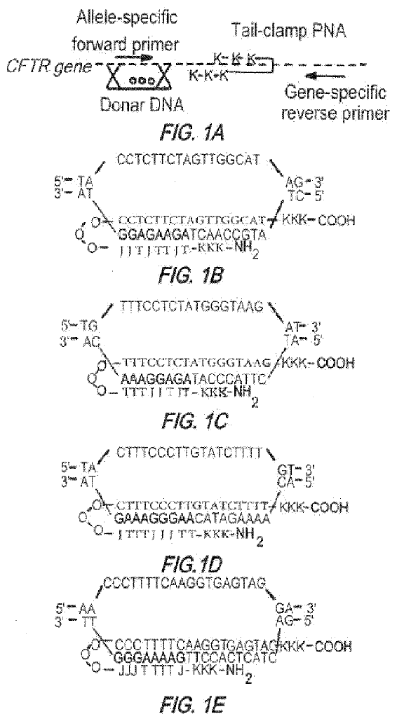

In another preferred embodiment, donor oligonucleotides are designed to span the Δ32 deletion site (see, e.g., FIG. 1 of WO 2011/133803) and induce changes into a wildtype CCR5 allele that mimic the Δ32 deletion. Donor sequences designed to target the Δ32 deletion site may be particularly usefully to facilitate knockout of the single wildtype CCR5 allele in heterozygous cells.

Preferred donor sequences designed to target the Δ32 deletion site include, but are not limited to,

Donor DELTA32JDC:
(SEQ ID NO: 138)
5′ GATGACTATCTTTAATGTCTGGAAATTCTTCCAGA
ATTAATTAAGACTGTATGGAAAATGAGAGC 3′;
Donor DELTAJDC2:
(SEQ ID NO: 139)
5′ CCCCAAGATGACTATCTTTAATGTCTGGAACGATC
ATCAGAATTGATACTGACTGTATGGAAAATG 3′;
and
Donor DELTA32RSB:
(SEQ ID NO: 140)
5′ GATGACTATCTTTAATGTCTGGAAATTCTACTAGA
ATTGATACTGACTGTATGGAAAATGAGAGC 3′,

or a functional fragment of SEQ ID NO:138, 139, or 140 that is suitable and sufficient to introduce mutation CCR5 gene.

4. Lysosomal Storage Diseases

The disclosed compositions and methods compositions can also be used to treat lysosomal storage diseases. Lysosomal storage diseases (LSDs) are a group of more than 50 clinically-recognized, rare inherited metabolic disorders that result from defects in lysosomal function (Walkley, J. Inherit. Metab. Dis., 32(2):181-9 (2009)). Lysosomal storage disorders are caused by dysfunction of the cell's lysosome orangelle, which is part of the larger endosomal/lysosomal system. Together with the ubiquitin-proteosomal and autophagosomal systems, the lysosome is essential to substrate degradation and recycling, homeostatic control, and signaling within the cell. Lysosomal dysfunction is usually the result of a deficiency of a single enzyme necessary for the metabolism of lipids, glycoproteins (sugar containing proteins) or mucopolysaccharides (long unbranched polysaccharides consisting of a repeating disaccharide unit; also known as glycosaminoglycans, or GAGs) which are fated for breakdown or recycling. Enzyme deficiency reduces or prevents break down or recycling of the unwanted lipids, glycoproteins, and GAGs, and results in buildup or “storage” of these materials within the cell. Most lysosomal diseases show widespread tissue and organ involvement, with brain, viscera, bone and connective tissues often being affected. More than two-thirds of lysosomal diseases affect the brain. Neurons appear particularly vulnerable to lysosomal dysfunction, exhibiting a range of defects from specific axonal and dendritic abnormalities to neuron death.

Individually, LSDs occur with incidences of less than 1:100,000, however, as a group the incidence is as high as 1 in 1,500 to 7,000 live births (Staretz-Chacham, et al., Pediatrics, 123(4):1191-207 (2009)). LSDs are typically the result of inborn genetic errors. Most of these disorders are autosomal recessively inherited, however a few are X-linked recessively inherited, such as Fabry disease and Hunter syndrome (MPS II). Affected individuals generally appear normal at birth, however the diseases are progressive. Develop of clinical disease may not occur until years or decades later, but is typically fatal. Lysosomal storage diseases affect mostly children and they often die at a young and unpredictable age, many within a few months or years of birth. Many other children die of this disease following years of suffering from various symptoms of their particular disorder. Clinical disease may be manifest as mental retardation and/or dementia, sensory loss including blindness or deafness, motor system dysfunction, seizures, sleep and behavioral disturbances, and so forth. Some people with Lysosomal storage disease have enlarged livers (hepatomegaly) and enlarged spleens (splenomegaly), pulmonary and cardiac problems, and bones that grow abnormally.

Treatment for many LSDs is enzyme replacement therapy (ERT) and/or substrate reduction therapy (SRT), as wells as treatment or management of symptoms. The average annual cost of ERT in the United States ranges from $90,000 to $565,000. While ERT has significant systemic clinical efficacy for a variety of LSDs, little or no effects are seen on central nervous system (CNS) disease symptoms, because the recombinant proteins cannot penetrate the blood-brain barrier. Allogeneic hematopoietic stem cell transplantation (HSCT) represents a highly effective treatment for selected LSDs. It is currently the only means to prevent the progression of associated neurologic sequelae. However, HSCT is expensive, requires an HLA-matched donor and is associated with significant morbidity and mortality. Recent gene therapy studies suggest that LSDs are good targets for this type of treatment.

Compositions and methods for targeted gene therapy using triplex-forming oligonucleotides and peptide nucleic acids for treating lysosomal storage diseases are described in WO 2011/133802, which provides sequences of triplex forming molecules, target sequences, and donor oligonucleotides that can be utilized in the compositions and methods provided herein.

For example, the disclosed compositions and methods can be are employed to treat Gaucher's disease (GD). Gaucher's disease, also known as Gaucher syndrome, is the most common lysosomal storage disease. Gaucher's disease is an inherited genetic disease in which lipid accumulates in cells and certain organs due to deficiency of the enzyme glucocerebrosidase (also known as acid β-glucosidase) in lysosomes. Glucocerebrosidase enzyme contributes to the degradation of the fatty substance glucocerebroside (also known as glucosylceramide) by cleaving b-glycoside into b-glucose and ceramide subunits (Scriver C R, Beaudet A L, Valle D, Sly W S. The metabolic and molecular basis of inherited disease. 8th ed. New York: McGraw-Hill Pub, 2001: 3635-3668). When the enzyme is defective, the substance accumulates, particularly in cells of the mononuclear cell lineage, and organs and tissues including the spleen, liver, kidneys, lungs, brain and bone marrow.

There are two major forms: non-neuropathic (type 1, most commonly observed type in adulthood) and neuropathic (type 2 and 3). GBA (GBA glucosidase, beta, acid), the only known human gene responsible for glucosidase-mediated GD, is located on chromosome 1, location 1q21. More than 200 mutations have been defined within the known genomic sequence of this single gene (NCBI Reference Sequence: NG_009783.1). The most commonly observed mutations are N370S, L444P, RecNcil, 84GG, R463C, recTL and 84 GG is a null mutation in which there is no capacity to synthesize enzyme. However, N370S mutation is almost always related with type 1 disease and milder forms of disease. Very rarely, deficiency of sphingolipid activator protein (Gaucher factor, SAP-2, saposin C) may result in GD. In some embodiments, triplex-forming molecules are used to induce recombination of donor oligonucleotides designed to correct mutations in GBA.

In another embodiment, compositions and the methods disclosed herein are used to treat Fabry disease (also known as Fabry's disease, Anderson-Fabry disease, angiokeratoma corporis diffusum and alpha-galactosidase A deficiency), a rare X-linked recessive disordered, resulting from a deficiency of the enzyme alpha galactosidase A (a-GAL A, encoded by GLA). The human gene encoding GLA has a known genomic sequence (NCBI Reference Sequence: NG_007119.1) and is located at Xp22 of the X chromosome. Mutations in GLA result in accumulation of the glycolipid globotriaosylceramide (abbreviated as Gb3, GL-3, or ceramide trihexoside) within the blood vessels, other tissues, and organs, resulting in impairment of their proper function (Karen, et al., Dermatol. Online J., 11 (4): 8 (2005)). The condition affects hemizygous males (i.e. all males), as well as homozygous, and potentially heterozygous (carrier), females. Males typically experience severe symptoms, while women can range from being asymptomatic to having severe symptoms. This variability is thought to be due to X-inactivation patterns during embryonic development of the female. In some embodiments, triplex-forming molecules are used to induce recombination of donor oligonucleotides designed to correct mutations in GLA.

In preferred embodiments, the disclosed compositions and methods are used to treat Hurler syndrome (HS). Hurler syndrome, also known as mucopolysaccharidosis type I (MPS I), a-L-iduronidase deficiency, and Hurler's disease, is a genetic disorder that results in the buildup of mucopolysaccharides due to a deficiency of a-L iduronidase, an enzyme responsible for the degradation of mucopolysaccharides in lysosomes (Dib and Pastories, Genet. Mol. Res., 6(3):667-74 (2007)). MPS I is divided into three subtypes based on severity of symptoms. All three types result from an absence of, or insufficient levels of, the enzyme a-L-iduronidase. MPS I H or Hurler syndrome is the most severe of the MPS I subtypes. The other two types are MPS I S or Scheie syndrome and MPS I H-S or Hurler-Scheie syndrome. Without α-L-iduronidase, heparan sulfate and dermatan sulfate, the main components of connective tissues, build-up in the body. Excessive amounts of glycosaminoglycans (GAGs) pass into the blood circulation and are stored throughout the body, with some excreted in the urine. Symptoms appear during childhood, and can include developmental delay as early as the first year of age. Patients usually reach a plateau in their development between the ages of two and four years, followed by progressive mental decline and loss of physical skills (Scott et al., Hum. Mutat. 6: 288-302 (1995)). Language may be limited due to hearing loss and an enlarged tongue, and eventually site impairment can results from clouding of cornea and retinal degeneration. Carpal tunnel syndrome (or similar compression of nerves elsewhere in the body) and restricted joint movement are also common.

a. Exemplary Target Sites

The human gene encoding alpha-L-iduronidase (α-L-iduronidase; IDUA) is found on chromosome 4, location 4p16.3, and has a known genomic sequence (NCBI Reference Sequence: NG_008103.1). Two of the most common mutations in IDUA contributing to Hurler syndrome are the Q70X and the W420X, non-sense point mutations found in exon 2 (nucleotide 774 of genomic DNA relative to first nucleotide of start codon) and exon 9 (nucleotide 15663 of genomic DNA relative to first nucleotide of start codon) of IDUA respectively. These mutations cause dysfunction alpha-L-iduronidase enzyme. Two triplex-forming molecule target sequences including a polypurine:polypyrimidine stretches have been identified within the IDUA gene. One target site with the polypurine sequence 5′ CTGCTCGGAAGA 3′ (SEQ ID NO:141) and the complementary polypyrimidine sequence 5′ TCTTCCGAGCAG 3′ (SEQ ID NO: 142) is located 170 base pairs downstream of the Q70X mutation. A second target site with the polypurine sequence 5′ CCTTCACCAAGGGGA 3′ (SEQ ID NO:143) and the complementary polypyrimidine sequence 5′ TCCCCTTGGTGAAGG 3′ (SEQ ID NO: 144) is located 100 base pairs upstream of the W402X mutation. In preferred embodiments, triplex-forming molecules are designed to bind/hybridize in or near these target locations.

b. Exemplary Triplex Forming Sequences and Donors

i. W402X Mutation

In some embodiments, a triplex-forming molecule binds to the target sequence upstream of the W402X mutation includes the nucleic acid sequence TTCCCCT (SEQ ID NO: 145), preferable includes the sequence TTCCCCT (SEQ ID NO: 145) linked to the sequence TCCCCTT (SEQ ID NO: 146), or more preferable includes the sequence TTCCCCT (SEQ ID NO: 145) linked to the sequence TCCCCTTGGTGAAGG (SEQ ID NO: 147).

In some preferred embodiments, the triplex forming nucleic acid is a peptide nucleic acid that binds to the target sequence upstream of the W402X mutation including the sequence TTJJJJT (SEQ ID NO: 148), linked to the sequence TCCCCTT (SEQ ID NO: 146) or TCCCCTTGGTGAAGG (SEQ ID NO: 147), optionally, but preferably wherein one or more of the PNA monomers is a γPNA.

In specific embodiments, the triplex forming nucleic acid is a peptide nucleic acid having the sequence Lys-Lys-Lys-TTJJJJT-OOO-TCCCCTTGGTGAAGG-Lys-Lys-Lys (SEQ ID NO:159) (IDUA402tc715) optionally, but preferably wherein one or more of the PNA monomers is a γPNA. In even more specific embodiments, the bolded and underlined residues are miniPEG-containing γPNA.

In the most preferred embodiments, triplex-forming molecules are administered according to the disclosed methods in combination with one or more donor oligonucleotides designed to correct the point mutations at Q70X or W402X mutations sites. In some embodiments, in addition to containing sequence designed to correct the point mutation at Q70X or W402X mutation, the donor oligonuclotides may also contain 7 to 10 additional, synonymous (silent) mutations. The additional silent mutations can facilitate detection of the corrected target sequence using allele-specific PCR of genomic DNA isolated from treated cells.

In some embodiments, the donor oligonucleotide with the sequence 5′ AGGACGGTCCCGGCCTGCGACACTTCCGCCCATAATTGTTCTT CATCTGCGGGGCGGGGGGGGG 3′ (SEQ ID NO:149), or a functional fragment thereof that is suitable and sufficient to correct the W402X mutation is administered with triplex-forming molecules designed to target the binding site upstream of W402X to correct the W402X mutation in cells.

ii. Q70X Mutation

In some embodiments, a triplex-forming molecule that binds to the target sequence downstream of the Q70X mutation includes the nucleic acid sequence CCTTCT (SEQ ID NO:150), preferable includes the sequence CCTTCT (SEQ ID NO: 150) linked to the sequence TCTTCC (SEQ ID NO:151), or more preferable includes the sequence CCTTCT (SEQ ID NO: 150) linked to the sequence TCTTCCGAGCAG (SEQ ID NO: 152).

In preferred embodiments, the triplex forming nucleic acid is a peptide nucleic acid that binds to the target sequence downstream of the Q70X mutation including the sequence JJTTJT (SEQ ID NO:162) linked to the sequence TCTTCC (SEQ ID NO:151) or TCTTCCGAGCAG (SEQ ID NO:152) optionally, but preferably wherein one or more of the PNA monomers is a γPNA.

In a specific embodiment, a tcPNA with a sequence of Lys-Lys-Lys-JJTTJT-OOO-TCTTCCGAGCAG-Lys-Lys-Lys (SEQ ID NO:153) (IDUA402tc715) optionally, but preferably wherein one or more of the PNA monomers is a γPNA. In even more specific embodiments, the bolded and underlined residues are miniPEG-containing γPNA.

A donor oligonucleotide can have the sequence 5′GGGACGGCGCCCACATAGGCCAAATTCAATTGCTGATCCCAGCT TAAGACGTACTGGTCAGCCTGGC 3′ (SEQ ID NO:154), or a functional fragment thereof that is suitable and sufficient to correct the Q70X mutation is administered with triplex-forming molecules designed to target the binding site downstream of Q70X to correct the of Q70X mutation in cells.

### X. Combination Therapies

Each of the different components of gene editing and potentiation disclosed here can be administered alone or in any combination and further in combination with one or more additional active agents. In all cases, the combination of agents can be part of the same admixture, or administered as separate compositions. In some embodiments, the separate compositions are administered through the same route of administration. In other embodiments, the separate compositions are administered through different routes of administration.

A. Conventional Therapeutic Agents

Examples of preferred additional active agents include other conventional therapies known in the art for treating the desired disease or condition. For example, in the treatment of sickle cell disease, the additional therapy may be hydroxurea.

In the treatment of cystic fibrosis, the additional therapy may include mucolytics, antibiotics, nutritional agents, etc. Specific drugs are outlined in the Cystic Fibrosis Foundation drug pipeline and include, but are not limited to, CFTR modulators such as KALYDECO® (invascaftor), ORKAMBI™ (lumacaftor+ivacaftor), ataluren (PTC124), VX-661+invacaftor, riociguat, QBW251, N91115, and QR-010; agents that improve airway surface liquid such as hypertonic saline, bronchitol, and P-1037; mucus alteration agents such as PULMOZYME® (dornase alfa); anti-inflammatories such as ibuprofen, alpha 1 anti-trypsin, CTX-4430, and JBT-101; anti-infective such as inhaled tobramycin, azithromycin, CAYSTON® (aztreonam for inhalation solution), TOBI inhaled powder, levofloxacin, ARIKACE® (nebulized liposomal amikacin), AEROVANC® (vancomycin hydrochloride inhalation powder), and gallium; and nutritional supplements such as aquADEKs, pancrelipase enzyme products, liprotamase, and burlulipase.

In the treatment of HIV, the additional therapy maybe an antiretroviral agents including, but not limited to, a non-nucleoside reverse transcriptase inhibitor (NNRTIs), a nucleoside reverse transcriptase inhibitor (NRTIs), a protease inhibitors (PIs), a fusion inhibitors, a CCR5 antagonists (CCR5s) (also called entry inhibitors), an integrase strand transfer inhibitors (INSTIs), or a combination thereof.

In the treatment of lysosomal storage disease, the additional therapy could include, for example, enzyme replacement therapy, bone marrow transplantation, or a combination thereof.

B. Additional Mutagenic Agents

The compositions can be used in combination with other mutagenic agents. In a preferred embodiment, the additional mutagenic agents are conjugated or linked to gene editing technology or a delivery vehicle (such as a nanoparticle) thereof. Additional mutagenic agents that can be used in combination with gene editing technology, particularly triplex forming molecules, include agents that are capable of directing mutagenesis, nucleic acid crosslinkers, radioactive agents, or alkylating groups, or molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked molecules as described in PCT/US/94/07234 by Yale University.

It may also be desirable to administer gene editing compositions in combination with agents that further enhance the frequency of gene modification in cells. For example, the disclosed compositions can be administered in combination with a histone deacetylase (HDAC) inhibitor, such as suberoylanilide hydroxamic acid (SAHA), which has been found to promote increased levels of gene targeting in asynchronous cells.

The nucleotide excision repair pathway is also known to facilitate triplex-forming molecule-mediated recombination. Therefore, the disclosed compositions can be administered in combination with an agent that enhances or increases the nucleotide excision repair pathway, for example an agent that increases the expression, or activity, or localization to the target site, of the endogenous damage recognition factor XPA.

Compositions may also be administered in combination with a second active agent that enhances uptake or delivery of the gene editing technology. For example, the lysosomotropic agent chloroquine has been shown to enhance delivery of PNAs into cells (Abes, et al., J. Controll. Rel., 110:595-604 (2006). Agents that improve the frequency of gene modification are particularly useful for in vitro and ex vivo application, for example ex vivo modification of hematopoietic stem cells for therapeutic use.

### XI. Methods for Determining Triplex Formation and Gene Modification

A. Methods for Determining Triplex Formation

A useful measure of triple helix formation is the equilibrium dissociation constant, K_{d}, of the triplex, which can be estimated as the concentration of triplex-forming molecules at which triplex formation is half-maximal. Preferably, the molecules have a binding affinity for the target sequence in the range of physiologic interactions. Preferred triplex-forming molecules have a K_{d} less than or equal to approximately 10⁻⁷ M. Most preferably, the K_{d} is less than or equal to 2×10⁻⁸ M in order to achieve significant intramolecular interactions. A variety of methods are available to determine the K_{d} of triplex-forming molecules with the target duplex. In the examples which follow, the K_{d} was estimated using a gel mobility shift assay (R. H. Durland et al., Biochemistry 30, 9246 (1991)). The dissociation constant (K_{d}) can be determined as the concentration of triplex-forming molecules in which half was bound to the target sequence and half was unbound.

B. Methods for Determining Gene Modification

Sequencing and allele-specific PCR are preferred methods for determining if gene modification has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example by sequencing, allele-specific PCR, or restriction endonuclease selective PCR (REMS-PCR); analysis of mRNA transcribed from the target gene for example by Northern blot, in situ hybridization, real-time or quantitative reverse transcriptase (RT) PCT; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by the target gene. For example, if the target gene encodes an enzyme, an assay designed to test enzyme function may be used.

### XII. Kits

Medical kits are also disclosed. The medical kits can include, for example, a dosage supply of gene editing technology or a potentiating agent thereof, or a combination thereof in separately or together in the same admixture. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. The kits can include printed instructions for administering the compound in a use as described above.

### EXAMPLES

### Example 1: Triplex-Forming PNA Molecules can Modify F508del CFTR

### Materials and Methods

Oligonucleotides

PNAs with an 8-amino-2,6-dioxaoctanoic acid linker were purchased from Bio-Synthesis (Lewisville Tex.) or Panagene (Daejeon, Korea) and purified by HPLC. Donor oligonucleotides 50 nt in length were synthesized by Midland Certified Reagent (Midland Tex.), 5′- and 3′-end protected by three phosphorothioate internucleoside linkages at each end and purified by reversed phase-HPLC. Sequences of PNA molecules used are given in FIGS. 1A-1E.

Human donor DNA sequence:
(SEQ ID NO: 96)
5′ TTCTGTATCTATATTCATCATAGGAAACACCAA
AGATAATGTTCTCCTTAATGGTGCCAGG 3′
Mouse donor DNA sequence:
(SEQ ID NO: 169)
5′ TCTTATATCTGTACTCATCATAGGAAACACCAA
AGATAATGTTCTCCTTGATAGTACCCGG 3′

In the mismatched PNA control experiments, a PNA molecule targeting the human β-globin gene was used with 12 mismatches in the Watson Crick domain relative to the CF PNA2:

β-globin-targeted PNA
(SEQ ID NO: 33)
JTTTJTTTJTJT-OOO-TCTCTTTCTTTCAGGGCA-
CFTR-targeted PNA
(SEQ ID NO: 93)
TJTJJTTT-OOO-TTTCCTCTATGGGTAAG-

The β-globin-targeted PNA has 8 T, 5 C, 2 A, 3 G in the Watson-Crick domain and 8 T and 4 J in the Hoogsteen domain.

The CFTR-targeted PNA has 7 T, 3 C, 3 A, 4 G in the Watson-Crick domain and 5 T and 3 J in the Hoogsteen domain.

Gel Shift Assays for PNA Binding

To test the binding of candidate tail-clamp PNA molecules to the targeted site in the CFTR gene, PNA was incubated with plasmid DNA containing the target site at 37° C. overnight, with 10 μM KCl in TE at final volume of 10 μL. Samples were digested with restriction enzymes flanking the binding site (EcoRI and BamHI), and the products run on an 8% non-denaturing PAGE gel. A silver stain was used to visualize the products.

Nanoparticle Formulation

PLGA nanoparticles loaded with PNA and DNA were formulated and characterized using a double-emulsion solvent evaporation technique as previously described (McNeer, et al., Mol Ther., 19:172-180 (2011)). Instead of 1:1 PNA:DNA, 1:2 PNA:DNA was loaded in each batch in initial screening studies (20 μL of 2 mM donor, 20 μL of 1 mM PNA per 80 mg of PLGA). For particles in subsequent studies, 80 nmole (40 uL of 2 mM solution) of PNA and 40 nmole (20 uL of 2 mM solution) of DNA were used per 80 mg particle batch (scaled up or down accordingly).

Briefly, 80 mg of polymer was dissolved in 160 uL dichloromethane overnight. PNA and DNA were dissolved in RNase/DNase free water. The PNA and DNA were then added dropwise into the dissolved polymer while vortexing, then sonicated for 10 seconds three times (Tekmar Probe Sonicator, Cincinnati, Ohio). The polymer solution was then added dropwise to 3.2 mL of 5% poly (vinyl alcohol) (PVA) while vortexing, then sonicated for 10 seconds three times using a probe sonicator on ice. The emulsion was then transferred to 20 mL of 0.3% PVA in a beaker with a stir bar, and left for 3 hours to let the solvent evaporate. This solution was then washed three times by ultracentrifugation with 10 mL of water, then resuspended in 2.5 mL of water and transferred into to Eppendorf tubes. Eppendorfs were frozen at −80° C. for at least 2 hours, then transferred to a lyophilizer for 3 days.

Cell Culture

CFBE cells (CFBE41o-) and human bronchial epithelial cells (16HBE14o-) (Gruenert, et al., Official Journal of the European Cystic Fibrosis Society, 3 (Suppl 2):191-196 (2004)) were grown with LHC-8 media (Invitrogen) with 10% FBS, 1X antibiotic antimycotic (Gibco), and tobramycin 40 mg per 500 mL (Sigma). Once grown to confluence, cells were trypsinized by first washing with 0.05% trypsin, then adding 0.25% trypsin for 5 minutes, and harvesting with RPMI medium with 10% FBS. Cells were frozen in 5% DMSO in culture medium as necessary. Nanoparticles were resuspended in culture media by vigorous vortexing and water sonication, then added directly to cells at concentrations of 2 mg/mL/1×10\hat{ }6 cells (corresponding to approximately 10\hat{ }9 PNA/DNA molecules delivered to each cell assuming 100% efficiency).

To test primers, a 712 base pair region of the CFTR gene, with either the F508DEL or corrected sequence (including silent modifications), was cloned into plasmids. PCR reactions were first tested on plasmids. Gradient and step-down PCR at varying conditions was performed to ensure that F508del primers only amplified the F508del plasmid, and the donor-specific primers only amplified the donor-sequence-containing plasmids.

Genomic DNA Extraction and AS-PCR

Genomic DNA was harvested from cells and purified using the Wizard Genomic DNA Purification kit (Promega, Madison Wis.). Equal amounts of genomic DNA from each sample were subjected to allele-specific PCR, with a gene-specific reverse primer, and an allele-specific forward primer in which the 3′ end corresponds to the 6 bp modified sequence. Quantitative PCR was performed using a Stratagene Mx 3000P cycler. 0.2 μM donor DNA was used in spiking experiments. Copy numbers of DNA in the PCR reaction were approximately 10\hat{ }14 copies of genomic DNA and 10\hat{ }12 copies of spiked donor DNA. PCR products were separated on a 1% agarose gel and visualized using a gel imager. Relative gene modification was calculated using the 2-ΔΔCt method, with the average of the untreated controls used as the reference groups 51.

AS-PCR conditions are as follows. Platinum Taq polymerase (Invitrogen, Carlsbad Calif.) was used for PCR reactions: 5 uL betaine, 4.25 uL water, 2.5 uL 10× Platinum Taq PCR buffer, 1.25 uL 50 mM MgCl2, 0.5 uL dNTPs, 0.5 uL each primer at 10 uM, 0.5 uL Platinum Taq polymerase, and 10 uL of genomic DNA at 40 ng/uL. PCR cycler conditions for human CFTR were as follows: 95° C. 2 min, 94° C. 30 sec, 69° C. 1 min, 72° C. 1 min, 94° C. 30 sec, 68° C. 1 min, 72° C. 1 min, 94° C. 30 sec, 67° C. 1 min, 72° C. 1 min, 94° C. 30 sec, 66° C. 1 min, 72° C. 1 min, 94° C. 30 sec, 65° C. 1 min, 72° C. 1 min, [94° C. 30 sec, 65° C. 1 min, 72° C. 1 min]×35 cycles, 72° C. 2 min, hold at 4° C. 1. PCR cycler conditions for mouse CFTR were as follows: 94° C. for 5 min, [94° C. 30 sec 66.9° C. (for detection of F508del) or 68.3° C. (for detection of modification) 45 sec, 72° C. 1 min]×40, 72° C. 6 min, hold at 4° C. Conditions were optimized using plasmids containing the target sequences as indicated above. Of note, donor sequences contained an additional 4 base-pairs of silent mutations distinguishing the donor sequence from wild-type CFTR, to ensure that contaminating wild-type cells (environmental or from other cell cultures) do not appear as false-positives.

For regular sequencing, High Fidelity Platinum Taq Polymerase (Invitrogen, Carlsbad Calif.) was used. PCR conditions for production of amplicons for regular sequencing were as follows: 0.5 uL dNTPs, 2.5 uL 10x HiFi Buffer, 1.5 uL 50 mM MgCl2, 14.1 uL water, 0.4 uL Taq HiFi, 0.5 uL each primer at 10 uM, 5 uL genomic DNA at 80 ng/uL. PCR cycler conditions were as follows: 94° C. 2 min, [94° C. 30 sec 55° C. 45 sec 68° C. 1 min]×35, 68° C. 1 min, hold at 4° C.

### Results

A donor DNA molecule homologous to the targeted region containing the F508del sequence, and three tail-clamp PNA molecules that bind near this site at homopurine/homopyrimidine stretches were designed (FIGS. 1A-1E). A gel shift binding assay was used to confirm binding of these PNA molecules to the desired targets—successful binding is indicated by presence of a DNA band more proximally on the gel as the bound triplex-forming PNA molecule slows down the transit of the complex. Several bands may be present due to different binding configurations, as previously described (Nielsen, et al., Horizon Bioscience, Wymondham (2004)).

An AS-PCR assay was designed to differentiate between the integrated donor sequence and the endogenous F508del sequence, using plasmids for optimization and validation of the allele specificity of the PCR reaction. Primers specific to the donor DNA selectively amplified the plasmid containing the donor sequence, whereas primers specific to the F508del sequence only amplified the plasmid containing the F508del sequence. Importantly, spiking of the PCR reaction on genomic DNA with excess donor DNA or excess PNA did not lead to a false positive PCR artifact. However, spiking of the PCR reaction with donor DNA and PNA at high doses did result in inhibition of the PCR reaction, indicating that the AS-PCR may not pick up all samples with corrected genomes. Occasional amplification of the F508del sequence with donor primers was also observed, which would not lead to false positives or negatives when trying to detect the donor sequence. Because of these limitations, AS-PCR was only used as an initial screening tool to identify active molecules before moving to sequencing and functional studies.

To screen PNA molecules for gene editing activity, PLGA nanoparticles were loaded with PNAs and donor DNAs using a double emulsion solvent evaporation technique as previously described (McNeer, et al., Mol Ther., 19:172-180 (2011)). Nanoparticles with the donor DNAs alone, or DNAs and the various PNA molecules, were then tested on CF bronchial epithelial (CFBE) cells containing F508del (CFBE41o-) (Gruenert, et al., Official Journal of the European Cystic Fibrosis Society, 3 (Suppl 2):191-196 (2004)). AS-PCR showed that F508del cells treated with PLGA nanoparticles containing both donor DNAs and hCFPNA2 had the desired modification present. Nanoparticles with donor DNA alone or with donor DNA plus either hCFPNA1 or hCFPNA3 were not effective.

### Example 2: The CFTR Gene is Modified in Isolated Clones

### Materials and Methods

RNA Extraction and Reverse-Transcription AS-PCR

RNAeasy Plus Qiagen Kit (Gaithersburg, Md.) was used to extract RNA, and Invitrogen superscript III kit (Carlsbody, Calif.) was used to make cDNA. PCR reactions contained cDNA, 20% Betaine, 0.2 mM dNTPs, Advantage 2 Polymerase Mix, 0.2 μM of each primer, and 2% platinum taq.

Gene-specific reverse primer:
(SEQ ID NO: 170)
5′ CCTAGTTTTGTTAGCCATCAGTTTACAGAC 3′
F508DEL CF primer:
(SEQ ID NO: 171)
5′ GCCTGGCACCATTAAAGAAAATATCATTGG 3′
Primer for corrected/donor:
(SEQ ID NO: 66)
5′ CCTGGCACCATTAAGGAGAACATTATCTT 3′

PCR cycler conditions were as follows: 95° C. 5 min, [95° C. 30 sec 65° C. 1 min 72° C. 1 min]×35, 72° C. 5 min, hold at 4° C.

Deep Sequencing

Genomic DNA was isolated from treated cells or mouse tissue, and PCR reactions performed with high fidelity TAQ polymerase. Each PCR tube consisted of 28.2 μL dH2O, 5 μL 10× HiFi Buffer, 3 μL 50 mM MgCl2, 1 μL DNTP, 1 μL each of forward and reverse primer, 0.8 μL HiFi Platinum Taq and 10 μL DNA template. Separate barcoded primers (6 bp barcode plus primer) were used for each sample. PCR conditions were as follows: For regular sequencing, High Fidelity Platinum Taq Polymerase (Invitrogen, Carlsbad Calif.) was used. PCR conditions for production of amplicons for regular sequencing were as follows: 0.5 uL dNTPs, 2.5 uL 10× HiFi Buffer, 1.5 uL 50 mM MgCl2, 14.1 uL water, 0.4 uL Taq HiFi, 0.5 uL each primer at 10 uM, 5 uL genomic DNA at 80 ng/uL. PCR cycler conditions were as follows: 94° C. 2 min, [94° C. 30 sec 55° C. 45 sec 68° C. 1 min]×35, 68° C. 1 min, hold at 4° C. PCR products were prepared by end-repair and adapter ligation according to Illumina protocols (San Diego, Calif.), and pooled samples sequenced by the Illumina HiSeq with 75 paired-end reads at the W.M. Keck Facility at Yale University.

Analysis was performed using PERL file and software available through a Yale University website. The program Btrim was used to trim off low-quality regions of each read and to assign the trimmed reads to each barcode (Btrim, Genomics, 98:152-153 (2011)). The number of reads with modified sequence or original sequence were also searched by using Btrim. For off-target sites, the trimmed reads were mapped using program bowtie253.

MQAE Assay for Chloride Flux

N-[ethoxycarbonylmethyl]-6-methoxy-quinolinium bromide (MQAE) is a chloride sensitive fluorescent dye used to assess chloride flux in plated CFBE cells as previously described (Shenoy, et al., Pediatric Research, 70:447-452 (2011)). Cells were grown to confluence directly on coverslips. Then, cells were placed in Cl-containing solution (135 mM NaCl, 5 mM KCl, 1 mM CaCl2, 1.2 mM MgSO4, 2 mM NaH2PO4, 2 mM HEPES, and 10 mM glucose), then moved to a chloride free solution (135 mM NaCyclamate, 3 mM KGluconate, 0.5 mM CaCyclamate, 1.2 mM MgSO4, 2 mM KH2PO4, 2 mM HEPES, 10 mM glucose). Finally, chloride flux was assessed in solution with forskolin (10 μM) and IBMX (100 μM) added. MQAE experiments were performed on an Olympus IX-71 inverted microscope, with MQAE excited at 354 nm and fluorescence measured at 460 nm every 5 s. Fluorescence was measured on a cell-by-cell basis, with 30 to 100 cells catalogued per slide. The rate of change in MQAE fluorescence (arbitrary fluorescence units AFU/time) was graphed, and AFU/min was compared between groups. Graphs shown are normalized to background.

### Results

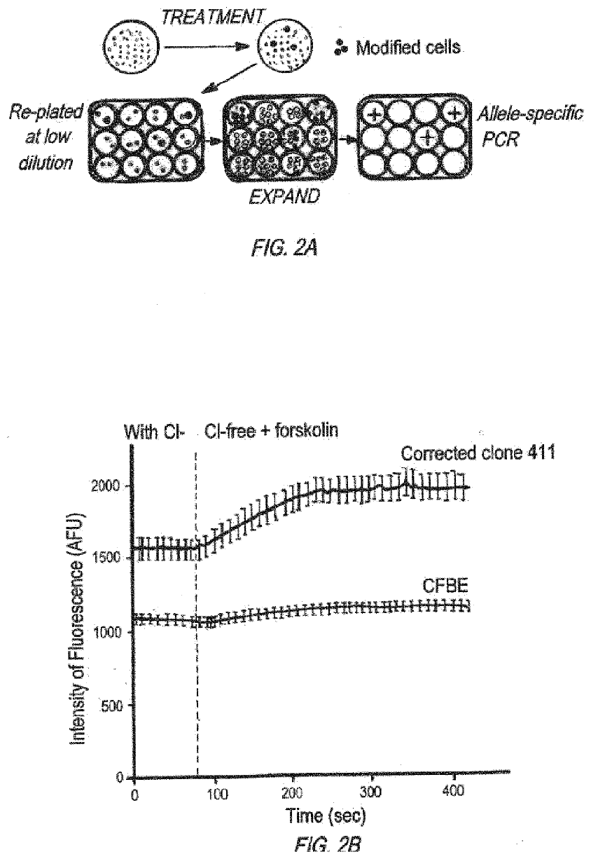

The nanoparticle-treated cell populations were seeded at limiting dilution into 96-well plates, and expanded to isolate clones positive for the modification (FIG. 2A, Table 1).

TABLE 1
Frequency of modification calculated
using limiting dilution analysis.

| Cell Concentration | Number of Wells | Number Positive |
|---|---|---|
| 20/well | 192 | 19 |
| 10/well | 192 | 15 |
| 1/well | 192 | 6 |

Percent Modification: 0.7%
95% CI: 0.5%-0.96%

The frequency of modification in cells treated once with PLGA nanoparticles containing hCFPNA2 and the donor DNA, as calculated by limiting dilution analysis (Hu, et al., J Immunol Methods, 347:70-78 (2009)), was 0.5-0.96%.

Populations positive for CFTR gene correction were expanded by repeated limiting dilution to create more homogeneous clones, with the modification persisting over months of cell expansion. A 700 base-pair region around the modification site was amplified by PCR and sequenced, confirming the presence of the corrected sequence in clone 411, and regular sequencing with limited PCR cycles revealed heterozygosity of the sample although with low sequencing quality. Higher quality reads were obtained by deep-sequencing, which revealed that clone 411 was indeed heterozygous. Clone 411 was found to have 15897/35178 (45%) of alleles with the modified sequence, implying a heterozygous population with possibly a few contaminating unmodified cells (which may have remained even after the limiting dilution cell isolation process).

A region of an unrelated gene that has homology to the hCFPNA2 binding site, except for one base-pair mismatch, adenylate cyclase type 4 on chromosome 14, was also sequenced, and no mutations were identified in 96 sequenced clones. While this regular sequencing in clones would not be able to identify mutations at a frequency lower than 1/96, additional experiments to ascertain off-target effects were performed in treated cells (see below). Correction of the CFTR gene was also confirmed using reverse transcriptase, allele-specific PCR on RNA extracted from a positive clone, as seen by the band corresponding to the modified sequence.

Chloride efflux in the positive clones was quantified using MQAE, a fluorescent indicator dye, and perfusate solutions that switched from chloride containing solutions to chloride free solutions in the presence of forskolin and IBMX to maximally activate functional CFTR at the cell surface (Shenoy, et al., Pediatric Research, 70:447-452 (2011); Egan, et al., Nat Med., 8:485-492 (2002)). While untreated cells had minimal chloride efflux (flat line), the positive clones had increased chloride efflux in individually tested cells (FIG. 2B). The increased chloride efflux was calculated by measuring the rate of change in fluorescence over time (ΔAFU/Δsec) as perfusate solutions were changed from chloride containing to chloride free solutions in the presence of a CFTR stimulating cocktail. Chloride efflux was found to be significantly increased in the positive clones (FIG. 2C). Efflux rates of HBE cells (p<0.0001) and clone 105 (p=0.0061) and clone 411 (p<0.0001) were significantly different from that of untreated CF cells. There was no difference in chloride efflux between untreated cells and those treated with blank particles. One way ANOVA with multiple comparisons was used to analyze chloride efflux in untreated CF cells, blank particle treated CF cells, clone 105, clone 411 and normal human bronchial epithelial cells (16HBE14o-). In sum, chloride efflux in clones was found to be similar to efflux in wild-type human bronchial epithelial (HBE) cells, although there was some variation between clones. For instance, “clone” 105, which had lower response, was found to have 350/8346168 of alleles modified in one deep sequencing run, indicating a heterogeneous population with variable expansion of modified cells.

### Example 3: PLGA/PBAE/MPG Nanoparticles have Improved In Vivo Activity

### Materials and Methods

Nanoparticle Formulation and Characterization

Poly(beta amino ester) (PBAE) was synthesized by a Michael addition reaction of 1,4-butanediol diacrylate (Alfa Aesar Organics, Ward Hill, Mass.) and 4,4′-trimethylenedipiperidine (Sigma, Milwaukee, Wis.) as previously reported (Akinc, et al., Bioconjug Chem., 14:979-988 (2003)). DSPE-PEG(2000)-maleimide was purchased from Avanti Polar Lipids (Alabaster, Ala.). MPG peptides were purchased from Keck (Yale University). CPPs were covalently linked to DSPE-PEG-maleimide as previously reported (Fields, et al., J Control Release (2012)), PLGA/PBAE particles contained 15% PBAE (wt %), and solvent from these particles was evaporated overnight in PVA instead of for three hours as above. To make surface-modified particles, DSPE-PEG-MPG was added to the 5.0% PVA solution during formation of the second emulsion at a 5 nmol/mg ligand-to-polymer ratio.

In subsequent studies, particles were loaded as indicated. SEM imaging and controlled release studies were performed as before (McNeer, et al., Mol Ther., 19:172-180 (2011)). Briefly, for SEM imaging, particles were sputter coated with gold prior to imaging. For controlled release studies, particles were dissolved in 600 uL of DNase/RNase free water, put in a 37° C. shaker, and at set timepoints centrifuged at 13000 RPM in a microfuge; at each timepoint, the supernatant was examined using a NanoDrop 8000 for nucleic acid content.

### Results

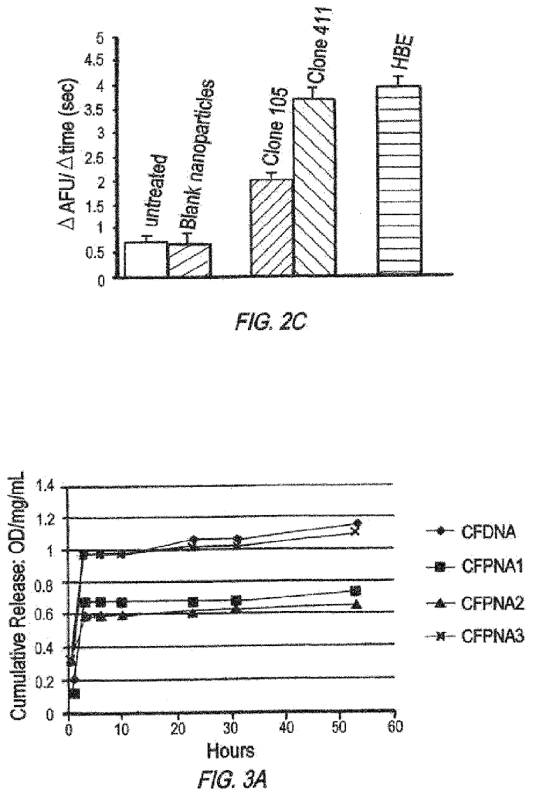

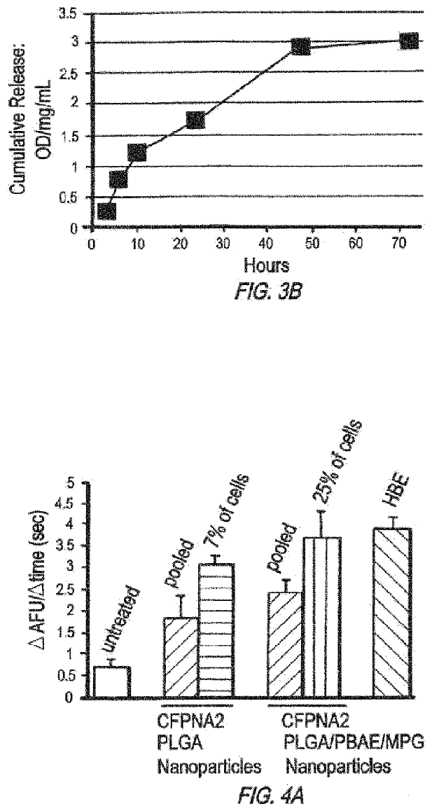

Nanoparticles were then formulated from a blend of PLGA and 15% (wt %) poly (beta amino ester) (PBAE), surface modified with the nuclear-localization sequence-containing cell-penetrating peptide MPG (modified PLGA/PBAE/MPG nanoparticles) (Fields, et al., J Control Release (2012)). Particles exhibited uniform size and morphology on SEM, and released most of their contents quickly, within the first 6-12 hours of incubation in PBS at 37° C., although there was more sustained release of nucleic acid cargo using the modified nanoparticles (FIGS. 3A and 3B). Increased uptake of fluorescently-labeled PNA molecules was seen when PLGA/PBAE/MPG nanoparticles were used on human CFBE cells.

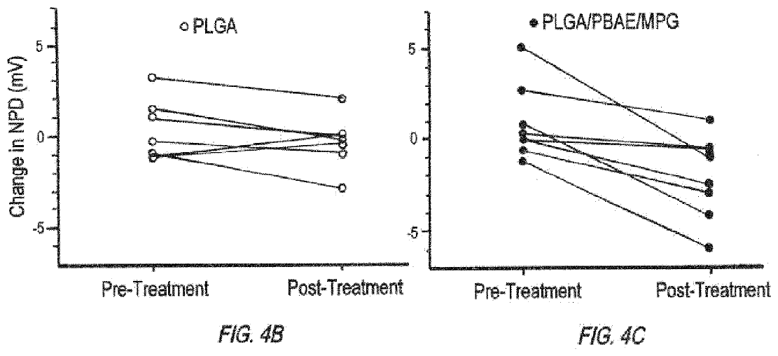

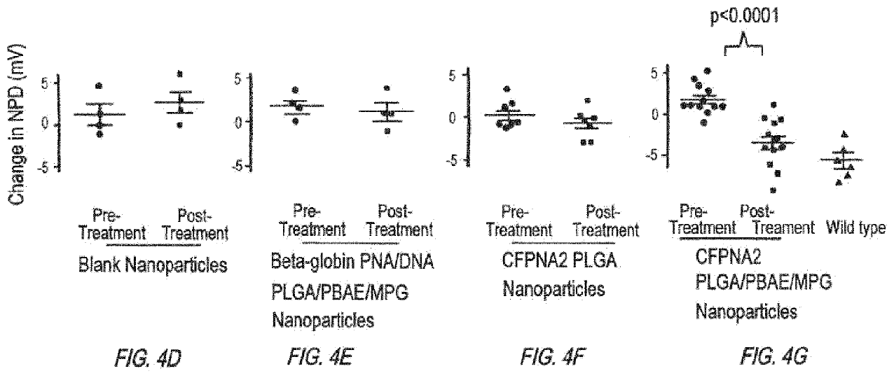

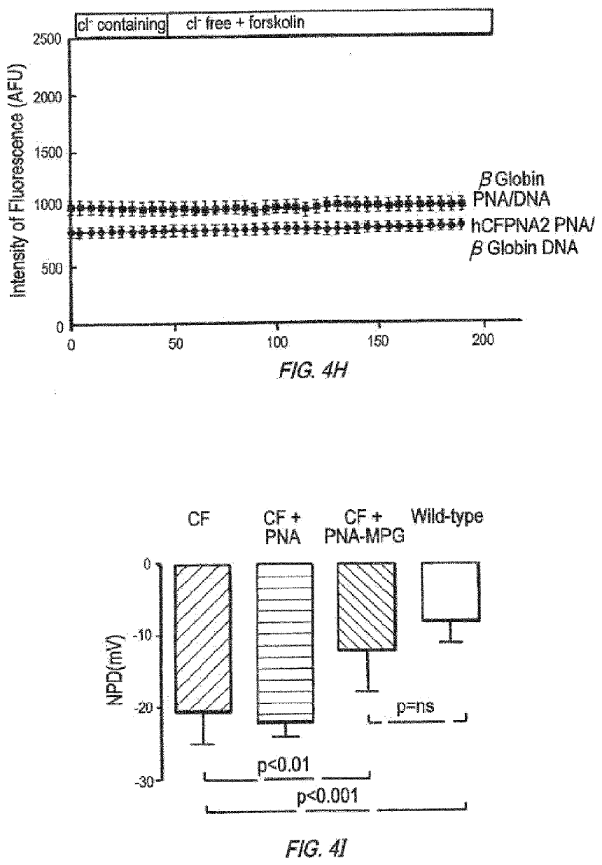

Change in chloride efflux was seen in CFBE cells serially treated three times with nanoparticles, without isolation of positive cells (FIG. 4A). F508del CFBE cells were plated at 10% confluence, then treated 3 times with 2 mg/mL particles over 7 days. They were then replated on slides and allowed 7-10 days to grow to confluence before the MQAE assay was performed to determine chloride efflux. Of note, interrogation of individual cells in these studies allowed quantification of the absolute number of cells with functional chloride efflux. Approximately 7% of the PLGA-nanoparticle treated cells demonstrated efflux similar to positive controls, and when CFBE cells were treated repeatedly with modified PLGA/PBAE/MPG nanoparticles 25% of cells demonstrated efflux equivalent to positive controls; this difference in modification efficiency was statistically significant (p=0.003 two-tailed Fisher's exact test). Cells treated similarly with PNA-carrying nanoparticles targeting a non-related genomic target or hCFPNA2 with a different donor DNA targeting a non-related genomic target did not have any change in chloride efflux (FIG. 4D). Previous work indicated that this modified nanoparticle formulation is also optimal for in vivo delivery of cargo to the respiratory epithelium. PLGA/PBAE/MPG nanoparticles are taken up by both macrophages and lung epithelial cells in mice (Fields, et al., Advanced Healthcare Materials, 2014 (2014)).

### Example 4: Correction of Murine F508del In Vivo

### Materials Methods

Animal Model

A mouse model homozygous for the F508DEL mutation on a fully backcrossed C57/BL6 background was used (Zeiher, et al., The Journal of Clinical Investigation, 96:2051-2064 (1995)). Mice were between 12 and 40 weeks of age (the majority between 3 and 6 months of age), an equal mix of male and female. Nanoparticles were resuspended at 1 mg in 50 μL PBS, sonicated and administered to mice by intranasal instillation. Mice were treated with a total of 7 mg of nanoparticles over a course of 2 weeks (one treatment every other day)—this corresponds to a total of approximately 3.5 nmoles of donor DNA (˜10\hat{ }15 copies) and 7 nmoles of PNA per mouse (˜2×10\hat{ }15 copies). Estimating about 400 million cells/mouse lung, this corresponds to approximately 5 million PNA and 2.5 million DNA molecules per murine lung cell, if delivery to the lung is 100% efficient. Control mice were treated identically with either blank nanoparticles without nucleic acid cargo, or with nanoparticles containing PNA/DNA targeting human β-globin. While a scrambled PNA would provide the most closely matched molecular control, this off-target PNA provides a control of effects from non-specific PNA activity. Each independently performed experiment included at least one CF-targeted PNA/DNA treated mouse and one control mouse. All procedures were performed in compliance with relevant laws and institutional guidelines, and were approved by the Yale University Institutional Animal Care and Use Committee.

Nasal potential differences (NPDs) were measured as previously described (Egan, et al., Science, 304:600-602 (2004)). Briefly, mice were anesthetized with ketamine/xylazine, and one electrode probe placed into one nostril, with a reference electrode with 3% agar in Ringer's solution placed subcutaneously. A microperfusion pump was used to flow solution through the electrode probe at 0.2 mL/hour. Potential differences were measured first with a control Ringer's solution, then with Ringer's solution containing 100 μM amiloride, then a chloride-free solution with amiloride, and then chloride-free solution with amiloride and forskolin/IBMX. NPDs were measured prior to and after the nanoparticle treatment.

Bronchoalveolar Lavage (BAL) Fluid Analysis and Lung Histology

BAL fluid was collected by standard protocols as previously described54, and cytokines measured using a microsphere-based multiplex assay per manufacturer instructions (Luminex; Millipore, Billerica, Mass.). To collect the lungs for histopathology, a midline incision from sternum to diaphragm was performed and, to remove blood from the pulmonary circulation, PBS was perfused via the right ventricle using a 20 g needle. Lungs were inflated with 0.5% low melt agarose at constant pressure, then removed from the chest and placed in fixative. Paraffin embedded tissues were stained with hemotoxylin and eosin stain for imaging.

To account for slight sequence variation between the mouse and human CFTR genes, new donor DNAs and PNAs were designed to target the mouse gene and correct the mouse F508del mutation (FIG. 1E). Binding of the mouse-specific PNA to the target DNA was confirmed by gel shift assay. PLGA and PLGA/PBAE/MPG nanoparticles were formulated to contain the mouse-specific triplex-forming PNA and donor DNA, and CF mice (Zeiher, et al., The Journal of Clinical Investigation, 96:2051-2064 (1995)) were treated with the nanoparticle suspension by intranasal application on days 1, 3, 6, and 9. Four days after the last treatment (day 14), correction of the mouse CFTR mutation in the nasal epithelium was assayed by measuring the nasal potential difference, a non-invasive assay used to detect chloride transport in vivo. Normally, CF nasal epithelia (human and mice) exhibit a large lumen negative nasal potential that is amiloride sensitive as well as a lack of activation of cyclic AMP stimulated chloride efflux. This can be contrasted with a more modest amiloride sensitive response and the presence of robust cyclic AMP stimulated chloride efflux in non-affected tissue. The lack of activation of cyclic AMP stimulated chloride flux is due directly to CFTR dysfunction and serves as a surrogate of CFTR activity.

After intranasal delivery of mCFPNA2/donor DNA containing nanoparticles, the impaired response to cyclic AMP stimulation was partially corrected, with mice exhibiting nasal potential differences that hyperpolarized in response to forskolin, which is more characteristic of wild-type mice. The degree of hyperpolarization in mice treated with unmodified PLGA nanoparticles containing mCFPNA2/donor DNA was modest and did not reach statistical significance, while treatment with PLGA/PBAE/MPG nanoparticles demonstrated a significant change in NPD (p=0.004) (FIG. 4B). After intranasal delivery with PLGA/PBAE/MPG nanoparticles, the response to cyclic AMP stimulation was much more robust, with mice exhibiting a significant increase in their response to forskolin (FIG. 4C).

No significant change was seen in mice treated in parallel with blank nanoparticles, or in mice treated in parallel with PNA/DNA containing PLGA/PBAE/MPG nanoparticles targeting an unrelated genomic target but with similar base composition (FIG. 4C). In these control experiments, additional CF mice were treated identically to the experimental group with PLGA/PBAE/MPG nanoparticles containing either no nucleic acid cargo, or with PNA and DNA targeting human β-globin; these PNA had similar base composition as the CF-targeted DNA but with 12 mismatches out of 17 in the Watson-Crick domain. The j-globin PNA was shown to be functionally active for inducing gene editing in β-globin (McNeer, et al., Gene Ther., 20:658-659 (2013)) but had no effect on the CFTR gene. For comparison, cyclic AMP responses of the nasal potential difference assays in wild-type mice were more robust (FIG. 4C); this is expected given that wild-type mice have a homogenous population of wild-type CFTR-containing cells. In addition to the partial correction of the impaired cyclic AMP response a significant reduction in the large lumen negative nasal potential was observed in CF mice after treatment with PLGA/PBAE/MPG nanoparticles. This amiloride-sensitive portion of NPD was significantly reduced post treatment and similar in magnitude to that observed in wild-type mice (FIGS. 4E and 4F).

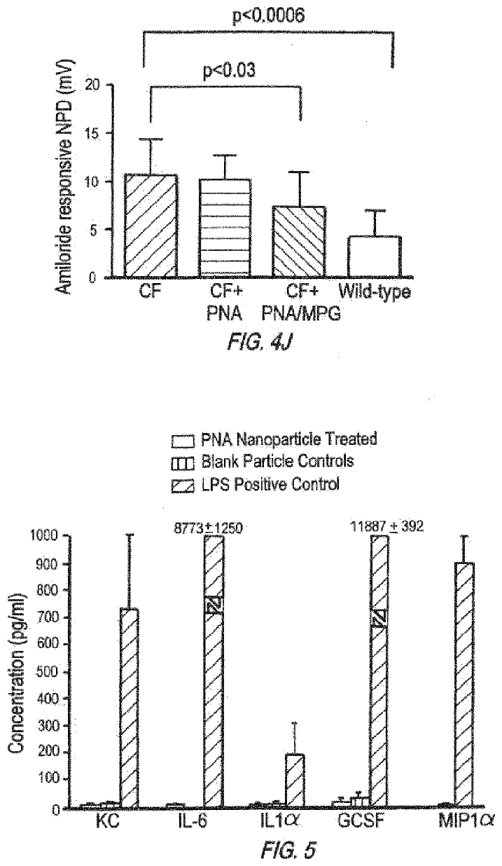

Finally, there was no increased production of inflammatory cytokines in bronchioalveolar lavage fluid of treated mice (FIG. 5), and lungs showed normal histology. Histology of limited nasal epithelial samples showed no obvious differences between treated and untreated mice. There was a reduction in inflammatory cells in the bronchioalveolar lavage (BAL) of CF mice treated with PLGA/PBAE/MPG nanoparticles when compared to untreated CF mice: for n=4 mice in each group, average BAL cell counts were 1.24×10⁵ in untreated CF mice, 0.4×10⁵ in treated CF mice, and 0.32×10⁵ for wildtype mice, p=0.03 for untreated versus treated CF mice.

### Example 5: Deep Sequencing Confirms Gene Modification

Modification was further confirmed in nanoparticle-treated human CFBE cells and in nanoparticle-treated mouse nasal epithelium and lung by deep sequencing, which allows for sequencing of millions of individual CFTR gene alleles in populations of cells (Table 2). In human CFBE cells treated in vitro serially three times with PLGA/PBAE/MPG particles, targeted modification frequency approached 10%. Increased efficiency of PLGA/PBAE/MPG nanoparticles over PLGA nanoparticles was also confirmed. In mice treated serially with PLGA/PBAE/MPG nanoparticles as described above, modification in the nasal epithelium was more than 5%, and more than 1% in the lung (Table 2); modification was not detected in vivo when plain PLGA nanoparticles were used. In addition, deep sequencing of cDNA amplicons produced from lung mRNA detected at least greater than 80-fold higher expression of corrected CFTR RNA in a treated mouse (PLGA/PBAE/MPG particles) versus untreated, demonstrating that the modification was present at the mRNA level, consistent with findings of functional correction.

TABLE 2
Deep sequencing confirms efficient modification with low off-target effects

| | | Modified CFTR | | Modified off- | % Off- |
|---|---|---|---|---|---|
| | Sample | sequences | % Correction | target sequences | Target |
| in vitro human | Control CFBE | 0/1894182 | <0.0005% | 0/1102030 | <0.00009% |
| CFBE cells | PLGA Nanoparticles | 1502/1016551 | 0.15% | 6/236874 | <0.0004% |
| | PBAE/PLGA/MPG | 947458/10279296 | 9.2% | 0/10304922 | <0.00001% |
| | Nanoparticles | | | | |
| in vivo CF mouse | Control nasal | 0/46633 | <0.002% | 0/517496 | <0.0002% |
| model | epithelium | | | | |
| | Control lung | 0/1385709 | <0.0001% | 0/125970 | <0.001% |
| | PLGA Nanoparticles - | 0/406270 | <0.00025% | | |
| | Nasal Epithelium | | | | |
| | PBAE/PLGA/MPG | 31092/547521 | 5.7% | 0/1380607 | <0.0001% |
| | Nanoparticles - | | | | |
| | nasal epithelium | | | | |
| | PBAE/PLGA/MPG | 9052/732024 | 1.2% | 0/1385709 | <0.0001% |
| | Nanoparticles - | | | | |
| | lung | | | | |

### Example 6: Off-Target Effects are Low

### Materials and Methods

Comet Assay

300000 CFBE cells/well were pated on 6-well plates in 1 mL media, then treated with 2 mg/mL of PBAE/MPG/PLGA nanoparticles either with DNA alone or both DNA and PNA, or with lipofectamine to deliver 2 ug of human cas9 plasmid #41815 (Addgene, Cambridge, Mass.) (Mali, et al., Science, 339:823-826 (2013)). After 24 hours, cells were scraped and harvested, and prepared using the Trevigen CometAssay kit per manufacturer protocol (Trevigen, Gaithersburg, Md.). Briefly, cells were suspended in agarose, added to comet slides, allowed to set, incubated 1 hr in lysis solution, placed in electrophoresis solution for 30 min, then run at 21 V for 45 min, placed in acetate solution for 30 min, 70% ethanol solution for 30 min, dried, stained with Sybr Green for 30 min, then visualized using an EVOS microscope. TriTek Comet Score freeware was used to analyze images.

### Results

In addition, off-target modification in sites partially homologous to CFTR was examined. A section of chromosome 4 with 80% homology to the human donor DNA was queried in human cells (flanking features included a type II inositol-3,4-bisphosphate 4-phosphatase and a ubiquitin carboxyl-terminal hydrolase), and a similar section of the X chromosome with 50% homology to the donor DNA sequence (uncharacterized proteins) was queried in mice. In millions of sequenced alleles at these sites, there were no detected mutations above the machine-specific error rate (Table 3). In addition, thirteen additional off-target sites in the human genome with partial homology (>14 bp) to hCFPNA2 were queried in treated CFBE cells by deep sequencing. In these thirteen additional sites, off-target mutation/error rates were similar to untreated controls (FIG. 6A). For instance, for both untreated and treated cells, approximately 80+/−15% (average across the 13 sites) of queried sequences had zero mismatches (no difference above the machine-specific error rate between samples), and similarly there were no differences in the number of sequences with one to five mismatches in the queried sites. No differences in mutation frequencies above the error rate were seen at the individual sites.

Finally, a single-cell gel electrophoresis assay (comet assay) was used to assess for the presence of DNA double-stranded breaks (FIG. 6B). In this assay, electrophoresis of lysed cells results in migration of fragmented DNA, producing images that resemble comets when observed by fluorescent microscopy, with the length of the comet “tail” corresponding to the number of DNA breaks. No difference was seen between cells treated with DNA-containing and PNA/DNA-containing nanoparticles. In contrast, there was a slight but statistically significant increase in comet tail moments in cells treated with a human codon-optimized Cas9 expression plasmid (Mali, et al., Science, 339:823-826 (2013)), which is designed to express CRISPR associated protein 9, the DNA nuclease used in CRISPR-based gene editing technologies that induces double stranded breaks.

The experiments above exemplify three PNA molecules designed to bind to the human CFTR gene at sites within 350 base pairs of the F508del mutation. These sites were chosen because homopurine/homopyrimidine sites are needed for Hoogsteen binding and triple helix formation, and previous studies indicate triplex-forming PNAs can increase levels of gene recombination at sites up to 750 base pairs (bp) from the target, with drop-off when the target is further than 400 bp away (Knauert, et al., Biochemistry, 44:3856-3864 (2005)). While all three PNA molecules were found to bind to their respective targeted sites in CFTR, hCFPNA2 induced the most consistent gene modification in CFTR in conjunction with the donor DNA as detected by AS-PCR. In prior work, some variability in the ability of certain triplex-forming PNA molecules to induce gene modification was noted (Chin, et al., Proc Natl Acad Sci USA, 105:13514-13519 (2008), Knauert, et al., Biochemistry, 44:3856-3864 (2005)). Factors which may contribute to differences in PNA intracellular activity include accessibility of the binding site in the cellular chromatin, folding dynamics of the molecules being used, and strength of binding in intracellular conditions.

Cloning by limiting dilution of nanoparticle-treated cells allowed interrogation of gene correction at the level of individual cells. Modification was passed on to cell progeny through months of cloning, demonstrating heritability. Gene modification in these positive clones was further confirmed by direct sequencing, and deep sequencing. CFTR gene correction in the positive clones was also confirmed by the presence of sequence-corrected mRNA and by functional analysis in an MQAE chloride flux assay. Positive clones had increased chloride flux in comparison to untreated F508del CFBE cells.

Modified PLGA/PBAE/MPG nanoparticles loaded with the PNA and donor DNA showed improved activity over PLGA nanoparticles, as demonstrated by MQAE chloride flux and deep sequencing both in vitro and in vivo. Of note, PLGA/PBAE/MPG nanoparticles carrying PNA/DNA cargo targeting an unrelated genomic site did not produce changes in chloride flux either in vitro or in vivo, indicating that the observed effects are due to gene modification rather than a non-specific physiologic effect. In other work, using nanoparticles loaded with fluorescent dyes as tracers, intranasal administration of PLGA/PBAE/MPG nanoparticles was shown to produced significantly greater nanoparticle association with airway epithelial cells than PLGA nanoparticles (Fields, et al., Advanced Healthcare Materials, 4:361-366 (2015)).

After multiple in vitro treatments with PLGA/PBAE/MPG nanoparticles, chloride efflux of CFBE cells approached that of normal human bronchial epithelial cells, and modification frequencies of up to 25% based on functional chloride efflux. However, it is possible that some individual cells with increased efflux may have had enhanced chloride transport due to a bystander effect from being adjacent to corrected cells, and not from direct modification. In addition, it is possible that corrected cells have a selection advantage, resulting in their preferential expansion. Deep sequencing showed modification up to 10% and no off-target effects above background mutation/read errors rates in untreated cells as assessed in 13 sites with partial homology for possible off-target binding of the PNA. In addition, no increased DNA damage was detected in treated cells by comet assay. Tail-clamp PNA molecules have very low levels of binding to mismatched sites and do not have any intrinsic nuclease activity. Unlike nuclease-based approaches to gene editing like zinc-finger nuclease and CRISPR, PNAs do not directly make strand breaks but instead provoke endogenous DNA repair pathways in the cell to mediate sequence conversion and gene correction that is templated by the co-introduced donor DNA. A low frequency of off-target effects will be of utmost importance for gene editing in this chronic, systemic disease.

In addition, surface-modified PLGA/PBAE/MPG nanoparticles showed greater genome engineering capacity after direct in vivo administration. Multiple intranasal treatments with PLGA/PBAE/MPG nanoparticles containing the murine CFTR-specific triplex-forming PNAs and donor DNAs were found to significantly modify the characteristic nasal potential difference defect in CF mice. Modification frequencies were greater than 5% in the nasal epithelium and 1% in the lung, with no detectable off-target mutations in a partially homologous site. There was no enhanced inflammatory cytokine production or changes in lung histology, highlighting the low immunogenicity of the approach. Because of this low toxicity, it is believed that longer courses of treatment are feasible and should enhance gene modification. Since correction of only one defective allele is required for restoration of chloride flux in cells, and studies have indicated that as little as 6-10% of cells need to be corrected for normal levels of ion transport in culture (Johnson, et al., Nature Genetics, 2:21-25 (1992)). According, the disclosed system has the potential to achieve gene correction at a clinically relevant level.

1. A triplex forming composition comprising one or more oligonucleotides that the bind or hybridize to a target region sequence in the human cystic fibrosis transmembrane conductance regulator (CFTR) gene of cell comprising TTTCCTCT (SEQ ID NO:70), TTTCCTCTATGGGTAAG (SEQ ID NO:71), AGAGGAAA (SEQ ID NO:72), CTTACCCATAGAGGAAA (SEQ ID NO:73), AGAAGAGG (SEQ ID NO:74), ATGCCAACTAGAAGAGG (SEQ ID NO:75), CCTCTTCT (SEQ ID NO:76), CCTCTTCTAGTTGGCAT (SEQ ID NO:77), CTTTCCCTT (SEQ ID NO:78), CTTTCCCTTGTATCTTTT (SEQ ID NO:79), AAGGGAAAG (SEQ ID NO:80), or AAAAGATAC AAGGGAAAG (SEQ ID NO:81).

2. The triplex forming composition of claim 1 comprising a triplex forming oligonucleotide substantially complementary to the target region sequence the can form a triple helix with double-stranded DNA at the target sequence based on the third strand binding code.

3. The triplex forming composition of claim 1 comprising a Hoogsteen binding peptide nucleic acid (PNA) segment and a Watson-Crick binding PNA segment collectively totaling no more than 50 nucleobases in length, wherein the two segments can bind or hybridize to the target region sequence comprising
(i)
(SEQ ID NO: 72)
5′-AGAGGAAA-3′,
(ii)
(SEQ ID NO: 73)
5′-CTTACCCATAGAGGAAA-3′
(iii)
(SEQ ID NO: 74)
5′-AGAAGAGG-3′,
(iv)
(SEQ ID NO: 75)
5′-ATGCCAACTAGAAGAGG-3′,
(v)
(SEQ ID NO: 80)
5′-AAGGGAAAG-3′,
or
(iv)
(SEQ ID NO: 81)
5′-AAAAGATACAAGGGAAAG-3′,
in a cell's genome to induce strand invasion, displacement, and formation of a triple-stranded molecule among the two PNA segments and the target region's sequence,
wherein the Hoogsteen binding segment binds to the target duplex by Hoogsteen binding for a length of least five nucleobases, and
wherein the Watson-Crick binding segment binds to the target duplex by Watson-Crick binding for a length of least five nucleobases.

4. The triplex forming composition of claim 3, wherein the Hoogsteen binding segment comprises one or more chemically modified cytosines selected from the group consisting of pseudocytosine, pseudoisocytosine, and 5-methylcytosine.

5. The triplex forming composition of claim 2, wherein the Watson-Crick binding segment comprises a tail sequence of up to fifteen nucleobases that binds to the target duplex by Watson-Crick binding outside of the triplex.

6. The triplex forming composition of claim 3 wherein the two segments are linked by a linker.

7. The triplex forming composition of claim 6, wherein the linker is between 1 and 10 units of 8-amino-3,6-dioxaoctanoic acid.

8. The triplex forming composition of claim 3, wherein the
(i) the Hoogsteen binding segment comprises the sequence TJTJJTTT (SEQ ID NO:91) and the Watson-Crick binding segment comprises the sequence TTTCCTCT (SEQ ID NO:83) or TTTCCTCTATGGGTAAG (SEQ ID NO:84);
(ii) the Hoogsteen binding segment comprises the sequence TJTTJTJJ (SEQ ID NO: 177) and the Watson-Crick binding segment comprises the sequence CCTCTTCT (SEQ ID NO:86), or CCTCTTCTAGTTGGCAT (SEQ ID NO:87); or
(iii) the Hoogsteen binding segment comprises the sequence TTJJJTTTJ (SEQ ID NO:92) and the Watson-Crick binding segment comprises the sequence CTTTCCCTT (SEQ ID NO:89), or CTTTCCCTTGTATCTTTT (SEQ ID NO:90);
wherein “J” is pseudoisocytosine.

9. The triplex forming composition of claim 6, wherein the segments are linked and form a molecule having the sequence
(i)(hCFPNA2)
(SEQ ID NO: 93)
lys-lys-lys-TJTJJTTT-OOO-TTTCCTCT
ATGGGTAAG-lys-lys-lys;
(ii)(hCFPNA1)
(SEQ ID NO: 94)
lys-lys-lys-TJTTJTJJ-OOO-CCTCTTCT
AGTTGGCAT-lys-lys-lys;
(iii)(hCFPNA3)
(SEQ ID NO: 95)
lys-lys-lys-TTJJJTTTJ-OOO-CTTTCCC
TTGTATCTTTT-lys-lys-lys,

10. The triplex forming composition of claim 1 further comprising a donor oligonucleotide comprising a sequence that can correct a mutation(s) in the CFTR gene by triplex forming molecule-induced or enhanced recombination.

11. The triplex forming composition of claim 10, wherein the donor comprises the sequence 5′TTCTGTATCTATATTCATCATAGGAAACACCAAAGATAATGTTCTCCTTAATGGTG CCAGG3′ (SEQ ID NO:96), or a functional fragment thereof that is suitable and sufficient to correct the F508del mutation in the CFTR gene.

12. The triplex forming composition of claim 10 further comprising nanoparticles, wherein the PNA segments, the donor oligonucleotide, or a combination thereof are packaged together or separately in nanoparticles.

13. The triplex forming composition of claim 12, wherein the nanoparticles comprise polyhydroxy acids.

14. The triplex forming composition of claim 13, wherein the nanoparticles comprise poly(lactic-co-glycolic acid) (PLGA).

15. The triplex forming composition of claim 14, wherein the nanoparticle comprise a blend of PLGA and poly(beta-amino) esters (PBAEs) comprising about between about 5 and about 25 percent PBAE (wt %).

16. The triplex forming composition of claim 12, wherein the nanoparticle is prepared by double emulsion.

17. The triplex forming composition of claim 12 further comprising a targeting moiety, a cell penetrating peptide, or a combination thereof associated with, linked, conjugated, or otherwise attached directly or indirectly to the PNA segments or the nanoparticles.

18. The triplex forming composition of claim 17, wherein the cell penetrating peptide comprises the sequence GALFLGFLGAAGSTMGAWS QPKKKRKV (SEQ ID NO: 12) (MPG (Synthetic chimera: SV40 Lg T. Ant.+HIV gb41 coat)).

19. A method of modifying the human cystic fibrosis transmembrane conductance regulator (CFTR) gene in a cell comprising administering a subject with a mutation in the CFTR gene an effective amount of the triplex forming composition according to claim 10 to increase correction of the mutation in a population of cells relative to contacting the cells with donor oligonucleotide alone.

20. The method of claim 19, wherein the triplex forming composition is administered by intranasal or pulmonary delivery.

21. The method of claim 20, wherein the composition induces or enhances gene correction in an effective amount to reduce one or more symptoms of cystic fibrosis.

22. The method of claim 21, wherein composition is administered in an effective amount to improve impaired response to cyclic AMP stimulation, improve hyperpolarization in response to forskolin, reduction in the large lumen negative nasal potential, reduction in inflammatory cells in the bronchioalveolar lavage (BAL), improve lung histology, or a combination thereof.


SEQUENCE LISTING (177 sequences)

SEQ ID NO: 1 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 2 - protein, 165 aa, Homo sapiens
MEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 3 - protein, 273 aa, Mus musculus
MKKTQTWIITCIYLQLLLFNPLVKTKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVAASSLRNDSSSSNRKAAKAPEDSGLQWTAMALPALISLVIGFAFGALYWKKKQSSLTRAVENIQINEEDNEISMLQQKEREFQEV

SEQ ID NO: 4 - protein, 165 aa, Mus musculus
MKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVA

SEQ ID NO: 5 - protein, 273 aa, Mus musculus
MKKTQTWIITCIYLQLLLFNPLVKTQEICRNPVTDNVKDITKLVANLPNDYMITLNYVAGMDVLPSHCWLRDMVTHLSVSLTTLLDKFSNISEGLSNYSIIDKLGKIVDDLVACMEENAPKNVKESLKKPETRNFTPEEFFSIFNRSIDAFKDFMVASDTSDCVLSSTLGPEKDSRVSVTKPFMLPPVAASSLRNDSSSSNRKAAKSPEDPGLQWTAMALPALISLVIGFAFGALYWKKKQSSLTRAVENIQINEEDNEISMLQQKEREFQEV

SEQ ID NO: 6 - protein, 165 aa, Rattus rattus
MQEICRNPVTDNVKDITKLVANLPNDYMITLNYVAGMDVLPSHCWLRDMVTHLSVSLTTLLDKFSNISEGLSNYSIIDKLGKIVDDLVACMEENAPKNVKESLKKPETRNFTPEEFFSIFNRSIDAFKDFMVASDTSDCVLSSTLGPEKDSRVSVTKPFMLPPVA

SEQ ID NO: 7 - protein, 11 aa, Artificial Sequence
  Synthetic peptide
YGRKKRRQRRR

SEQ ID NO: 8 - protein, 9 aa, Artificial Sequence
  Synthetic Peptide
RKKRRQRRR

SEQ ID NO: 9 - protein, 16 aa, Artificial Sequence
  Synthetic Peptide
RQIKIWFQNRRMKWKK

SEQ ID NO: 10 - protein, 19 aa, Artificial Sequence
  Synthetic Peptide
HHHHRKKRRQRRRRHHHHH

SEQ ID NO: 11 - protein, 30 aa, Artificial Sequence
  Synthetic Peptide
MVKSKIGSWILVLFVAMWSDVGLCKKRPKP

SEQ ID NO: 12 - protein, 27 aa, Artificial Sequence
  Synthetic Peptide
GALFLGFLGAAGSTMGAWSQPKKKRKV

SEQ ID NO: 13 - DNA, 6060 bp, Homo sapiens
aaagctcttgctttgacaattttggtctttcagaatactataaatataacctatattataatttcataaagtctgtgcattttctttgacccaggatatttgcaaaagacatattcaaacttccgcagaacactttatttcacatatacatgcctcttatatcagggatgtgaaacagggtcttgaaaactgtctaaatctaaaacaatgctaatgcaggtttaaatttaataaaataaaatccaaaatctaacagccaagtcaaatctgtatgttttaacatttaaaatattttaaagacgtcttttcccaggattcaacatgtgaaatcttttctcagggatacacgtgtgcctagatcctcattgctttagttttttacagaggaatgaatataaaaagaaaatacttaaattttatccctcttacctctataatcatacataggcataattttttaacctaggctccagatagccatagaagaaccaaacactttctgcgtgtgtgagaataatcagagtgagattttttcacaagtacctgatgagggttgagacaggtagaaaaagtgagagatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgactgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacatatatatatatattttttttcttttcttaccagaaggttttaatccaaataaggagaagatatgcttagaactgaggtagagttttcatccattctgtcctgtaagtattttgcatattctggagacgcaggaagagatccatctacatatcccaaagctgaattatggtagacaaagctcttccacttttagtgcatcaatttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaagggggctttttgtagcttgatattcactactgtcttattaccctatcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaacctaaaatgaaatgaaatcagacccttgtcttacaccataaacaaaaataaatttgaatgggttaaagaattaaactaagacctaaaaccataaaaatttttaaagaaatcaaaagaagaaaattctaatattcatgttgcagccgttttttgaatttgatatgagaagcaaaggcaacaaaaggaaaaataaagaagtgaggctacatcaaactaaaaaatttccacacaaaaaagaaaacaatgaacaaatgaaaggtgaaccatgaaatggcatatttgcaaaccaaatatttcttaaatattttggttaatatccaaaatatataagaaacacagatgattcaataacaaacaaaaaattaaaaataggaaaataaaaaaattaaaaagaagaaaatcctgccatttatgcgagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaattctagatatctaatgtacagcatcgtgactgtagttaattgtactgtaagtatatttaaaatttgcaaagagagtagatttttttgtttttttagatggagttttgctcttgttgtccaggctggagtgcaatggcaagatcttggctcactgcaacctccgcctcctgggttcaagcaaatctcctgcctcagcctcccgagtagctgggattacaggcatgcgacaccatgcccagctaattttgtatttttagtagagacggggtttctccatgttggtcaggctgatccgcctcctcggccaccaaagggctgggattacaggcgtgaccaccgggcctggccgagagtagatcttaaaagcatttaccacaagaaaaaggtaactatgtgagataatgggtatgttaattagcttgattgtggtaatcatttcacaaggtatacatatattaaaacatcatgttgtacaccttaaatatatacaatttttatttgtgaatgatacctcaataaagttgaagaataataaaaaagaatagacatcacatgaattaaaaaactaaaaaataaaaaaatgcatcttgatgattagaattgcattcttgatttttcagatacaaatatccatttgactg

SEQ ID NO: 14 - DNA, 850 bp, Homo sapiens
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 15 - DNA, 12 bp, Artificial Sequence
  Synthetic Primer
gaaagaaagaga

SEQ ID NO: 16 - DNA, 18 bp, Artificial Sequence
  Synthetic Primer
tgccctgaaagaaagaga

SEQ ID NO: 17 - DNA, 7 bp, Artificial Sequence
  Synthetic Primer
ggagaaa

SEQ ID NO: 18 - DNA, 17 bp, Artificial Sequence
  Synthetic Primer
agaatggtgcaaagagg

SEQ ID NO: 19 - DNA, 7 bp, Artificial Sequence
  Synthetic Primer
aaaaggg

SEQ ID NO: 20 - DNA, 18 bp, Artificial Sequence
  Synthetic Primer
acatgattagcaaaaggg

SEQ ID NO: 21 - DNA, 12 bp, Artificial Sequence
  Synthetic Primer
ctttctttctct

SEQ ID NO: 22 - DNA, 12 bp, Artificial Sequence
  Synthetic Primer
tctctttctttc

SEQ ID NO: 23 - DNA, 18 bp, Artificial Sequence
  Synthetic Primer
tctctttctttcagggca

SEQ ID NO: 24 - DNA, 6 bp, Artificial Sequence
  Synthetic Primer
tttccc

SEQ ID NO: 25 - DNA, 7 bp, Artificial Sequence
  Synthetic Primer
ccctttt

SEQ ID NO: 26 - DNA, 18 bp, Artificial Sequence
  Synthetic Primer
cccttttgctaatcatgt

SEQ ID NO: 27 - DNA, 7 bp, Artificial Sequence
  Synthetic Primer
tttctcc

SEQ ID NO: 28 - DNA, 7 bp, Artificial Sequence
  Synthetic Primer
cctcttt

SEQ ID NO: 29 - DNA, 17 bp, Artificial Sequence
  Synthetic Primer
cctctttgcaccattct

SEQ ID NO: 30 - DNA, 12 bp, Artificial Sequence
  Synthetic Primer
  misc_feature (1)..(1): n = Pseudoisocytosine
  misc_feature (5)..(5): n = Pseudoisocytosine
  misc_feature (9)..(9): n = Pseudoisocytosine
  misc_feature (11)..(11): n = Pseudoisocytosine
ntttntttntnt

SEQ ID NO: 31 - DNA, 7 bp, Artificial Sequence
  Synthetic Primer
  misc_feature (5)..(7): n = Pseudoisocytosine
ttttnnn

SEQ ID NO: 32 - DNA, 7 bp, Artificial Sequence
  Synthetic Primer
  misc_feature (4)..(4): n = Pseudoisocytosine
  misc_feature (6)..(7): n = Pseudoisocytosine
tttntnn

SEQ ID NO: 33 - DNA, 30 bp, Artificial Sequence
  Synthetic Peptide Nucleic Acid
  misc_feature (1)..(1): n = Pseudoisocytosine
  misc_feature (1)..(1): Linked to lys-lys-lys
  misc_feature (5)..(5): n = Pseudoisocytosine
  misc_feature (9)..(9): n = Pseudoisocytosine
  misc_feature (11)..(11): n = Pseudoisocytosine
  misc_feature (12)..(13): Linked by three 8-amino-2, 6, 10-trioxaoctanoic acid, three 8-amino-3,6-dioxaoctanoic acid, or three 6-aminohexanoic acid molecules
  misc_feature (30)..(30): Linked to lys-lys-lys
ntttntttntnttctctttctttcagggca

SEQ ID NO: 34 - DNA, 25 bp, Artificial Sequence
  Synthetic Peptide Nucleic Acid
  misc_feature (1)..(1): Linked to lys-lys-lys
  misc_feature (5)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ttttnnncccttttgctaatcatgt

SEQ ID NO: 35 - DNA, 24 bp, Artificial Sequence
  Synthetic Peptide Nucleic Acid
  misc_feature (1)..(1): Linked to lys-lys-lys
  misc_feature (4)..(4): n = Pseudoisocytosine
  misc_feature (6)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (24)..(24): Linked to lys-lys-lys
tttntnncctctttgcaccattct

SEQ ID NO: 36 - DNA, 10 bp, Artificial Sequence
  Synthetic Primer
  misc_feature (2)..(2): n = Pseudoisocytosine
  misc_feature (7)..(7): n = Pseudoisocytosine
  misc_feature (10)..(10): n = Pseudoisocytosine
tnttttnttn

SEQ ID NO: 37 - DNA, 10 bp, Artificial Sequence
  Synthetic Primer
cttcttttct

SEQ ID NO: 38 - DNA, 10 bp, Artificial Sequence
  Synthetic Primer
  misc_feature (3)..(3): n = Pseudoisocytosine
  misc_feature (6)..(6): n = Pseudoisocytosine
  misc_feature (10)..(10): n = Pseudoisocytosine
ttnttntttn

SEQ ID NO: 39 - DNA, 10 bp, Artificial Sequence
  Synthetic Primer
ctttcttctt

SEQ ID NO: 40 - DNA, 10 bp, Artificial Sequence
  Synthetic Primer
  misc_feature (1)..(3): n = Pseudoisocytosine
  misc_feature (5)..(6): n = Pseudoisocytosine
  misc_feature (9)..(9): n = Pseudoisocytosine
nnntnnttnt

SEQ ID NO: 41 - DNA, 10 bp, Artificial Sequence
  Synthetic Primer
tcttcctccc

SEQ ID NO: 42 - DNA, 20 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (7)..(7): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
tnttttnttncttcttttct

SEQ ID NO: 43 - DNA, 20 bp, Artificial Sequence
  Synthetic Peptide Nucleic Acid
  misc_feature (1)..(1): Linked to lys-lys-lys
  misc_feature (3)..(3): n = Pseudoisocytosine
  misc_feature (6)..(6): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
ttnttntttnctttcttctt

SEQ ID NO: 44 - DNA, 20 bp, Artificial Sequence
  Synthetic Peptide Nucleic Acid
  misc_feature (1)..(1): Linked to lys-lys-lys
  misc_feature (1)..(3): n = Pseudoisocytosine
  misc_feature (5)..(6): n = Pseudoisocytosine
  misc_feature (9)..(9): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
nnntnnttnttcttcctccc

SEQ ID NO: 45 - DNA, 7 bp, Artificial Sequence
  Synthetic Primer
cctcttc

SEQ ID NO: 46 - DNA, 7 bp, Artificial Sequence
  Synthetic Primer
cttctcc

SEQ ID NO: 47 - DNA, 15 bp, Artificial Sequence
  Synthetic Primer
cttctccaaaggagt

SEQ ID NO: 48 - DNA, 18 bp, Artificial Sequence
  Synthetic Primer
cttctccacaggagtcag

SEQ ID NO: 49 - DNA, 7 bp, Artificial Sequence
  Synthetic Primer
ttcctct

SEQ ID NO: 50 - DNA, 7 bp, Artificial Sequence
  Synthetic Primer
tctcctt

SEQ ID NO: 51 - DNA, 15 bp, Artificial Sequence
  Synthetic Primer
tctccttaaacctgt

SEQ ID NO: 52 - DNA, 8 bp, Artificial Sequence
  Synthetic Primer
tctcttct

SEQ ID NO: 53 - DNA, 8 bp, Artificial Sequence
  Synthetic Primer
tcttctct

SEQ ID NO: 54 - DNA, 16 bp, Artificial Sequence
  Synthetic Primer
tcttctctgtctccac

SEQ ID NO: 55 - DNA, 18 bp, Artificial Sequence
  Synthetic Primer
tcttctctgtctccacat

SEQ ID NO: 56 - DNA, 7 bp, Artificial Sequence
  Synthetic Primer
  misc_feature (1)..(2): n = Pseudoisocytosine
  misc_feature (4)..(4): n = Pseudoisocytosine
  misc_feature (7)..(7): n = Pseudoisocytosine
nntnttn

SEQ ID NO: 57 - DNA, 22 bp, Artificial Sequence
  Synthetic Peptide Nucleic Acid
  misc_feature (1)..(1): Linked to lys-lys-lys
  misc_feature (3)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
ttnntnttctccttaaacctgt

SEQ ID NO: 58 - DNA, 24 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (24)..(24): Linked to lys-lys-lys
tntnttnttcttctctgtctccac

SEQ ID NO: 59 - DNA, 25 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
nntnttncttctccacaggagtcag

SEQ ID NO: 60 - DNA, 26 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (26)..(26): Linked to lys-lys-lys
tntnttnttcttctctgtctccacat

SEQ ID NO: 61 - DNA, 51 bp, Artificial Sequence
  Synthetic Primer
gttcagcgtgtccggcgagggcgaggtgagtctatgggacccttgatgttt

SEQ ID NO: 62 - DNA, 51 bp, Artificial Sequence
  Synthetic Primer
aaacatcaagggtcccatagactcacctcgccctcgccggacacgctgaac

SEQ ID NO: 63 - DNA, 70 bp, Artificial Sequence
  Synthetic Primer
cttgccccacagggcagtaacggcagatttttcttccggcgttaaatgcaccatggtgtctgtttgaggt

SEQ ID NO: 64 - DNA, 51 bp, Artificial Sequence
  Synthetic Primer
acagacaccatggtgcacctgactcctgaggagaagtctgccgttactgcc

SEQ ID NO: 65 - DNA, 60 bp, Artificial Sequence
  Synthetic Primer
aaagaataacagtgataatttctgggttaaggcaatagcaatatctctgcatataaatat

SEQ ID NO: 66 - DNA, 29 bp, Artificial Sequence
  synthetic polynucleotide
cctggcaccattaaggagaacattatctt

SEQ ID NO: 67 - DNA, 19 bp, Artificial Sequence
  synthetic polynucleotide
cccttttcaaggtgagtag

SEQ ID NO: 68 - DNA, 19 bp, Artificial Sequence
  synthetic polynucleotide
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid moleculess
  misc_feature (24)..(4): Linked to lys-lys-lys
ctactcaccttgaaaaggg

SEQ ID NO: 69 - DNA, 27 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (6)..(6): n = Pseudoisocytosine
  misc_feature (7)..(7): n = Pseudoisocytosine
  misc_feature (8)..(8): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
nttttnnncccttttcaaggtgagtag

SEQ ID NO: 70 - DNA, 8 bp, Artificial Sequence
  Synthetic Primer
tttcctct

SEQ ID NO: 71 - DNA, 17 bp, Artificial Sequence
  Synthetic Primer
tttcctctatgggtaag

SEQ ID NO: 72 - DNA, 8 bp, Artificial Sequence
  Synthetic Primer
agaggaaa

SEQ ID NO: 73 - DNA, 17 bp, Artificial Sequence
  Synthetic Primer
cttacccatagaggaaa

SEQ ID NO: 74 - DNA, 8 bp, Artificial Sequence
  Synthetic Primer
agaagagg

SEQ ID NO: 75 - DNA, 17 bp, Artificial Sequence
  Synthetic Primer
atgccaactagaagagg

SEQ ID NO: 76 - DNA, 8 bp, Artificial Sequence
  Synthetic Primer
cctcttct

SEQ ID NO: 77 - DNA, 17 bp, Artificial Sequence
  Synthetic Primer
cctcttctagttggcat

SEQ ID NO: 78 - DNA, 9 bp, Artificial Sequence
  Synthetic Primer
ctttccctt

SEQ ID NO: 79 - DNA, 18 bp, Artificial Sequence
  Synthetic Primer
ctttcccttgtatctttt

SEQ ID NO: 80 - DNA, 9 bp, Artificial Sequence
  Synthetic Primer
aagggaaag

SEQ ID NO: 81 - DNA, 18 bp, Artificial Sequence
  Synthetic Primer
aaaagatacaagggaaag

SEQ ID NO: 82 - DNA, 8 bp, Artificial Sequence
  Synthetic Primer
tctccttt

SEQ ID NO: 83 - DNA, 8 bp, Artificial Sequence
  Synthetic Primer
tttcctct

SEQ ID NO: 84 - DNA, 17 bp, Artificial Sequence
  Synthetic Primer
tttcctctatgggtaag

SEQ ID NO: 85 - DNA, 8 bp, Artificial Sequence
  Synthetic Primer
tcttctcc

SEQ ID NO: 86 - DNA, 8 bp, Artificial Sequence
  Synthetic Primer
cctcttct

SEQ ID NO: 87 - DNA, 17 bp, Artificial Sequence
  Synthetic Primer
cctcttctagttggcat

SEQ ID NO: 88 - DNA, 9 bp, Artificial Sequence
  Synthetic Primer
ttccctttc

SEQ ID NO: 89 - DNA, 9 bp, Artificial Sequence
  Synthetic Primer
ctttccctt

SEQ ID NO: 90 - DNA, 18 bp, Artificial Sequence
  Synthetic Primer
ctttcccttgtatctttt

SEQ ID NO: 91 - DNA, 8 bp, Artificial Sequence
  Synthetic Primer
  misc_feature (2)..(2): n = Pseudoisocytosine
  misc_feature (4)..(5): n = Pseudoisocytosine
tntnnttt

SEQ ID NO: 92 - DNA, 9 bp, Artificial Sequence
  Synthetic Primer
  misc_feature (3)..(5): n = Pseudoisocytosine
  misc_feature (9)..(9): n = Pseudoisocytosine
ttnnntttn

SEQ ID NO: 93 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(5): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tntnnttttttcctctatgggtaag

SEQ ID NO: 94 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (5)..(5): n = Pseudoisocytosine
  misc_feature (7)..(8): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tnttntnncctcttctagttggcat

SEQ ID NO: 95 - DNA, 27 bp, Artificial Sequence
  Synthetic Peptide Nucleic Acid
  misc_feature (1)..(1): Linked to lys-lys-lys
  misc_feature (3)..(5): n = Pseudoisocytosine
  misc_feature (9)..(9): n = Pseudoisocytosine
  misc_feature (9)..(10): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
ttnnntttnctttcccttgtatctttt

SEQ ID NO: 96 - DNA, 61 bp, Artificial Sequence
  Synthetic Primer
ttctgtatctatattcatcataggaaacaccaaagataatgttctccttaatggtgccagg

SEQ ID NO: 97 - DNA, 10 bp, Artificial Sequence
  Synthetic Primer
cttcctcttt

SEQ ID NO: 98 - DNA, 10 bp, Artificial Sequence
  Synthetic Primer
tttctccttc

SEQ ID NO: 99 - DNA, 18 bp, Artificial Sequence
  Synthetic Primer
tttctccttcagtgttca

SEQ ID NO: 100 - DNA, 7 bp, Artificial Sequence
  Synthetic Primer
ttttcct

SEQ ID NO: 101 - DNA, 7 bp, Artificial Sequence
  Synthetic Primer
tcctttt

SEQ ID NO: 102 - DNA, 20 bp, Artificial Sequence
  Synthetic Primer
tccttttgctcacctgtggt

SEQ ID NO: 103 - DNA, 10 bp, Artificial Sequence
  Synthetic Primer
tcttttttcc

SEQ ID NO: 104 - DNA, 10 bp, Artificial Sequence
  Synthetic Primer
ccttttttct

SEQ ID NO: 105 - DNA, 18 bp, Artificial Sequence
  Synthetic Primer
ccttttttctggctaagt

SEQ ID NO: 106 - DNA, 10 bp, Artificial Sequence
  Synthetic Primer
  misc_feature (1)..(1): n = Pseudoisocytosine
  misc_feature (4)..(5): n = Pseudoisocytosine
  misc_feature (7)..(7): n = Pseudoisocytosine
nttnntnttt

SEQ ID NO: 107 - DNA, 7 bp, Artificial Sequence
  Synthetic Primer
  misc_feature (5)..(6): n = Pseudoisocytosine
ttttnnt

SEQ ID NO: 108 - DNA, 10 bp, Artificial Sequence
  Synthetic Primer
  misc_feature (2)..(2): n = Pseudoisocytosine
  misc_feature (9)..(10): n = Pseudoisocytosine
tnttttttnn

SEQ ID NO: 109 - DNA, 58 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (55)..(56): Optional phosphorothioate internucleoside linkage
  misc_feature (56)..(57): Optional phosphorothioate internucleoside linkage
  misc_feature (57)..(58): Optional phosphorothioate internucleoside linkage
tgggattcaataaccttgcagacagtggaggaaggcctttggcgtgataccacaggtg

SEQ ID NO: 110 - DNA, 7 bp, Artificial Sequence
  Synthetic Primer
tcttttt

SEQ ID NO: 111 - DNA, 7 bp, Artificial Sequence
  Synthetic Primer
tttttct

SEQ ID NO: 112 - DNA, 18 bp, Artificial Sequence
  Synthetic Primer
tttttctgtaatttttaa

SEQ ID NO: 113 - DNA, 9 bp, Artificial Sequence
  Synthetic Primer
tctctttct

SEQ ID NO: 114 - DNA, 9 bp, Artificial Sequence
  Synthetic Primer
tctttctct

SEQ ID NO: 115 - DNA, 17 bp, Artificial Sequence
  Synthetic Primer
tctttctctgcaaactt

SEQ ID NO: 116 - DNA, 7 bp, Artificial Sequence
  Synthetic Primer
tttcttt

SEQ ID NO: 117 - DNA, 18 bp, Artificial Sequence
  Synthetic Primer
tttctttaagaacgagca

SEQ ID NO: 118 - DNA, 7 bp, Artificial Sequence
  Synthetic Primer
  misc_feature (2)..(2): n = Pseudoisocytosine
tnttttt

SEQ ID NO: 119 - DNA, 9 bp, Artificial Sequence
  Synthetic Primer
  misc_feature (2)..(2): n = Pseudoisocytosine
  misc_feature (4)..(4): n = Pseudoisocytosine
  misc_feature (8)..(8): n = Pseudoisocytosine
tntntttnt

SEQ ID NO: 120 - DNA, 7 bp, Artificial Sequence
  Synthetic Primer
  misc_feature (4)..(4): n = Pseudoisocytosine
tttnttt

SEQ ID NO: 121 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tnttttttttttctgtaatttttaa

SEQ ID NO: 122 - DNA, 26 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (8)..(8): n = Pseudoisocytosine
  misc_feature (9)..(10): Linked by three 8-amino-2, 6, 10-trioxaoctanoic acid, three 8-amino-3,6-dioxaoctanoic acid, or three 6-aminohexanoic acid molecules
  misc_feature (26)..(26): Linked to lys-lys-lys
tntntttnttctttctctgcaaactt

SEQ ID NO: 123 - DNA, 25 bp, Artificial Sequence
  Synthetic Peptide Nucleic Acid
  misc_feature (1)..(1): Linked to lys-lys-lys
  misc_feature (4)..(4): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tttnttttttctttaagaacgagca

SEQ ID NO: 124 - DNA, 60 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (57)..(58): Optional phosphorothioate internucleoside linkage
  misc_feature (58)..(59): Optional phosphorothioate internucleoside linkage
  misc_feature (59)..(60): Optional phosphorothioate internucleoside linkage
aagtttgcagagaaagataatatagtccttggagaaggaggaatcaccctgagtggaggt

SEQ ID NO: 125 - DNA, 10 bp, Artificial Sequence
  Synthetic Primer
ctcttcttct

SEQ ID NO: 126 - DNA, 10 bp, Artificial Sequence
  Synthetic Primer
tcttcttctc

SEQ ID NO: 127 - DNA, 15 bp, Artificial Sequence
  Synthetic Primer
tcttcttctcatttc

SEQ ID NO: 128 - DNA, 5 bp, Artificial Sequence
  Synthetic Primer
cttct

SEQ ID NO: 129 - DNA, 5 bp, Artificial Sequence
  Synthetic Primer
tcttc

SEQ ID NO: 130 - DNA, 10 bp, Artificial Sequence
  Synthetic Primer
tcttcttctc

SEQ ID NO: 131 - DNA, 15 bp, Artificial Sequence
  Synthetic Primer
tcttcttctcatttc

SEQ ID NO: 132 - DNA, 10 bp, Artificial Sequence
  Synthetic Primer
  misc_feature (1)..(1): n = Pseudoisocytosine
  misc_feature (3)..(3): n = Pseudoisocytosine
  misc_feature (6)..(6): n = Pseudoisocytosine
  misc_feature (9)..(9): n = Pseudoisocytosine
ntnttnttnt

SEQ ID NO: 133 - DNA, 5 bp, Artificial Sequence
  Synthetic Primer
  misc_feature (1)..(1): n = Pseudoisocytosine
  misc_feature (4)..(4): n = Pseudoisocytosine
nttnt

SEQ ID NO: 134 - DNA, 25 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (3)..(3): n = Pseudoisocytosine
  misc_feature (6)..(6): n = Pseudoisocytosine
  misc_feature (9)..(9): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ntnttnttnttcttcttctcatttc

SEQ ID NO: 135 - DNA, 20 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (4)..(4): n = Pseudoisocytosine
  misc_feature (5)..(6): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
nttnttcttcttctcatttc

SEQ ID NO: 136 - DNA, 60 bp, Artificial Sequence
  Synthetic Primer
attcccgagtagcagatgaccatgacagcttagggcaggaccagccccaagatgactatc

SEQ ID NO: 137 - DNA, 60 bp, Artificial Sequence
  Synthetic Primer
tttaggattcccgagtagcagatgacccctcagagcagcggcaggaccagccccaagatg

SEQ ID NO: 138 - DNA, 65 bp, Artificial Sequence
  Synthetic Primer
gatgactatctttaatgtctggaaattcttccagaattaattaagactgtatggaaaatgagagc

SEQ ID NO: 139 - DNA, 66 bp, Artificial Sequence
  Synthetic Primer
ccccaagatgactatctttaatgtctggaacgatcatcagaattgatactgactgtatggaaaatg

SEQ ID NO: 140 - DNA, 65 bp, Artificial Sequence
  Synthetic Primer
gatgactatctttaatgtctggaaattctactagaattgatactgactgtatggaaaatgagagc

SEQ ID NO: 141 - DNA, 12 bp, Artificial Sequence
  Synthetic Primer
ctgctcggaaga

SEQ ID NO: 142 - DNA, 12 bp, Artificial Sequence
  Synthetic Primer
tcttccgagcag

SEQ ID NO: 143 - DNA, 15 bp, Artificial Sequence
  Synthetic Primer
ccttcaccaagggga

SEQ ID NO: 144 - DNA, 15 bp, Artificial Sequence
  Synthetic Primer
tccccttggtgaagg

SEQ ID NO: 145 - DNA, 7 bp, Artificial Sequence
  Synthetic Primer
ttcccct

SEQ ID NO: 146 - DNA, 7 bp, Artificial Sequence
  Synthetic Primer
tcccctt

SEQ ID NO: 147 - DNA, 15 bp, Artificial Sequence
  Synthetic Primer
tccccttggtgaagg

SEQ ID NO: 148 - DNA, 7 bp, Artificial Sequence
  Synthetic Primer
  misc_feature (3)..(6): n = Pseudoisocytosine
ttnnnnt

SEQ ID NO: 149 - DNA, 63 bp, Artificial Sequence
  Synthetic PRimer
aggacggtcccggcctgcgacacttccgcccataattgttcttcatctgcggggcgggggggg

SEQ ID NO: 150 - DNA, 6 bp, Artificial Sequence
  Synthetic Primer
ccttct

SEQ ID NO: 151 - DNA, 6 bp, Artificial Sequence
  Synthetic Primer
tcttcc

SEQ ID NO: 152 - DNA, 12 bp, Artificial Sequence
  Synthetic Primer
tcttccgagcag

SEQ ID NO: 153 - DNA, 18 bp, Artificial Sequence
  Synthetic Primer
  misc_feature (1)..(1): Linked to lys-lys-lys
  misc_feature (1)..(2): n = Pseudoisocytosine
  misc_feature (5)..(5): n = Pseudoisocytosine
  misc_feature (6)..(7): Linked by three 8-amino-2, 6, 10-trioxaoctanoic acid, three 8-amino-3,6-dioxaoctanoic acid, or three 6-aminohexanoic acid molecules
  misc_feature (18)..(18): Linked to lys-lys-lys
nnttnttcttccgagcag

SEQ ID NO: 154 - DNA, 67 bp, Artificial Sequence
  Synthetic Primer
gggacggcgcccacataggccaaattcaattgctgatcccagcttaagacgtactggtcagcctggc

SEQ ID NO: 155 - DNA, 28 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (4)..(5): n = Pseudoisocytosine
  misc_feature (7)..(7): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (29)..(29): Linked to lys-lys-lys
nttnntnttttttctccttcagtgttca

SEQ ID NO: 156 - DNA, 27 bp, Artificial Sequence
  Synthetic Peptide Nucleic Acid
  misc_feature (1)..(1): Linked to lys-lys-lys
  misc_feature (5)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
ttttnnttccttttgctcacctgtggt

SEQ ID NO: 157 - DNA, 28 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (9)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (28)..(28): Linked to lys-lys-lys
tnttttttnnccttttttctggctaagt

SEQ ID NO: 158 - DNA, 22 bp, Artificial Sequence
  synthetic polynucleotide
cttctccacaggagtcaggtgc

SEQ ID NO: 159 - DNA, 22 bp, Artificial Sequence
  Synthetic Peptide Nucleic Acid
  misc_feature (1)..(1): Linked to lys-lys-lys
  misc_feature (3)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
ttnnnnttccccttggtgaagg

SEQ ID NO: 160 - DNA, 22 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
nntnttncttctccaaaggagt

SEQ ID NO: 161 - DNA, 29 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (29)..(29): Linked to lys-lys-lys
nntnttncttctccacaggagtcaggtgc

SEQ ID NO: 162 - DNA, 6 bp, Artificial Sequence
  synthetic polynucleotide
  misc_feature (1)..(2): n = pseudoisocytosine
  misc_feature (5)..(5): n = pseudoisocytosine
nnttnt

SEQ ID NO: 163 - DNA, 10 bp, Artificial Sequence
  Synthetic Primer
gaaggagaaa

SEQ ID NO: 164 - DNA, 7 bp, Artificial Sequence
  Synthetic Primer
aaaagga

SEQ ID NO: 165 - DNA, 10 bp, Artificial Sequence
  Synthetic Primer
agaaaaaagg

SEQ ID NO: 166 - DNA, 7 bp, Artificial Sequence
  Synthetic Primer
agaaaaa

SEQ ID NO: 167 - DNA, 9 bp, Artificial Sequence
  Synthetic Primer
agagaaaga

SEQ ID NO: 168 - DNA, 7 bp, Artificial Sequence
  Synthetic Primer
aaagaaa

SEQ ID NO: 169 - DNA, 61 bp, Artificial Sequence
  Synthetic Peptide Nucleic Acid
tcttatatctgtactcatcataggaaacaccaaagataatgttctccttgatagtacccgg

SEQ ID NO: 170 - DNA, 30 bp, Artificial Sequence
  synthetic polynucleotide
cctagttttgttagccatcagtttacagac

SEQ ID NO: 171 - DNA, 30 bp, Artificial Sequence
  synthetic polynucleotide
gcctggcaccattaaagaaaatatcattgg

SEQ ID NO: 172 - DNA, 18 bp, Artificial Sequence
  synthetic polynucleotide
tctccttaaacctgtctt

SEQ ID NO: 173 - DNA, 65 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (62)..(63): Optional phosphorothioate internucleoside linkage
  misc_feature (63)..(64): Optional phosphorothioate internucleoside linkage
  misc_feature (64)..(65): Optional phosphorothioate internucleoside linkage
ttgccccacagggcagtaacggcagacttctcctcaggagtcaggtgcaccatggtgtctgtttg

SEQ ID NO: 174 - DNA, 25 bp, Artificial Sequence
  Synthetic Peptide Nucleic Acid
  misc_feature (1)..(1): Linked to lys-lys-lys
  misc_feature (3)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ttnntnttctccttaaacctgtctt

SEQ ID NO: 175 - DNA, 7 bp, Artificial Sequence
  synthetic polynucleotide
  misc_feature (3)..(4): n = Pseudoisocytosine
  misc_feature (6)..(6): n = Pseudoisocytosine
ttnntnt

SEQ ID NO: 176 - DNA, 8 bp, Artificial Sequence
  synthetic polynucleotide
  misc_feature (2)..(2): n = Pseudoisocytosine
  misc_feature (4)..(4): n = Pseudoisocytosine
  misc_feature (7)..(7): n = Pseudoisocytosine
tntnttnt

SEQ ID NO: 177 - DNA, 8 bp, Artificial Sequence
  synthetic polynucleotide
  misc_feature (2)..(2): n = Pseudoisocytosine
  misc_feature (5)..(5): n = Pseudoisocytosine
  misc_feature (7)..(8): n = Pseudoisocytosine
tnttntnn